(19) United States
(12) Patent Application Publication
(10) Pub. No.: US 2025/0282728 A1
(43) Pub. Date: Sep. 11, 2025

(54) NITROGEN COMPRISING HETEROCYCLIC DERIVATIVES FOR THE TREATMENT OF DISORDERS ASSOCIATED WITH GPR55 RECEPTOR

(71) Applicant: Cerevance, Inc., Boston, MA (US)

(72) Inventors: Roland Burli, Cambridge (GB); Kevin Doyle, Cambridge (GB); Richard Hewer, Cambridge (GB); Martin Teall, Cambridge (GB)

(21) Appl. No.: 18/860,027

(22) PCT Filed: Apr. 26, 2023
(86) PCT No.: PCT/GB2023/051103

§ 371 Date: Oct. 25, 2024

(30) Foreign Application Priority Data
Apr. 26, 2022 (GB) 2206054.5

(51) Int. Cl. C07D 213/74; A61K 31/4439; A61K 31/444; A61K 31/497; A61K 31/501; A61K 31/506; A61K 31/53; C07D 401/04; C07D 403/04; C07D 413/04; C07D 471/04; C07D 498/04

(52) U.S. Cl. CPC C07D 213/74; A61K 31/4439; A61K 31/444; A61K 31/497; A61K 31/501; A61K 31/506; A61K 31/53; C07D 401/04; C07D 403/04; C07D 413/04; C07D 471/04; C07D 498/04

(57) ABSTRACT

The present invention provides compounds of formula (I) and pharmaceutically acceptable salts, solvates and prodrugs thereof: Formula (I) wherein n, R¹, R², R³, R⁴, R⁵, A¹, A², A³, A⁴ and A⁵ are as defined in the specification, processes for their preparation, pharmaceutical compositions containing them and their use in therapy, particularly for use in treating disorders associated with GPR₅₅ activity.

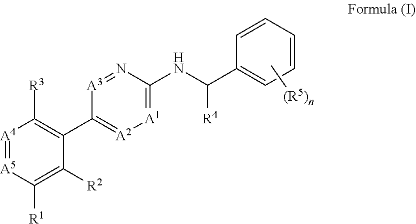

### FIELD OF THE INVENTION

The present invention relates to certain amines, processes for their preparation, pharmaceutical compositions containing them and their use in therapy, particularly for use in treating disorders associated with GPR55 activity.

### BACKGROUND OF THE INVENTION

GPR55 is a class A GPCR that was originally postulated to be a third cannabinoid receptor (Baker 2006). While GPR55 is shown to be modulated by cannabinoids, it is now considered that its endogenous ligand is lysophosphatidylinositol (LPI) with a particular sensitivity to the 2-arachidonoyl-LPI species (Ryberg 2007, Brown 2007, Oka 2007, Oka 2009, Henstridge 2009, Southern 2013). Additional endogenous ligands are reported by some publications including anandamide, 2-arachidonoylglycerol (2-AG), N-palmitoylethanolamine (PEA) (Ryberg 2007), and cannabidiol (CBD), which has been demonstrated to be a naturally-occurring antagonist of GPR55 (Ryberg 2007, Kaplan 2017). Receptor activation of GPR55 couples to the Gα_{12/13} pathway resulting in downstream activation of Rho GTPases, phospholipase C and calcium release from the endoplasmic reticulum (Ryberg 2007, Lauckner 2008, Henstridge 2009, Brown 2011).

In the brain, GPR55 expression is found in several regions including midbrain, basal ganglia and striatum, cortex, hippocampus, and hypothalamus, with highest expression in the striatum (Protein Atlas, GTEx, Ryberg 2007, Regard 2008, Henstridge 2011, Sylantyev 2013, Martinez-Pinilla 2014, Deliu 2015, Celorrio 2017, Marichal-Cancino 2017). Due to the clear CNS expression, role in response to endocannabinoids, and relevant publications as outlined below, GPR55 modulators are expected to be therapeutically beneficial in numerous neurological indications.

### Disease Indications for GPR55 Modulators

### Parkinson's Disease (PD)

The GPR55 receptor is expressed in the central nervous system, particularly in the striatum suggesting a potential role in motor function (Celorrio 2017). Indeed, GPR55 knockout mice have demonstrated impaired movement abilities when challenged with tasks requiring motor responses (Wu 2013). Of note, ablation of GPR55 function has not affected muscle strength, gross motor skills, motor learning, anxiety, or depressive behaviours. Celorrio and co-workers have further shown that abnormal cannabidiol (Abn-CBD, a known GPR55 agonist) prevented motor impairment in a mouse model of Parkinson's Disease, whereby animals were challenged with 1-methyl-4-phenyl-1,2,3,6-tetrahydropyridine and probenecid (MPTPp). This result was further confirmed in haloperidol-induced catalepsy, an alternative Parkinson's Disease mouse model: here, Abn-CBD as well as two further known GPR55 agonists (CID1792197, CID2440433) displayed significant anti-cataleptic effects (Celorrio 2017). Fatemi and co-workers (Fatemi 2021) independently reported the beneficial effects of GPR55 modulation in Parkinson's Disease: they tested the role of GPR55 in an experimental model of Parkinson's Disease, in which rats were challenged with 6-hydroxydopamine (6-OHDA). They demonstrated that injection of L-α-lysophosphatidylinositol (LPI, a known endogenous GPR55 agonist) into the striatum of the 6-OHDA-lesioned rats resulted in significantly improved locomotor performance, suggesting that striatal GPR55 function is involved in the motor control. More recently, these findings were confirmed using an alternative GPR55 activator molecule, VCE-006.1 (Burgaz 2021); here VCE-006.1 given chronically reversed the motor deficits induced by 6-OHDA and attenuated both the losses in tyrosine hydroxylase-containing neurons and the elevated glial reactivity seen in the substantia nigra. Taken together, these data demonstrate that activation of GPR55 is likely to be of therapeutic utility in Parkinson's Disease.

### Epilepsy

Several animal models demonstrate the ability of cannabinoids to prevent seizures and reduce mortality in epilepsy (Klein 2017). In humans, ameliorations in seizure activity are also observed with CBD treatment (Epidiolex, Greenwich Biosciences, a non-selective GPR55 antagonist) in Dravet and Lennox Gastaut syndromes (Devinsky 2017 and 2018, Thiele 2018). Endocannabinoid release has been shown to be upregulated in rodent models of epilepsy (Marsicano 2003, Wallace 2003) and a reduced occurrence of seizure discharges has been demonstrated in the hippocampus of rats treated with CBD (Izquierdo 1973). CBD can also restore activity of hippocampal neurons and prevent neuronal cell death in temporal lobe epilepsy (TLE) (Khan 2018, Do Val-da-Silva 2017). GPR55 has been shown to boost neurotransmitter release: at hippocampal CA3-CA1 synapses, activation of GPR55 by its endogenous ligand LPI, resulted in transient increases in efflux probability as a result of increased Ca²⁺ release from presynaptic stores (Sylantyev 2013). This effect was abolished by genetic deletion of GPR55 or with application of CBD. A second study by Rosenberg (2018), also found a postsynaptic effect of LPI via GPR55 receptors and hypothesised that LPI alters the excitatory/inhibitory ratio in hippocampal neuronal networks by a dual mechanism—enhancing excitation and attenuating inhibition.

An important finding in the coupling of the role of GPR55 in modulation of neuronal excitability to its role in pathophysiology of epilepsy, was the observation that expression of GPR55 was significantly increased in the hippocampus in a mouse model of epilepsy (Aso 2020). CBD enriched extract was able to decrease cognitive impairment in these mice but did not alter the severity of seizures. Concomitantly, in a mouse model of Dravet syndrome, CBD treatment increased inhibitory neurotransmission by blocking GPR55, resulting in an attenuation of epileptic seizures and social deficits (Kaplan 2017).

Although the mechanism of action appears to be complex and not fully elucidated, current literature does implicate GPR55 modulation as a potential therapeutic target for epilepsy.

### Anxiety

It is well established that the endocannabinoid system plays a significant role in regulating anxiety and stress related behaviours (Ruehle 2012, Yin 2019). As a central cannabinoid receptor that is expressed in brain regions implicated in anxiogenesis (e.g., striatum, hypothalamus, midbrain), GPR55 is well-positioned to modulate anxiety related disorders (Rahima 2015, Sharma 2013, Noronha 2017, Vizquez-León 2021). As such, its role in these disorders has recently been investigated by several independent groups, using the well characterised rodent anxiety tests, the elevated plus maze (EPM) and open field tests (OFT), in both naïve animals and those exposed to anxiety inducing treatments. In rats, treatment with the GPR55 agonist O-1602 increased time spent in, and entries made into the open arms of the EPM, reflecting an anxiolytic effect; whilst in contrast the GPR55 antagonist ML-193 produced opposing anxiogenic behaviours (Rahimi 2015). In this work, the O-1602 effects were shown to be fully inhibited by co-administration with ML-193. As both compounds are reported to show selectivity for GPR55 over other cannabinoid receptors (Ryberg 2007, Heynen-Genel 2010), these data provide strong evidence for a role of GPR55 in modulating anxiety. The role of GPR55 has been further explored in several rodent anxiety models. In a chronic restraint model in mice, GPR55 expression was shown to be significantly reduced in the cortex (Shi 2017). When subjected to short-term stress from acute restraint or forced swimming, the GPR55 agonist O-1602 again produced anxiolytic effects in EPM and OFT tests. In agreement with data from Rahimi (2015), these effects were blocked by a selective GPR55 antagonist (CID16020046; Kargl 2013). The specificity of O-1602 responses for GPR55 was further confirmed by Shi using shRNA gene silencing techniques (Shi 2017). In a mouse model of diet-induced obesity, obese mice demonstrated characteristic anxious behaviours, such as reduced time spent and distance travelled in open arms of the EPM. Treatment with a GPR55 agonist (LH21) was anxiolytic, relieving these obesity-associated phenotypes. To confirm the effects of LH21 were mediated by GPR55, healthy mice were co-dosed with LH21 and the selective antagonist CID16020046. The efficacy seen in EPM and OFT tests was fully inhibited by CID16020046 confirming the GPR55-mediated efficacy of LH21 (Romero-Zerbo 2017). Most recently, the role of GPR55 in anxiety was investigated in a rat defensive burying test in which animals were exposed to a GPR55 agonist (LPI) or antagonist (ML-193), injected directly into the periaqueductal grey of the midbrain (Vázquez-Le6n 2021). LPI injection produced an anxiolytic effect (e.g., decreased duration of burying, suppressed time of immobility after shock), whilst ML-193 was shown to produce anxiogenic behaviours (decreasing latency to burying, increasing duration of immobility after shock). Together, through pharmacological and genetic modulation of GPR55, these studies provide clear evidence for a role of GPR55 activation in decreasing anxiety related behaviours.

### Pain/Analgesia

GPR55 expression in subsets of dorsal root ganglion (DRG) and in brain periaqueductal gray (PAG) neurons has led to the suggestion that modulation of the receptor may be of therapeutic benefit in nociceptive function and pain. Activation results in increased intracellular calcium and subsequent downstream signalling events in both neuronal subtypes (Lauckner 2008). GPR55 knock out mice have no overt phenotype compared to wild type but do show differences in their response to various nociceptive stimuli in models of pain (Staton 2008, Bjursell 2016). Specifically, in the Freund's complete adjuvant (FCA) inflammatory model and a nerve ligation/neuropathic pain model, mechanical hyperalgesia was completely absent in knock out mice (Staton 2008) whilst displaying significantly faster reaction time in the tail flick test, indicative of thermal hyperalgesia (Bjursell 2016). Application of the endogenous ligand LPI depolarizes PAG neurons and reduces nociceptive threshold in the hot plate test; these effects were abolished by pre-treatment with the GPR55 antagonist ML-193 (Deliu 2015). Using the chronic constriction injury (CCI) model of neuropathic pain it has been demonstrated that blockade of GPR55 activation in PAG neurons can restore and drive a descending control system to mitigate neuropathic pain in rodents (Armin 2021). Similarly, the GPR55 antagonist CID16020046 significantly reduced second phase formalin-evoked nociceptive behaviour in rats (Okine 2020). Taken together these data clearly suggest that the manipulation of GPR55 has therapeutic potential in the treatment of mechanical, inflammatory, and neuropathic pain.

### Depression

The ventral striatum and its role in mood, reward and motivation processing and behaviour has been the focus of significant research (Francis & Lobo 2017). The medium spiny neurons (MSNs) of the striatum are the main projection neurons in the ventral and dorsal striatum which can bidirectionally control reward and aversive stimuli and integrate these into emotional processing (Soares-Cunha 2020). Long term disruption in the reward circuitry and hyperactivity of aversive responsiveness have been suggested to mediate the motivation and emotional behavioural symptoms of major depression (Knowland & Lim 2018). Thus, approaches and targets which are able to modulate these aberrant neurocircuits have been suggested as therapeutics for major depression.

The significant expression of GPR55 in the striatum suggests an important role in this region. GPR55 is selectively expressed in the MSNs of both the dorsal and ventral striatum (Martinez-Pinilla 2014, suggesting a specific role in the modulation of the neurocircuitry implicated in mood. GPR55 gene expression was significantly lower in the dorsal lateral prefrontal cortex of suicide cases (Garcia-Gutierrez 2018). This is further supported by observations with GPR55 modulators in animal models of depression. Specifically, the GPR55 agonist O-1602 reversed symptoms of depression in rats treated with corticosterone using the forced swim behavioural test (Wrobel 2020).

Taken together, these data demonstrate that activation of GPR55 is likely to be of therapeutic utility in depression.

### Alzheimer's Disease (AD)

A recent study by Xiang (2022) has demonstrated a benefit of GPR55 activation in the Aβ₁₋₄₂ intracerebroventricular injection model of Alzheimer's Disease. Injection of GPR55 agonist O-1602 (Ryberg 2007) improved Aβ₁₋₄₂-induced memory deficits in behavioural models and these observations were associated with a decrease in measures of neurotoxicity such as catalase levels and the degree of cellular apoptosis (Xiang 2022). Furthermore, expression of GPR55 has been demonstrated to change in rodent models of Alzheimer's Disease (Medina-Vera 2020, Xiang 2022). These recent studies are suggestive that modulation of GPR55 activity could be therapeutically beneficial for Alzheimer's Disease.

### Neuroprotection

Neurotoxicity is a key driver of neurodegenerative diseases that can be abrogated by the action of the endocannabinoid system (Lowe 2021). As a pharmacological target of endocannabinoids which is expressed in brain regions susceptible in neurodegenerative diseases, the neuroprotective role of GPR55 has been explored in a range of neurotoxicity models. In a rat global cerebral ischaemia model, hippocampal neuronal loss (assessed 7 days post-ischaemic insult) was reversed by systemic dosing of LPI, the endogenous ligand of GPR55 (Blondeau 2002). In primary mouse cerebellar granule cells studied in vitro, treatment with LPI fully reversed glutamate-induced cell death (Blondeau 2002). Similarly, rat hippocampal granule cells and organotypic slices were protected from NMDA excitotoxicity by treatment with LPI, an effect which was reversed by siRNA-mediated knockdown of GPR55 (Kallendrusch 2013). In the SH-SY5Y neuronal cell model, overexpression of GPR55 receptors is protective against the neurotoxic agent MPP⁺ (Martinez-Pinilla 2019). Furthermore, GPR55 activation is neuroprotective in human and mouse neural stem cell (NSC) neurogenesis models (Hill 2019). Treatment of human NSCs with GPR55 agonist ML184 (Kotsikorou 2011) provided significant protection against IL-1β-induced reductions in neurogenesis; the protective effect of GPR55 agonism was reversed by co-application of ML-193, a GPR55 antagonist (Heynen-Genel 2010). Treatment of mouse NSCs with GPR55 agonist O-1602 (Ryberg 2007) promoted neurogenesis and this was reversed by GPR55 antagonist CID16020046 (Kargl 2013). These findings translated in vivo to increased neurogenesis following O-1602 treatment and protection against chronic inflammation; the positive effects of O-1602 treatment were absent in mice with genetic ablation of GPR55 (Hill 2019). Together these studies evidence a protective role for GPR55 activation in neurotoxicity and indicate a therapeutic benefit of GPR55 activation in slowing the progression of neurodegenerative diseases.

### REFERENCES

Armin 2021 DOI: 10.1016/j.bbr.2021.113248
Aso 2020 DOI: 10.1089/can.2019.0005
Baker 2006 DOI: 10.1016/j.tips.2005.11.003
Bjursell 2016 DOI: 10.1371/journal.pone.0167965
Blondeau 2002 DOI: 10.1097/00004647-200207000-00007
Brown 2007 DOI: 10.1038/sj.bjp.0707481
Brown 2011 DOI: 10.1124/jpet.110.172650
Burgaz 2021 DOI: 10.3390/molecules26247643
Celorrio 2017 DOI: 10.1016/j.neuropharm.2017.08.017
Deliu 2015 DOI: 10.1124/mol.115.099333
Devinsky 2017 DOI: 10.1056/nejmoa1611618
Devinsky 2018 DOI: 10.1056/nejmoa1714631
Do Val-da Silva 2017 DOI: 10.3389/fphar.2017.00131
Fatemi 2021 DOI: 10.1017/neu.2020.30
Francis & Lobo 2017 DOI: 10.1016/j.biopsych.2016.09.007
Garcia-Gutierrez 2018 DOI: 10.1007/s13311-018-0610-y
Henstridge 2009 DOI: 10.1096/fj.08-108670
Henstridge 2011 DOI: 10.1210/me.2011-1197
Heynen-Genel 2010 Bookshelf ID: NBK66153, PMID: 22091481
Hill 2019 DOI: 10.1016/j.bbi.2018.11.017
Izquierdo 1973 DOI: 10.1007/BF00413961
Kallendrusch 2013 DOI: 10.1002/glia.22560
Kaplan 2017 DOI: 10.1073/pnas.1711351114
Kargl 2013 DOI: 10.1124/ipet.113.204180
Khan 2018 DOI: 10.1111/bph.14202
Klein 2017 DOI: 10.1007/s11064-017-2287-8
Knowland & Lim 2018 DOI: 10.1016/j.pbb.2017.12.010
Kotsikorou 2011 DOI: 10.1021/bi200010k
Lauckner 2008 DOI: 10.1073/pnas.0711278105
Lowe 2021 DOI: 10.3390/ijms22179472
Marichal-Cancino 2017 DOI: 10.2174/1570159X14666160729155441
Marsicano 2003 DOI: 10.1126/science.1088208
Martínez-Pinilla 2019 DOI: 10.1007/s12035-019-1495-4
Martínez-Pinilla 2014 DOI: 10.1016/j.expneurol.2014.06.017
Medina-Vera 2020 DOI: 10.3390/biology9110377
Noronha 2017 DOI: 10.1016/j.bbr.2016.08.042
Oka 2007 DOI: 10.1016/j.bbrc.2007.08.078
Oka 2009 DOI: 10.1093/jb/mvn136
Okine 2020 DOI: 10.1016/j.neuroscience.2020.07.013
Rahimi 2015 DOI: 10.1111/fcp.12099
Regard 2008 DOI: 10.1016/j.cell.2008.08.040
Romero-Zerbo 2017 DOI: 10.1038/s41598-017-03292-w
Rosenberg 2018 Abstract: https://www.aesnet.org/abstractslisting/cannabidiol-(cbd)-exerts-antiepileptic-properties-by-targeting-the-lpi-gpr55-lipid-signaling-system-potentiated-by-seizures
Ruehle 2012 DOI: 10.1177/0269881111408958
Ryberg 2007 DOI: 10.1038/sj.bjp.0707460
Sharma 2013 DOI: 10.1038/ijo.2012.48
Shi 2017 DOI: 10.1186/s13041-017-0318-7
Soares-Cunha 2020 DOI: 10.1038/s41380-019-0484-3
Southern 2013 DOI: 10.1177/1087057113475480
Staton 2008 DOI: 10.1016/j.pain.2008.04.006
Sylantyev 2013 DOI: 10.1073/pnas.1211204110
Thiele 2018 DOI: 10.1016/S0140-6736 (18) 30136-3
Vázquez-León 2021 DOI: 10.1016/j.neulet.2021.136218
Wallace 2003 DOI: 10.1124/jpet.103.051920
Wrobel 2020 DOI: 10.3389/fphar.2020.01002
Wu 2013 DOI: 10.1371/journal.pone.0060314
Xiang 2022 DOI: 10.1016/j.pnpbp.2021.110423
Yin 2019 DOI: 10.1038/s41401-018-0051-5

There is a need for treatment of the above diseases and conditions and others described herein with compounds that are GPR55 modulators. The present invention provides modulators of GPR55.

### SUMMARY OF THE INVENTION

A first aspect of the present invention provides a compound of formula (I):

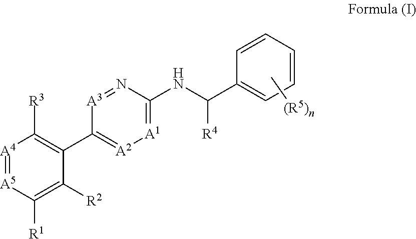

or a pharmaceutically acceptable salt, solvate or prodrug thereof, wherein:
A¹ is CH, CF or N;
A² is CH, CF or N;
A³ is CH, CMe, CCF₃ or N;
A⁴ is CH or N;
A⁵ is CH or N;
R¹ is halo, C₁-C₅ alkyl, C₂-C₅ alkenyl, C₂-C₅ alkynyl, C₃-C₅ cycloalkyl, —O(C₁-C₅ alkyl), —NH(C₁-C₃ alkyl), —N(C₁-C₃ alkyl)₂, —SO(C₁-C₃ alkyl), —SO₂(C₁-C₃ alkyl), —S(O)(NH)(C₁-C₃ alkyl) or —S(O)(N(C₁-C₃ alkyl))(C₁-C₃ alkyl), wherein any alkyl, alkenyl, alkynyl and cycloalkyl group is optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂;
or A⁵, R¹ and the carbon atom to which they are attached together form a 5-membered heterocyclic group containing 1 or 2 heteroatoms independently selected from N, O or S, wherein the heterocyclic group may contain an additional double bond, and wherein the heterocyclic group is optionally substituted with one or more substituents independently selected from halo, oxo (═O) or C₁-C₃ alkyl, wherein the C₁-C₃ alkyl is optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂;
R² is hydrogen or methyl;
R³ is hydrogen or methyl;
R⁴ is methyl;
R⁵ is independently halo, cyano, methyl or methoxy;
or, when a group R⁵ is in the ortho-position, R⁴ and R⁵ may together form a —CH₂CH₂— or —CH₂CH₂CH₂— group; and
n is 0, 1 or 2;
provided the compound is not:
5-(2-((1-phenylethyl)amino)pyrimidin-5-yl)-1,3-dihydro-2H-benzo[d]imidazol-2-one; or
5-(benzo[d][1,3]dioxol-5-yl)-N-(1-(4-fluorophenyl)ethyl)pyridin-2-amine.

In one embodiment, the compound of formula (I) is a compound of formula (Ia):

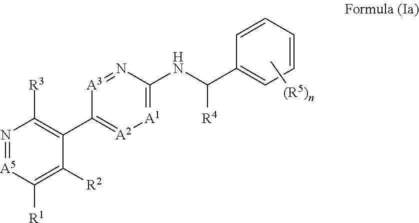

or a pharmaceutically acceptable salt, solvate or prodrug thereof, wherein:
A¹ is CH, CF or N;
A² is CH, CF or N;
A³ is CH, CMe, CCF₃ or N;
A⁵ is CH or N;
R¹ is halo, C₁-C₅ alkyl, C₂-C₅ alkenyl, C₂-C₅ alkynyl, C₃-C₅ cycloalkyl, —O(C₁-C₅ alkyl), —NH(C₁-C₃ alkyl), —N(C₁-C₃ alkyl)₂, —SO(C₁-C₃ alkyl), —SO₂(C₁-C₃ alkyl), —S(O)(NH)(C₁-C₃ alkyl) or —S(O)(N(C₁-C₃ alkyl))(C₁-C₃ alkyl), wherein any alkyl, alkenyl, alkynyl and cycloalkyl group is optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂;
R² is hydrogen or methyl;
R³ is hydrogen or methyl;
R⁴ is methyl;
R⁵ is independently halo, cyano, methyl or methoxy;
or, when a group R⁵ is in the ortho-position, R⁴ and R⁵ may together form a —CH₂CH₂— or —CH₂CH₂CH₂— group; and
n is 0, 1 or 2.

In compounds of formula (I) and (Ia), A⁵ is CH or N. In one embodiment, A⁵ is CH.

In compounds of formula (I) and (Ia), R¹ is halo (such as fluoro, chloro, bromo or iodo), C₁-C₅ alkyl, C₂-C₅ alkenyl, C₂-C₅ alkynyl, C₃-C₅ cycloalkyl, —O(C₁-C₅ alkyl), —NH(C₁-C₃ alkyl), —N(C₁-C₃ alkyl)₂, —SO(C₁-C₃ alkyl), —SO₂(C₁-C₃ alkyl), —S(O)(NH)(C₁-C₃ alkyl) or —S(O)(N(C₁-C₃ alkyl))(C₁-C₃ alkyl), wherein any alkyl, alkenyl, alkynyl and cycloalkyl group is optionally substituted with one or more (such as one, two, three, four or five) substituents independently selected from halo (such as fluoro, chloro, bromo or iodo), hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂.

In one embodiment, R¹ is halo (such as fluoro, chloro, bromo or iodo), C₁-C₃ alkyl, C₂-C₃ alkenyl, C₂-C₃ alkynyl, C₃-C₅ cycloalkyl, —O(C₁-C₃ alkyl), —NH(C₁-C₃ alkyl), —N(C₁-C₃ alkyl)₂, —SO(C₁-C₃ alkyl), —SO₂(C₁-C₃ alkyl), —S(O)(NH)(C₁-C₃ alkyl) or —S(O)(N(C₁-C₃ alkyl))(C₁-C₃ alkyl), wherein any alkyl, alkenyl, alkynyl and cycloalkyl group is optionally substituted with one or more (such as one, two, three, four or five) substituents independently selected from halo (such as fluoro, chloro, bromo or iodo), hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂.

In one embodiment, R¹ is fluoro, chloro, methyl, ethyl, vinyl, ethynyl, cyclopropyl, methoxy, ethoxy, —NHMe, —NMe₂, —SOMe, —SOEt, —SO₂Me, —SO₂Et, —S(O)(NH)Me or —S(O)(NMe)Me, each of which is optionally substituted with one, two, three or four substituents independently selected from fluoro, chloro, hydroxyl, —OMe, —SMe, —SOMe, —SO₂Me, —NHMe or —NMe₂.

In one embodiment, R¹ is chloro, methyl, ethyl, vinyl, ethynyl, cyclopropyl, methoxy, ethoxy, —NMe₂, —SOMe, —SOEt, —SO₂Me, —SO₂Et, —S(O)(NH)Me or —S(O)(NMe)Me, each of which is optionally substituted with one, two, three or four substituents independently selected from fluoro, hydroxyl or —NHMe.

In one embodiment, R¹ is ethyl substituted with two or three fluoro or R¹ is —SO₂Me.

In another embodiment, the compound of formula (I) is a compound of formula (Ib):

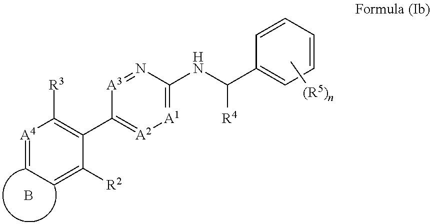

or a pharmaceutically acceptable salt, solvate or prodrug thereof, wherein:
A¹ is CH, CF or N;
A² is CH, CF or N;
A³ is CH, CMe, CCF₃ or N;
A⁴ is CH or N;
B is a 5-membered heterocyclic group containing 1 or 2 heteroatoms independently selected from N, O or S, wherein the heterocyclic group may contain an additional double bond, and wherein the heterocyclic group is optionally substituted with one or more substituents independently selected from halo, oxo (═O) or C₁-C₃ alkyl, wherein the C₁-C₃ alkyl is optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂;
R² is hydrogen or methyl;
R³ is hydrogen or methyl;
R⁴ is methyl;
R⁵ is independently halo, cyano, methyl or methoxy;
or, when a group R⁵ is in the ortho-position, R⁴ and R⁵ may together form a —CH₂CH₂— or —CH₂CH₂CH₂— group; and
n is 0, 1 or 2;
provided the compound is not:
5-(2-((1-phenylethyl)amino)pyrimidin-5-yl)-1,3-dihydro-2H-benzo[d]imidazol-2-one; or
5-(benzo[d][1,3]dioxol-5-yl)-N-(1-(4-fluorophenyl)ethyl)pyridin-2-amine.

In compounds of formula (I) and (Ib), A⁴ is CH or N.

In compounds of formula (I) and (Ib), in one embodiment, each of A¹, A², A³ and A⁴ is independently selected from CH or N, provided zero, one, two or three of A¹, A², A³ and A⁴ are N. In one embodiment, each of A¹, A², A³ and A⁴ is independently selected from CH or N, provided one or two of A¹, A², A³ and A⁴ are N.

In one embodiment, in a compound of formula (Ib), B is a ring represented by the group:

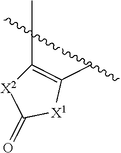

wherein:
X¹ is O, NH or NR⁷;
X² is O, NH, CF₂, CHF or CH₂; and
R⁷ is C₁-C₃ alkyl optionally substituted with one or more (such as one, two, three, four or five) substituents independently selected from halo (such as fluoro, chloro, bromo or iodo), hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂.

In one embodiment, X¹ is NR⁷.

In one embodiment, X² is O, NH or CF₂. In one embodiment, X² is O.

In one embodiment, R⁷ is C₁-C₃ alkyl optionally substituted with one, two or three substituents independently selected from fluoro, chloro, bromo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂.

In one embodiment, R⁷ is methyl or ethyl optionally substituted with one, two or three substituents independently selected from fluoro, chloro, hydroxyl, —OMe, —SMe, —SOMe, —SO₂Me, —NHMe or —NMe₂.

In one embodiment, R⁷ is methyl or ethyl optionally substituted with one, two or three substituents independently selected from fluoro, hydroxyl, —OMe, —SMe, —SO₂Me or —NMe₂.

In one embodiment, R⁷ is methyl optionally substituted with one, two or three substituents independently selected from fluoro or —OMe.

In another embodiment, in a compound of formula (Ib), B is a 5-membered heteroaryl group containing 1 or 2 heteroatoms independently selected from N, O or S, wherein the heteroaryl group is optionally substituted with one or two substituents independently selected from halo (such as fluoro, chloro, bromo or iodo) or C₁-C₃ alkyl, wherein the C₁-C₃ alkyl is optionally substituted with one or more (such as one, two, three, four or five) substituents independently selected from halo (such as fluoro, chloro, bromo or iodo), hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂.

In one embodiment, B is a 5-membered heteroaryl group containing 1 or 2 heteroatoms independently selected from N or O, wherein the heteroaryl group is optionally substituted with one or two substituents independently selected from fluoro, chloro, bromo or C₁-C₃ alkyl, wherein the C₁-C₃ alkyl is optionally substituted with one or more (such as one, two, three, four or five) substituents independently selected from fluoro, chloro, bromo, hydroxyl, —OMe, —SMe, —SOMe, —SO₂Me, —NHMe or —NMe₂.

In one embodiment, B is a pyrazolyl, imidazolyl, oxazolyl or isoxazolyl group, each of which is optionally substituted with fluoro, chloro or C₁-C₃ alkyl.

In one embodiment, B is an imidazolyl or oxazolyl group, each of which is optionally substituted with fluoro, chloro or methyl.

In one embodiment, B is an oxazolyl, 1-methyl-imidazolyl, or 3-methyl-imidazolyl group.

In one embodiment, B is an oxazolyl group.

In compounds of formula (I), (Ia) and (Ib), A¹ is CH, CF or N. In one embodiment, A¹ is CH or N.

In compounds of formula (I), (Ia) and (Ib), A² is CH, CF or N.

In compounds of formula (I), (Ia) and (Ib), A³ is CH, CMe, CCF₃ or N. In one embodiment, A³ is CH, CMe or N. In one embodiment, A³ is CH or N.

In compounds of formula (I), (Ia) and (Ib), in one embodiment, at least one of A¹, A² and A³ is N or CF. In one embodiment, at least one of A¹ and A² is N or CF.

In compounds of formula (I), (Ia) and (Ib), R² is hydrogen or methyl. In one embodiment, R² is hydrogen.

In compounds of formula (I), (Ia) and (Ib), R³ is hydrogen or methyl. In one embodiment, R³ is hydrogen.

In compounds of formula (I), (Ia) and (Ib), R⁴ is methyl; R⁵ is independently halo (such as fluoro, chloro, bromo or iodo), cyano, methyl or methoxy; or, when a group R⁵ is in the ortho-position, R⁴ and R⁵ may together form a —CH₂CH₂— or —CH₂CH₂CH₂— group.

In one embodiment, R⁵ is independently fluoro, chloro, bromo, cyano, methyl or methoxy. In one embodiment, R⁵ is independently fluoro, chloro, cyano, methyl or methoxy. In one embodiment, R⁵ is independently fluoro, chloro or methyl. In one embodiment, R⁵ is fluoro.

In one embodiment, when a group R⁵ is in the ortho-position, R⁴ and R⁵ together form a —CH₂CH₂— group.

In compounds of formula (I), (Ia) and (Ib), n is 0, 1 or 2. In one embodiment, n is 0. In a preferred embodiment, n is 1. In another embodiment, n is 2. When n is 2, the two substituents R⁵ can be in the 2,3-, 2,4-, 2,5-, 2,6-, 3,4- or 3,5-positions. In one embodiment, n is 2, and the two substituents R⁵ are in the 3,4- or 2,4-positions. In another embodiment, n is 2, and the two substituents R⁵ are in the 3,4-positions.

In one specific embodiment of the first aspect, the present invention provides a compound of formula (Ia), or a pharmaceutically acceptable salt, solvate or prodrug thereof, wherein:
A¹ is CH or N;
A² is CH, CF or N;
A³ is CH, CMe or N;
A⁵ is CH or N;
R¹ is fluoro, chloro, methyl, ethyl, vinyl, ethynyl, cyclopropyl, methoxy, ethoxy, —NHMe, —NMe₂, —SOMe, —SOEt, —SO₂Me, —SO₂Et, —S(O)(NH)Me or —S(O)(NMe)Me, each of which is optionally substituted with one, two, three or four substituents independently selected from fluoro, chloro, hydroxyl, —OMe, —SMe, —SOMe, —SO₂Me, —NHMe or —NMe₂;
R² is hydrogen or methyl;
R³ is hydrogen or methyl;
R⁴ is methyl;
R⁵ is independently halo, cyano, methyl or methoxy;
or, when a group R⁵ is in the ortho-position, R⁴ and R⁵ may together form a —CH₂CH₂— group; and
n is 0, 1 or 2.

In another specific embodiment of the first aspect, the present invention provides a compound of formula (Ib), or a pharmaceutically acceptable salt, solvate or prodrug thereof, wherein:
each of A¹, A², A³ and A⁴ is independently selected from CH or N, provided zero, one, two or three of A¹, A², A³ and A⁴ are N;
B is a pyrazolyl, imidazolyl, oxazolyl or isoxazolyl group, each of which is optionally substituted with fluoro, chloro or C₁-C₃ alkyl;
R² is hydrogen;
R³ is hydrogen;
R⁴ is methyl;
R⁵ is independently halo, cyano, methyl or methoxy; and
n is 0, 1 or 2.

In another specific embodiment of the first aspect, the present invention provides a compound of formula (Ib′), or a pharmaceutically acceptable salt, solvate or prodrug thereof, wherein:

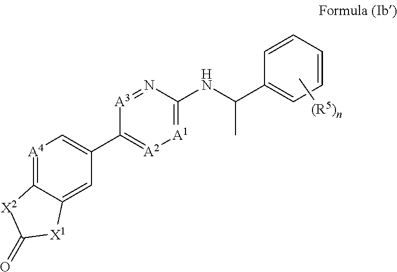

each of A¹, A², A³ and A⁴ is independently selected from CH or N, provided zero, one, two or three of A¹, A², A³ and A⁴ are N;
X¹ is O, NH or NR⁷;
X² is O, NH or CF₂;
R⁵ is independently halo, cyano, methyl or methoxy;
R⁷ is methyl or ethyl optionally substituted with one, two or three substituents independently selected from fluoro, chloro, hydroxyl, —OMe, —SMe, —SOMe, —SO₂Me, —NHMe or —NMe₂; and
n is 0, 1 or 2;
provided the compound is not 5-(2-((1-phenylethyl)amino)pyrimidin-5-yl)-1,3-dihydro-2H-benzo[d]imidazol-2-one.

A second aspect of the present invention provides a compound selected from:
N-(1-(4-fluorophenyl)ethyl)-5′-methyl-[3,3′-bipyridin]-6-amine;
(R)-N-(1-(3,4-difluorophenyl)ethyl)-5′-methyl-[3,3′-bipyridin]-6-amine;
(R)-N-(1-(4-fluorophenyl)ethyl)-5′-methyl-[3,3′-bipyridin]-6-amine;
(R)-5′-ethyl-N-(1-(4-fluorophenyl)ethyl)-[3,3′-bipyridin]-6-amine;
(R)-5′-cyclopropyl-N-(1-(4-fluorophenyl)ethyl)-[3,3′-bipyridin]-6-amine;
(R)-N-(1-(4-fluorophenyl)ethyl)-5′-methoxy-[3,3′-bipyridin]-6-amine;
(R)-N-(1-(4-fluorophenyl)ethyl)-5′-(methylsulfonyl)-[3,3′-bipyridin]-6-amine;
(R)-N-(1-(4-fluorophenyl)ethyl)-5′-(trifluoromethyl)-[3,3′-bipyridin]-6-amine;
(R)-5′-chloro-N-(1-(4-fluorophenyl)ethyl)-[3,3′-bipyridin]-6-amine;
(R)-N-(1-(4-fluorophenyl)ethyl)-2′,5′-dimethyl-[3,3′-bipyridin]-6-amine;
(R)-N-(1-(4-fluorophenyl)ethyl)-4′,5′-dimethyl-[3,3′-bipyridin]-6-amine;
(R)-N6′-(1-(4-fluorophenyl)ethyl)-N5,N5-dimethyl-[3,3′-bipyridine]-5,6′-diamine;
5′-(ethylsulfinyl)-N-((R)-1-(4-fluorophenyl)ethyl)-[3,3′-bipyridin]-6-amine;
N-((R)-1-(4-fluorophenyl)ethyl)-5′-(methylsulfinyl)-[3,3′-bipyridin]-6-amine;
(R)-5′-(ethylsulfonyl)-N-(1-(4-fluorophenyl)ethyl)-[3,3′-bipyridin]-6-amine;
1-(6′-(((R)-1-(4-fluorophenyl)ethyl)amino)-[3,3′-bipyridin]-5-yl)ethan-1-ol;
(R)-5-(5-ethylpyridin-3-yl)-N-(1-(4-fluorophenyl)ethyl)pyrazin-2-amine;
(R)-N-(1-(4-fluorophenyl)ethyl)-5-(5-(methylsulfonyl)pyridin-3-yl)pyrazin-2-amine;
(R)-5′-ethyl-N-(1-(2-fluorophenyl)ethyl)-[3,3′-bipyridin]-6-amine;
(R)-N-(1-(4-fluorophenyl)ethyl)-5′-vinyl-[3,3′-bipyridin]-6-amine;
(R)-N-(2,3-dihydro-1H-inden-1-yl)-5′-ethyl-[3,3′-bipyridin]-6-amine;
(R)-N-(1-(4-fluorophenyl)ethyl)-2-methyl-5′-(methylsulfonyl)-[3,3′-bipyridin]-6-amine;
N-((R)-1-(4-fluorophenyl)ethyl)-5-(5-(methylsulfinyl)pyridin-3-yl)pyrazin-2-amine;
5-(5-(ethylsulfinyl)pyridin-3-yl)-N-((R)-1-(4-fluorophenyl)ethyl)pyrazin-2-amine;
(R)-N-(1-(4-fluorophenyl)ethyl)-5-(5-(methylsulfonyl)pyridin-3-yl)pyrimidin-2-amine;
(R)-6-(5-((1-(4-fluorophenyl)ethyl)amino)pyrazin-2-yl)-1-methyloxazolo[5,4-b]pyridin-2(1H)-one;
(6′-(((R)-1-(4-fluorophenyl)ethyl)amino)-[3,3′-bipyridin]-5-yl)(imino)(methyl)-λ⁶-sulfanone;
(R)-N-(1-(4-fluorophenyl)ethyl)-6-(5-(methylsulfonyl)pyridin-3-yl)-1,2,4-triazin-3-amine;
4-fluoro-N-(1-(4-fluorophenyl)ethyl)-5′-(methylsulfonyl)-[3,3′-bipyridin]-6-amine;
(6′-(((R)-1-(4-fluorophenyl)ethyl)amino)-[3,3′-bipyridin]-5-yl)(methyl)(methylimino)-λ6-sulfanone;
(R)-5-(5-(2,2-difluoroethyl)pyridin-3-yl)-N-(1-(4-fluorophenyl)ethyl)pyrazin-2-amine;
2,2,2-trifluoro-1-(5-(5-(((R)-1-(4-fluorophenyl)ethyl)amino)pyrazin-2-yl)pyridin-3-yl)ethan-1-ol;
(R)-N-(1-(4-fluorophenyl)ethyl)-5-(5-vinylpyridin-3-yl)pyrazin-2-amine;
(R)-5-(5-(2,2-difluorovinyl)pyridin-3-yl)-N-(1-(4-fluorophenyl)ethyl)pyrazin-2-amine;
(R)-2-(5-(5-((1-(4-fluorophenyl)ethyl)amino)pyrazin-2-yl)pyridin-3-yl)ethan-1-ol;
(R)-N-(2,3-dihydro-1H-inden-1-yl)-5-(5-(methylsulfonyl)pyridin-3-yl)pyrazin-2-amine;
(R)-N-(1-(4-fluorophenyl)ethyl)-5-(5-(methylamino)methylpyridin-3-yl)pyrazin-2-amine;
N-(5-fluoro-2,3-dihydro-1H-inden-1-yl)-5′-(methylsulfonyl)-[3,3′-bipyridin]-6-amine;
(R)-5-(5-(difluoromethoxy)pyridin-3-yl)-N-(1-(4-fluorophenyl)ethyl)pyrazin-2-amine;
(R)-6-(5-(2,2-difluorovinyl)pyridin-3-yl)-N-(1-(4-fluorophenyl)ethyl)-1,2,4-triazin-3-amine;
(R)-(5-(3-((1-(4-fluorophenyl)ethyl)amino)-1,2,4-triazin-6-yl)pyridin-3-yl)methanol;
(R)-N-(1-(4-fluorophenyl)ethyl)-5-(5-(2,2,2-trifluoroethoxy)pyridin-3-yl)pyrazin-2-amine;
(R)-N-(1-(4-fluorophenyl)ethyl)-5-(5-(trifluoromethoxy)pyridin-3-yl)pyrazin-2-amine;
(R)-N-(1-(4-fluorophenyl)ethyl)-5-(6-vinylpyridazin-4-yl)pyrazin-2-amine;
1-(5-(5-(((R)-2,3-dihydro-1H-inden-1-yl)amino)pyrazin-2-yl)pyridin-3-yl)-2,2-difluoroethan-1-ol;
N-(1-(4-chlorophenyl)ethyl)-5′-methyl-[3,3′-bipyridin]-6-amine;
(R)-5′-ethyl-N-(1-(4-methoxyphenyl)ethyl)-[3,3′-bipyridin]-6-amine;
(R)-5′-ethyl-N-(1-(3-fluorophenyl)ethyl)-[3,3′-bipyridin]-6-amine;
(R)-5′-ethyl-N-(1-phenylethyl)-[3,3′-bipyridin]-6-amine;
(R)-5′-ethyl-N-(1-(p-tolyl)ethyl)-[3,3′-bipyridin]-6-amine;
(R)-5′-ethynyl-N-(1-(4-fluorophenyl)ethyl)-[3,3′-bipyridin]-6-amine;
(R)-5-(5-(2-fluoroethyl)pyridin-3-yl)-N-(1-(4-fluorophenyl)ethyl)pyrazin-2-amine;
(R)-N-(1-(4-fluorophenyl)ethyl)-5-(5-(2,2,2-trifluoroethyl)pyridin-3-yl)pyrazin-2-amine;
2,2-difluoro-1-(5-(5-(((R)-1-(4-fluorophenyl)ethyl)amino)pyrazin-2-yl)pyridin-3-yl)ethan-1-ol;
(R)-N-(1-(4-fluorophenyl)ethyl)-6-(5-(2,2,2-trifluoroethyl)pyridin-3-yl)-1,2,4-triazin-3-amine;
(R)-N-(1-(4-fluorophenyl)ethyl)-3-(5-(2,2,2-trifluoroethyl)pyridin-3-yl)-1,2,4-triazin-6-amine;
(R)-N-(1-(4-fluorophenyl)ethyl)-5-(6-(2,2,2-trifluoroethyl)pyridazin-4-yl)pyrazin-2-amine;
2,2,2-trifluoro-1-(5-(5-(((R)-1-(4-fluorophenyl)ethyl)amino)pyrazin-2-yl)pyridazin-3-yl)ethan-1-ol;
(R)-5-(6-((1-(4-fluorophenyl)ethyl)amino)pyridin-3-yl)benzo[d]oxazol-2(3H)-one;
(R)-5-(6-((1-(4-fluorophenyl)ethyl)amino)pyridin-3-yl)-3-methylbenzo[d]oxazol-2(3H)-one;
(R)-6-(6-((1-(4-fluorophenyl)ethyl)amino)pyridin-3-yl)benzo[d]oxazol-2(3H)-one;
(R)-5-(5-((1-(4-fluorophenyl)ethyl)amino)pyrazin-2-yl)-3-methylbenzo[d]oxazol-2(3H)-one;
(R)-5-(2-((1-(4-fluorophenyl)ethyl)amino)pyrimidin-5-yl)-3-methylbenzo[d]oxazol-2(3H)-one;
(R)-5-(3-((1-(4-fluorophenyl)ethyl)amino)-1,2,4-triazin-6-yl)-3-methylbenzo[d]oxazol-2(3H)-one;
(R)-5-(3-((1-(4-fluorophenyl)ethyl)amino)-1,2,4-triazin-6-yl)-3-(2-hydroxyethyl)benzo[d]oxazol-2(3H)-one;
(R)-5-(3-((1-(4-fluorophenyl)ethyl)amino)-1,2,4-triazin-6-yl)-3-(methoxymethyl)benzo[d]oxazol-2(3H)-one;
(R)-3-ethyl-5-(3-((1-(4-fluorophenyl)ethyl)amino)-1,2,4-triazin-6-yl)benzo[d]oxazol-2(3H)-one;
(R)-3-(difluoromethyl)-5-(3-((1-(4-fluorophenyl)ethyl)amino)-1,2,4-triazin-6-yl)benzo[d]oxazol-2(3H)-one;
(R)-5-(3-((1-(4-fluorophenyl)ethyl)amino)-1,2,4-triazin-6-yl)benzo[d]oxazol-2(3H)-one;
(R)-5-(3-((1-(4-fluorophenyl)ethyl)amino)-1,2,4-triazin-6-yl)-3-((methylthio)methyl)benzo[d]oxazol-2(3H)-one;
(R)-3-(2-(dimethylamino)ethyl)-5-(3-((1-(4-fluorophenyl)ethyl)amino)-1,2,4-triazin-6-yl)benzo[d]oxazol-2(3H)-one;
(R)-5-(3-((1-(4-fluorophenyl)ethyl)amino)-1,2,4-triazin-6-yl)-3-((methylsulfonyl)methyl)benzo[d]oxazol-2(3H)-one;
(R)-5-(3-((1-(4-chlorophenyl)ethyl)amino)-1,2,4-triazin-6-yl)-3-methylbenzo[d]oxazol-2(3H)-one;
4-(1-((6-(3-methyl-2-oxo-2,3-dihydrobenzo[d]oxazol-5-yl)-1,2,4-triazin-3-yl)amino)ethyl)benzonitrile;
(R)-5-(3-((1-(2,4-difluorophenyl)ethyl)amino)-1,2,4-triazin-6-yl)-3-methylbenzo[d]oxazol-2(3H)-one;
(R)-5-(3-((1-(3,4-difluorophenyl)ethyl)amino)-1,2,4-triazin-6-yl)-3-methylbenzo[d]oxazol-2(3H)-one;
5-(3-((1-(4-fluoro-3-methylphenyl)ethyl)amino)-1,2,4-triazin-6-yl)-3-methylbenzo[d]oxazol-2(3H)-one;
5-(3-((1-(3,4-dichlorophenyl)ethyl)amino)-1,2,4-triazin-6-yl)-3-methylbenzo[d]oxazol-2(3H)-one;
(R)-3,3-difluoro-6-(6-((1-(4-fluorophenyl)ethyl)amino)pyridin-3-yl)-1-methylindolin-2-one;
(R)-6-(benzo[d]oxazol-5-yl)-N-(1-(4-fluorophenyl)ethyl)-1,2,4-triazin-3-amine;
(R)-N-(1-(4-fluorophenyl)ethyl)-6-(1-methyl-1H-benzo[d]imidazol-6-yl)-1,2,4-triazin-3-amine;
(R)-N-(1-(4-fluorophenyl)ethyl)-6-(1-methyl-1H-imidazo[4,5-b]pyridin-6-yl)-1,2,4-triazin-3-amine;
(R)-N-(1-(4-fluorophenyl)ethyl)-6-(3-methyl-3H-imidazo[4,5-b]pyridin-6-yl)-1,2,4-triazin-3-amine;
   or an enantiomer of any of the foregoing;
   or a pharmaceutically acceptable salt, solvate or prodrug of any of the foregoing.

Preferably the compound of the first or second aspect has a chemical purity of 95% or more, preferably 96% or more, preferably 97% or more, preferably 98% or more, preferably 99% or more, preferably 99.5% or more, preferably 99.8% or more, preferably 99.9% or more, as measured by HPLC or UPLC.

Preferably the compound of the first or second aspect has a stereochemical purity of 95% or more, preferably 96% or more, preferably 97% or more, preferably 98% or more, preferably 99% or more, preferably 99.5% or more, preferably 99.8% or more, preferably 99.9% or more, as measured by XRPD or SFC.

A third aspect of the present invention provides a process for the preparation of a compound of formula (I), (Ia) or (Ib), or a pharmaceutically acceptable salt, solvate or prodrug thereof, according to the first or second aspect of the present invention, wherein the process comprises:
(a) reacting a compound of formula (II) or a salt thereof, with a compound of formula (III) or a salt thereof:

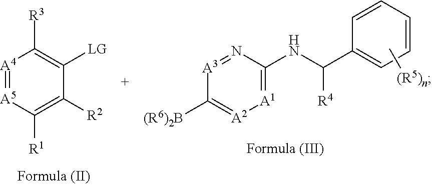

or
(b) reacting a compound of formula (IV) or a salt thereof, with an amine of formula (V) or a salt thereof:

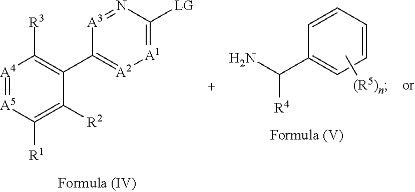

or
(c) reacting a compound of formula (VI) or a salt thereof, with a compound of formula (VII) or a salt thereof:

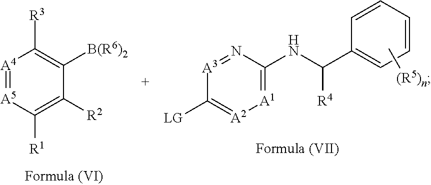

wherein:
R¹, R², R³, R⁴, R⁵, A¹, A², A³, A⁴ and A⁵ are as defined in the first aspect of the present invention or can be converted into such a group;
n is as defined in the first aspect of the present invention;
R⁶ is independently selected from hydroxyl, C₁-C₅ alkoxy or C₁-C₅ alkyl, or two R⁶ together with the boron to which they are attached form an optionally substituted 5- to 6-membered heterocyclic group; and
LG is a leaving group such as a halo (such as fluoro, chloro, bromo or iodo), a sulfate group (such as methyl sulfate), or a sulfonate group (such as mesylate, triflate or tosylate);
   and optionally thereafter carrying out one or more of the following procedures:
converting a compound of formula (I), (Ia) or (Ib) into another compound of formula (I), (Ia) or (Ib);
removing any protecting groups;
forming a pharmaceutically acceptable salt.

Examples of converting a compound of formula (I), (Ia) or (Ib) into another compound of formula (I), (Ia) or (Ib) are Examples 53, 55 and 57.

In one embodiment of the process of the present invention, a compound of formula (II) or a salt thereof, is reacted with a compound of formula (III) or a salt thereof:

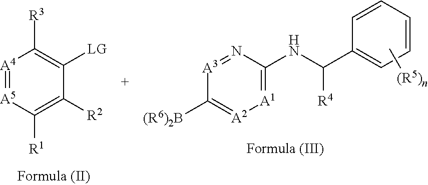

wherein R¹, R², R³, R⁴, R⁵, A¹, A², A³, A⁴ and A⁵ are as defined in the first aspect of the present invention or can be converted into such a group; n is as defined in the first aspect of the present invention; R⁶ is independently selected from hydroxyl, C₁-C₅ alkoxy or C₁-C₅ alkyl, or two R⁶ together with the boron to which they are attached form an optionally substituted 5- to 6-membered heterocyclic group; and LG is a leaving group such as fluoro, chloro, bromo, iodo, methyl sulfate, mesylate, triflate or tosylate. This process is depicted schematically in Scheme 1.

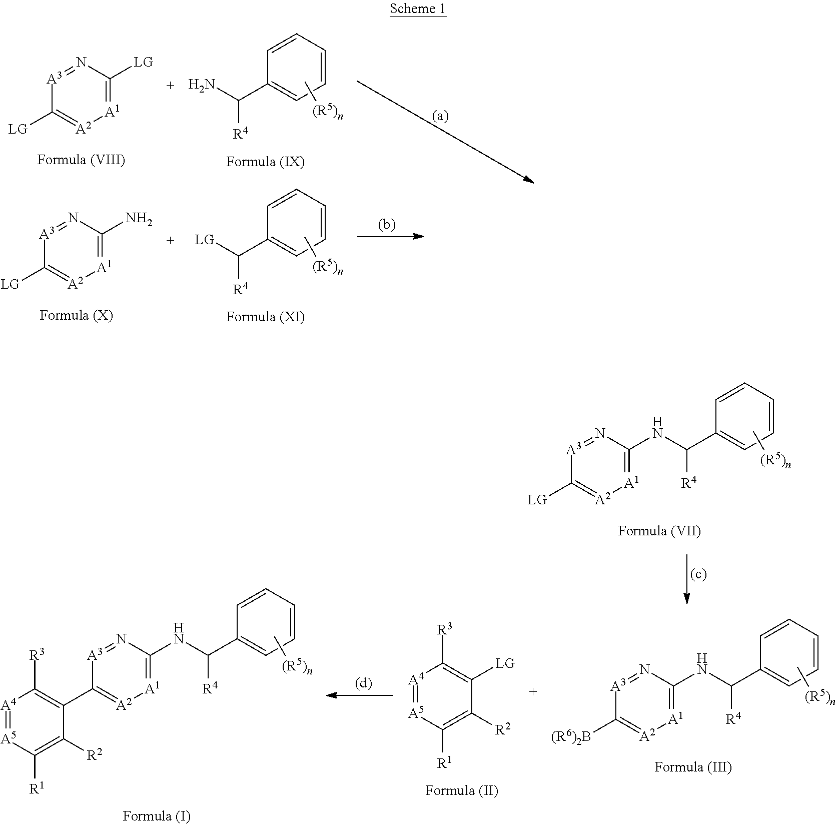

A compound of formula (III) can be prepared in a two-step process as shown in steps (a) and (c), or steps (b) and (c) of Scheme 1. In step (a), a compound of formula (VIII) or a salt thereof, is reacted with a compound of formula (IX) or a salt thereof, to provide a compound of formula (VII) or a salt thereof, wherein each LG is independently a leaving group such as fluoro, chloro, bromo, iodo, methyl sulfate, mesylate, triflate or tosylate. Each LG may be the same or different. The reaction is typically carried out in the presence of a base, such as triethylamine, K₂CO₃ or DIPEA, typically in a solvent such as DMSO, ethanol, dioxane or NMP. The reaction is typically carried out at a temperature of about 20-170° C. for about 2-72 hours.

The conversion achieved by step (a) can alternatively be achieved by reacting a compound of formula (VIII) or a salt thereof, with a compound of formula (IX) or a salt thereof, in the presence of a palladium catalyst such as Pd₂(dba)₃. This reaction is typically carried out in the presence of a base such as Cs₂CO₃, optionally with BINAP. The reaction is typically carried out in a solvent such as toluene and typically at a temperature of about 20-110° C. for about 2-16 hours.

In step (b) of Scheme 1, a compound of formula (X) or a salt thereof, is reacted with a compound of formula (XI) or a salt thereof, to provide a compound of formula (VII) or a salt thereof, wherein each LG is independently a leaving group such as fluoro, chloro, bromo, iodo, methyl sulfate, mesylate, triflate or tosylate. Each LG may be the same or different. The reaction is typically carried out in the presence of a base such as NaH, typically in a solvent such as DMF. The reaction is typically carried out at a temperature of about 0-25° C. for about 1-12 hours.

Step (c) of Scheme 1 may conveniently be carried out using a Miyaura-borylation reaction. In step (c), a compound of formula (VII) or a salt thereof, is reacted with a boron compound such as bis(pinacolato)diboron and a palladium catalyst such as PdCl₂(dppf) or Pd(OAc)₂ optionally with tricyclohexylphosphine. The reaction is typically carried out in the presence of a base such as KOAc and typically in a solvent such as dioxane. The reaction is typically carried out at a temperature of about 20-110° C. for about 2-72 hours.

Step (d) of Scheme 1 may conveniently be carried out by a Suzuki reaction. LG is a leaving group such as fluoro, chloro, bromo, iodo, methyl sulfate, mesylate, triflate or tosylate. R⁶ may be selected such that the compound of formula (III) is selected from:

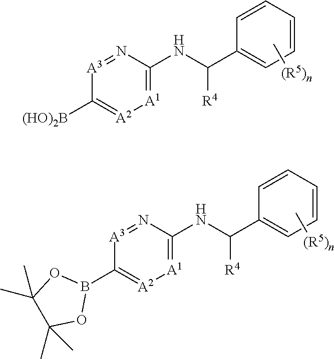

In step (d), a compound of formula (III) or a salt thereof, is reacted with a compound of formula (II) or a salt thereof, in the presence of a palladium catalyst such as PdCl₂(dppf). The reaction is typically carried out in the presence of a base such as K₂CO₃ and in a solvent such as dioxane or water or a mixture thereof. The reaction is typically carried out at a temperature of about 20-110° C. for about 2-20 hours.

In another embodiment of the process of the present invention, a compound of formula (IV) or a salt thereof, is reacted with an amine of formula (V) or a salt thereof:

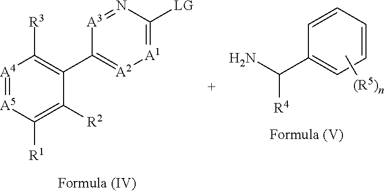

wherein R¹, R², R³, R⁴, R⁵, A¹, A², A³, A⁴ and A⁵ are as defined in the first aspect of the present invention or can be converted into such a group; n is as defined in the first aspect of the present invention; and LG is a leaving group such as fluoro, chloro, bromo, iodo, methyl sulfate, mesylate, triflate or tosylate. This process is depicted schematically in Scheme 2.

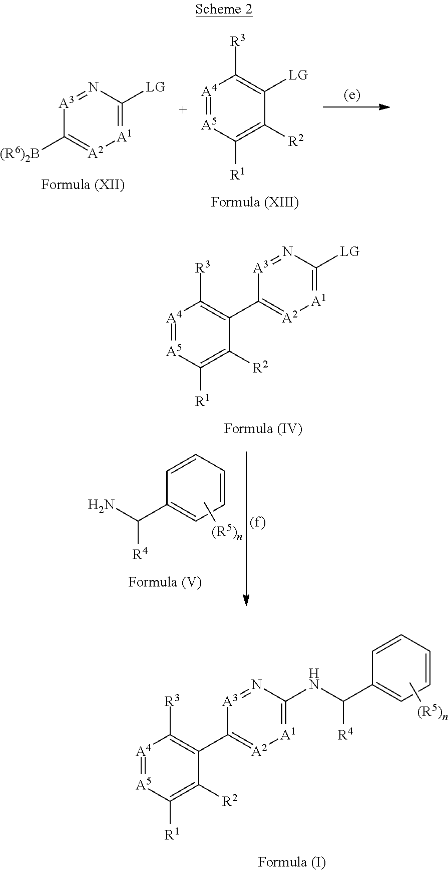

Step (e) of Scheme 2 may conveniently be carried out by a Suzuki reaction. Each LG is independently a leaving group such as fluoro, chloro, bromo, iodo, methyl sulfate, mesylate, triflate or tosylate. Each LG may be the same or different. R⁶ is independently selected from hydroxyl, C₁-C₅ alkoxy or C₁-C₅ alkyl, or two R⁶ together with the boron to which they are attached form an optionally substituted 5- to 6-membered heterocyclic group. R⁶ may be selected such that the compound of formula (XII) is selected from:

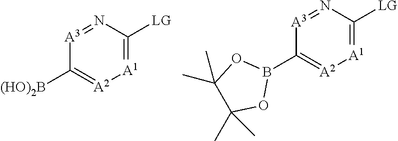

In step (e), a compound of formula (XII) or a salt thereof, is reacted with a compound of formula (XIII) or a salt thereof, in the presence of a palladium catalyst such as PdCl₂(dppf). The reaction is typically carried out in the presence of a base such as K₂CO₃ and in a solvent such as dioxane or water or a mixture thereof. The reaction is typically carried out at a temperature of about 20-110° C. for about 2-20 hours.

In step (f) of Scheme 2, a compound of formula (IV) or a salt thereof, is reacted with a compound of formula (V) or a salt thereof, to provide a compound of formula (I). The reaction is typically carried out in the presence of a palladium catalyst such as Pd₂(dba)₃.

The reaction is typically carried out in the presence of a base such as Cs₂CO₃, optionally with BINAP. The reaction is typically carried out in a solvent such as toluene and typically at a temperature of about 20-110° C. for about 2-16 hours.

The reaction of step (f) of a compound of formula (IV) or a salt thereof, with a compound of formula (V) or a salt thereof, can alternatively be carried out in the presence of a base, such as trimethylamine, K₂CO₃ or DIPEA, typically in a solvent such as DMSO, ethanol, dioxane or NMP. This reaction is typically carried out at a temperature of about 20-170° C. for about 2-72 hours.

In yet another embodiment of the process of the present invention, a compound of formula (VI) or a salt thereof, is reacted with a compound of formula (VII) or a salt thereof:

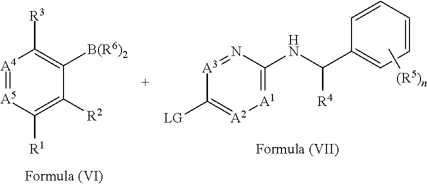

wherein R¹, R², R³, R⁴, R⁵, A¹, A², A³, A⁴ and A⁵ are as defined in the first aspect of the present invention or can be converted into such a group; n is as defined in the first aspect of the present invention; R⁶ is independently selected from hydroxyl, C₁-C₅ alkoxy or C₁-C₅ alkyl, or two R⁶ together with the boron to which they are attached form an optionally substituted 5- to 6-membered heterocyclic group; and LG is a leaving group such as fluoro, chloro, bromo, iodo, methyl sulfate, mesylate, triflate or tosylate. This process is depicted schematically in Scheme 3.

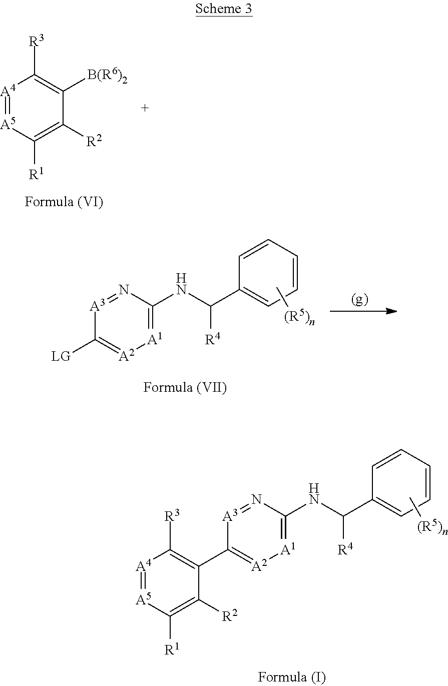

A compound of formula (VII) can be prepared as described in Scheme 1. Step (g) may conveniently be carried out by a Suzuki reaction. R⁶ may be selected such that the compound of formula (VI) is selected from:

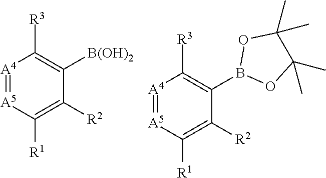

In step (g), a compound of formula (VI) or a salt thereof, is reacted with a compound of formula (VII) or a salt thereof, in the presence of a palladium catalyst such as PdCl₂(dppf). The reaction is typically carried out in the presence of a base such as K₂CO₃ and in a solvent such as dioxane or water or a mixture thereof. The reaction is typically carried out at a temperature of about 20-110° C. for about 2-20 hours.

It will be appreciated by those skilled in the art that in the processes of the present invention certain functional groups such as phenol, alkynyl, hydroxy or amino groups in the reagents may need to be protected by protecting groups. Thus, the preparation of the compounds, salts, solvates and prodrugs of the present invention may involve, at an appropriate stage, the introduction and/or removal of one or more protecting groups. An example of the introduction and/or removal of one or more protecting groups is in Example 51.

The protection and deprotection of functional groups are described, for example, in ‘Protective Groups in Organic Chemistry’, edited by J. W. F. McOmie, Plenum Press (1973); ‘Greene's Protective Groups in Organic Synthesis’, 4th edition, T. W. Greene and P. G. M. Wuts, Wiley-Interscience (2007); and ‘Protecting Groups’, 3rd edition, P. J. Kocienski, Thieme (2005).

The compounds of formula (I) may be converted into a pharmaceutically acceptable salt thereof, preferably an acid addition salt such as a formate, hemi-formate, hydrochloride, hydrobromide, benzenesulfonate (besylate), saccharin (e.g. monosaccharin), trifluoroacetate, sulfate, nitrate, phosphate, acetate, fumarate, semi-fumarate, maleate, tartrate, lactate, citrate, pyruvate, succinate, valerate, propanoate, butanoate, malonate, oxalate, 1-hydroxy-2-naphthoate (xinafoate), methanesulfonate or p-toluenesulfonate salt. In one embodiment of the invention, the compounds of formula (I) are in the form of a hydrochloride, formate or fumarate salt.

A salt of a compound of formula (I) may also be formed between a protic acid functionality of a compound of formula (I) and a suitable cation. Suitable cations include, but are not limited to lithium, sodium, potassium, magnesium, calcium and ammonium. In one embodiment of the invention, the salt is a mono- or di-sodium salt or a mono- or di-potassium salt.

Compounds of formula (I) and their salts may be in the form of hydrates or solvates which form another embodiment of the present invention. Such solvates may be formed with common organic solvents including, but not limited to alcoholic solvents e.g. methanol, ethanol or isopropanol.

In one embodiment of the present invention, therapeutically inactive prodrugs are provided. Prodrugs are compounds which, when administered to a subject such as a human, are converted in whole or in part to a compound of formula (I). Generally, the prodrugs are pharmacologically inert chemical derivatives that can be converted in vivo to the active drug molecules to exert a therapeutic effect. Any of the compounds of formula (I) can be administered as a prodrug to increase the activity, bioavailability, or stability of the compound of formula (I) or to otherwise alter the properties of the compound of formula (I). Typical examples of prodrugs include compounds that have biologically labile protecting groups on a functional moiety of the active compound.

Prodrugs include but are not limited to compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

Where the compounds, salts, solvates and prodrugs of the present invention are capable of existing in stereoisomeric forms, it will be understood that the invention encompasses the use of all geometric and optical isomers (including atropisomers) and mixtures thereof. The use of tautomers and mixtures thereof also forms an embodiment of the present invention. The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, more typically less than 1%, and most typically less than 0.5% by weight. Enantiomerically pure isomers are particularly desired.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I. Therefore, the term “hydrogen”, for example, encompasses ¹H, ²H (D) and ³H (T). Similarly, carbon atoms are to be understood to include ¹¹C, ¹²C, ¹³C and ¹⁴C, nitrogen atoms are to be understood to include ¹³N, ¹⁴N and ¹⁵N, oxygen atoms are to be understood to include ¹⁵O, ¹⁶O, ¹⁷O and ¹⁸O, fluorine atoms are to be understood to include ¹⁸F and ¹⁹F, and iodine atoms are to be understood to include ¹²³I, ¹²⁴I, ¹²⁵I, ¹²⁷I and ¹³¹I.

In one embodiment, the compounds, salts, solvates and prodrugs of the present invention may be isotopically labelled. As used herein, an “isotopically labelled” compound is one in which the abundance of a particular nuclide at a particular atomic position within the molecule is increased above the level at which it occurs in nature. Any of the compounds, salts, solvates and prodrugs of the present invention can be isotopically labelled, for example, any of examples 1 to 83.

In one embodiment, the compounds, salts, solvates and prodrugs of the present invention may bear one or more radiolabels. Such radiolabels may be introduced by using radiolabel-containing reagents in the synthesis of the compounds, salts, solvates or prodrugs, or may be introduced by coupling the compounds, salts, solvates or prodrugs to chelating moieties capable of binding to a radioactive metal atom. Such radiolabelled versions of compounds, salts, solvates and prodrugs may be used, for example, in diagnostic imaging studies.

In one embodiment, the compounds, salts, solvates and prodrugs of the present invention may be tritiated, i.e., they contain one or more 3H (T) atoms. Any of the compounds, salts, solvates and prodrugs of the present invention can be tritiated, for example, any of examples 1 to 83.

The compounds, salts, solvates and prodrugs of the present invention may be amorphous or in a polymorphic form or a mixture of any of these, each of which is an embodiment of the present invention.

The compounds, salts, solvates and prodrugs of the present invention have activity as pharmaceuticals and may be used in treating or preventing a disease, disorder or condition associated with GPR55 activity.

Therefore, a fourth aspect of the present invention provides a compound of formula (I) or a pharmaceutically acceptable salt, solvate or prodrug thereof, according to the first aspect of the present invention, for use in therapy, in particular for use in treating or preventing a neurodegenerative disease, Parkinson's Disease, Alzheimer's Disease, depression, anxiety, an anxiety related disorder, epilepsy, or pain (including mechanical, inflammatory and neuropathic pain).

A fifth aspect of the present invention provides a use of a compound of formula (I) or a pharmaceutically acceptable salt, solvate or prodrug thereof, according to the first aspect of the present invention, for the manufacture of a medicament for treating or preventing a neurodegenerative disease, Parkinson's Disease, Alzheimer's Disease, depression, anxiety, an anxiety related disorder, epilepsy, or pain (including mechanical, inflammatory and neuropathic pain).

A sixth aspect of the present invention provides a method of treating or preventing a neurodegenerative disease, Parkinson's Disease, Alzheimer's Disease, depression, anxiety, an anxiety related disorder, epilepsy, or pain (including mechanical, inflammatory and neuropathic pain); the method comprising administering a therapeutically or prophylactically effective amount of a compound of formula (I) or a pharmaceutically acceptable salt, solvate or prodrug thereof, according to the first aspect of the present invention, to a patient in need thereof.

Unless stated otherwise, in any of the fourth, fifth or sixth aspects of the invention, the subject or patient may be any human or other animal. Typically, the subject or patient is a mammal, more typically a human or a domesticated mammal such as a cow, pig, lamb, sheep, goat, horse, cat, dog, rabbit, mouse etc. Most typically, the subject is a human.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disorder or condition in question. Persons at risk of developing a particular disorder or condition generally include those having a family history of the disorder or condition, or those who have been identified by genetic testing or screening to be particularly susceptible to developing the disorder or condition or those in the prodromal phase of a disorder.

The terms “treat”, “treatment” and “treating” include improvement of the conditions described herein. The terms “treat”, “treatment” and “treating” include all processes providing slowing, interrupting, arresting, controlling, or stopping of the state or progression of the conditions described herein, but does not necessarily indicate a total elimination of all symptoms or a cure of the condition. The terms “treat”, “treatment” and “treating” are intended to include therapeutic as well as prophylactic treatment of such conditions.

For the above-mentioned therapeutic uses the dosage administered will, of course, vary with the compound employed, the mode of administration, the treatment desired and the disorder indicated. For example, the daily dosage of a compound of the invention (that is, a compound of formula (I) or a pharmaceutically acceptable salt, solvate or prodrug thereof), if administered orally or parenterally, may be in the range from 0.01 micrograms per kilogram body weight (μg/kg) to 100 milligrams per kilogram body weight (mg/kg). Alternatively, if the compound is inhaled, then the daily dosage of the compound of the invention may be in the range from 0.05 micrograms per kilogram body weight (μg/kg) to 1 milligram per kilogram body weight (mg/kg). The desired dosage may be presented at an appropriate interval such as once every other day, once a day, twice a day, three times a day or four times a day.

The compounds of formula (I) and pharmaceutically acceptable salts, solvates and prodrugs thereof may be used on their own, but will generally be administered in the form of a pharmaceutical composition in which the active ingredient is in association with a pharmaceutically acceptable adjuvant, diluent or carrier.

Therefore, a seventh aspect of the present invention provides a pharmaceutical composition comprising a compound of formula (I) or a pharmaceutically acceptable salt, solvate or prodrug thereof, according to the first aspect of the present invention, in association with a pharmaceutically acceptable adjuvant, diluent or carrier, and optionally one or more other therapeutic agents.

The invention still further provides a process for the preparation of a pharmaceutical composition of the invention which comprises mixing a compound of formula (I) or a pharmaceutically acceptable salt, solvate or prodrug thereof, according to the first aspect of the present invention, with a pharmaceutically acceptable adjuvant, diluent or carrier.

Conventional procedures for the selection and preparation of suitable pharmaceutical formulations are described in, for example, “Pharmaceutics—The Science of Dosage Form Design”, M. E. Aulton, Churchill Livingstone, 1988.

Pharmaceutically acceptable adjuvants, diluents or carriers that may be used in the pharmaceutical compositions of the invention are those conventionally employed in the field of pharmaceutical formulation, and include, but are not limited to sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, dipotassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, rectally, nasally, buccally, vaginally, ocularly, topically or via an implanted reservoir. Oral administration is preferred. The pharmaceutical compositions of the invention may contain any conventional non-toxic pharmaceutically acceptable adjuvants, diluents or carriers. The term parenteral as used herein includes subcutaneous, intracutaneous, intradermal, intravenous, intramuscular, intra-articular, intrasynovial, intrasternal, intrathecal, intralesional, intracranial, intratracheal, intraperitoneal, intraarticular, and epidural injection or infusion techniques. The term topical as used herein includes transdermal, mucosal, sublingual and topical ocular administration.

The pharmaceutical compositions may be in the form of a sterile injectable preparation, for example, as a sterile injectable aqueous or oleaginous suspension. The suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable diluents and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to capsules, tablets, caplets, troches, lozenges, powders, granules, and aqueous suspensions, solutions and dispersions. These dosage forms are prepared according to techniques well-known in the art of pharmaceutical formulation. In the case of tablets for oral use, carriers which are commonly used include lactose, sodium and calcium carbonate, sodium and calcium phosphate, and corn starch. Lubricating agents, such as magnesium stearate, stearic acid or talc, are also typically added. If desired, the tablets may be coated with a material, such as glyceryl monostearate or glyceryl distearate, to delay absorption in the gastrointestinal tract. Tablets may also be effervescent and/or dissolving tablets. For oral administration in capsule form, useful diluents include lactose and dried corn starch. When aqueous suspensions are administered orally, the active ingredient may be combined with emulsifying and suspending agents. If desired, certain sweetening and/or flavouring and/or colouring agents and/or preservatives may be added to any oral dosage form.

The pharmaceutical compositions of the invention may also be administered in the form of suppositories for rectal administration. These compositions can be prepared by mixing the active ingredient with a suitable non-irritating excipient which is solid at room temperature but liquid at the rectal temperature and therefore will melt in the rectum to release the active ingredient. Such materials include, but are not limited to cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels, and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Depending on the mode of administration, the pharmaceutical composition will preferably comprise from 0.05 to 99% by weight, more preferably from 0.05 to 80% by weight, still more preferably from 0.1 to 70% by weight, and even more preferably from 0.1 to 50% by weight of active ingredient, all percentages by weight being based on total composition.

The compounds of the invention may also be administered in conjunction with other compounds used for the treatment of the above conditions.

The invention therefore further relates to combination therapies wherein a compound of the invention or a pharmaceutical composition or formulation comprising a compound of the invention is administered with another therapeutic agent or agents for the treatment of one or more of the conditions previously indicated. The compound of the invention or the pharmaceutical composition or formulation comprising the compound of the invention may be administered simultaneously with, separately from or sequentially to the one or more other therapeutic agents. The compound of the invention and the one or more other therapeutic agents may be comprised in the same pharmaceutical composition or formulation, or in separate pharmaceutical compositions or formulations, i.e. in the form of a kit. The other therapeutic agent may be L-DOPA.

Typically, the mode of administration selected is that most appropriate to the disorder, disease or condition to be treated or prevented. Where one or more further active agents are administered, the mode of administration may be the same as or different to the mode of administration of the compound or pharmaceutical composition of the invention.

Such combination products employ the compounds of this invention within the dosage range described herein and the other pharmaceutically active agent(s) within approved dosage ranges.

### BRIEF DESCRIPTION OF DRAWINGS

FIG. 1 illustrates a comparison of the firing frequency of medium spiny neurons using vehicle control (DMSO) relative to baseline.

FIG. 2 illustrates a comparison of the firing frequency of medium spiny neurons using the compound of Example 6 (10 μM) relative to baseline.

FIG. 3 illustrates a comparison of the firing frequency of medium spiny neurons using the compound of Example 31 (1 μM) relative to baseline.

### DEFINITIONS

An “alkyl” group may be linear (i.e., straight-chained) or branched. Examples of alkyl groups include methyl, ethyl, n-propyl, iso-propyl, n-butyl, iso-butyl, sec-butyl, tert-butyl, n-pentyl, 2-methyl-1-butyl, 3-methyl-1-butyl, 3-methyl-2-butyl, and 2,2-dimethyl-1-propyl groups. Unless stated otherwise, the term “alkyl” does not include “cycloalkyl”. Typically, an alkyl group is a C₁-C₁₂ alkyl group. More typically an alkyl group is a C₁-C₆ or C₁-C₅ alkyl group. An “alkylene” group is similarly defined as a divalent alkyl group.

An “alkenyl” group is an unsaturated alkyl group having one or more carbon-carbon double bonds. Examples of alkenyl groups include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups. Unless stated otherwise, the term “alkenyl” does not include “cycloalkenyl”. Typically, an alkenyl group is a C₂-C₁₂ alkenyl group. More typically an alkenyl group is a C₂-C₆ or C₂-C₅ alkenyl group. An “alkenylene” group is similarly defined as a divalent alkenyl group.

An “alkynyl” group is an unsaturated alkyl group having one or more carbon-carbon triple bonds. Examples of alkynyl groups include ethynyl, propargyl, but-1-ynyl and but-2-ynyl groups. Typically, an alkynyl group is a C₂-C₁₂ alkynyl group. More typically an alkynyl group is a C₂-C₆ or C₂-C₅ alkynyl group. An “alkynylene” group is similarly defined as a divalent alkynyl group.

An “alkoxy” group is an —O-alkyl group. Examples of alkoxy groups include methoxy, ethoxy, n-propoxy, iso-propoxy, n-butoxy, iso-butoxy, sec-butoxy, tert-butoxy, n-pentoxy groups. Typically, an alkoxy group is a C₁-C₁₂ alkoxy group. More typically an alkoxy group is a C₁-C₆ or C₁-C₅ alkoxy group.

A “cycloalkyl” group is a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl group may be monocyclic, bicyclic (e.g., bridged, fused or spiro), or polycyclic.

A “cycloalkenyl” group is a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl group may be monocyclic, bicyclic (e.g., bridged, fused or spiro), or polycyclic.

An “aryl” group is an aromatic hydrocarbyl ring. The term “aryl” includes monocyclic aromatic hydrocarbons (such as phenyl) and polycyclic fused-ring aromatic hydrocarbons (such as naphthyl, anthracenyl and phenanthrenyl). Unless stated otherwise, the term “aryl” does not include “heteroaryl”.

A “heterocyclic” group is a non-aromatic cyclic group which includes one or more carbon atoms and one or more (such as one, two, three or four) heteroatoms, e.g. N, O or S, in the ring structure. A heterocyclic group may be monocyclic, bicyclic (e.g. bridged, fused or spiro), or polycyclic. Typically, a heterocyclic group is a 4- to 14-membered heterocyclic group, which means it contains from 4 to 14 ring atoms. More typically, a heterocyclic group is a 4- to 10-membered heterocyclic group, which means it contains from 4 to 10 ring atoms. Heterocyclic groups include unsaturated heterocyclic groups (such as azetinyl, tetrahydropyridinyl, and 2-oxo-1H-pyridinyl) and saturated heterocyclic groups. Examples of saturated monocyclic heterocyclic groups are azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups. Examples of saturated bicyclic heterocyclic groups are quinuclidinyl, 8-azabicyclo[3.2.1]octanyl, 2-azaspiro[3.3]heptanyl, 6-azaspiro[2.5]octanyl and hexahydro-1H-pyrrolizinyl groups.

A “heteroaryl” group is an aromatic cyclic group which includes one or more carbon atoms and one or more (such as one, two, three or four) heteroatoms, e.g. N, O or S, in the ring structure. Typically, a heteroaryl group is a 5- to 14-membered heteroaryl group, which means it contains from 5 to 14 ring atoms. More typically, a heteroaryl group is a 5- to 10-membered heteroaryl group, which means it contains from 5 to 10 ring atoms. The term “heteroaryl” includes monocyclic aromatic heterocycles (such as pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyridazinyl, pyrimidinyl, pyrazinyl, triazinyl and tetrazinyl) and polycyclic fused-ring aromatic heterocycles (such as indolyl, benzofuranyl, benzothiophenyl, benzoxazolyl, benzisoxazolyl, benzothiazolyl, benzoisothiazolyl, benzimidazolyl, 1H-imidazo[4,5-b]pyridinyl, 1H-imidazo[4,5-c]pyridinyl, quinolinyl, isoquinolinyl, quinoxalinyl, quinazolinyl, phthalazinyl and cinnolinyl). Examples of heteroaryl groups include the following:

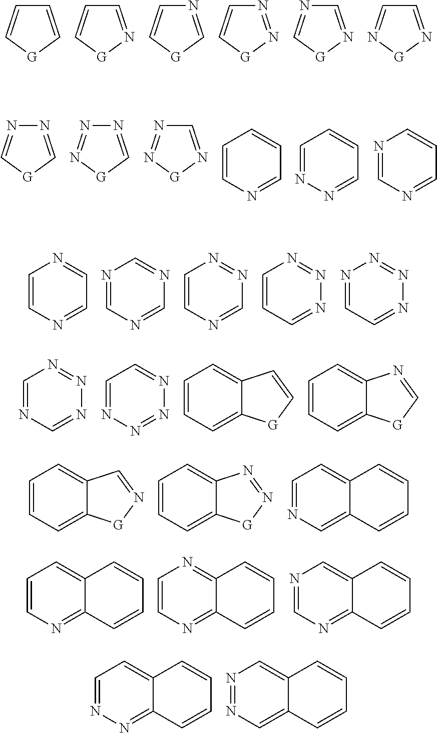

wherein G=0, S or NH.

For the purposes of the present specification, where a combination of moieties is referred to as one group, for example, arylalkyl, arylalkenyl, arylalkynyl, alkylaryl, alkenylaryl or alkynylaryl, the last-mentioned moiety contains the atom by which the group is attached to the rest of the molecule. An example of an arylalkyl group is benzyl.

The term “halo” includes fluoro, chloro, bromo and iodo. In one embodiment, halo is fluoro.

Unless stated otherwise, where a group is prefixed by the term “halo”, such as a “haloalkyl” or “halomethyl” group, it is to be understood that the group in question is substituted with one or more (such as one, two, three, four or five) halo groups independently selected from fluoro, chloro, bromo and iodo. Typically, the maximum number of halo substituents is limited only by the number of hydrogen atoms available for substitution on the corresponding group without the halo prefix. For example, a “halomethyl” group may contain one, two or three halo substituents. A “haloethyl” or “halophenyl” group may contain one, two, three, four or five halo substituents. Similarly, unless stated otherwise, where a group is prefixed by a specific halo group, it is to be understood that the group in question is substituted with one or more (such as one, two, three, four or five) of the specific halo groups. For example, the term “fluoromethyl” refers to a methyl group substituted with one, two or three fluoro groups, and the term “fluoroethyl” refers to an ethyl group substituted with one, two, three, four or five fluoro groups.

A “hydroxyalkyl” group is an alkyl group substituted with one or more (such as one, two or three) hydroxyl (—OH) groups. Typically, a hydroxyalkyl group has one or two hydroxyl substituents, more typically a hydroxyalkyl group has one hydroxyl substituent.

Unless stated otherwise, any reference to an element is to be considered a reference to all isotopes of that element. Thus, for example, unless stated otherwise, any reference to hydrogen is considered to encompass all isotopes of hydrogen including ¹H, ²H (D) and ³H (T). Therefore, for the avoidance of doubt, it is noted that, for example, the terms “alkyl” and “methyl” include, for example, trideuteriomethyl.

Unless stated otherwise, any reference to a compound or group is to be considered a reference to all tautomers of that compound or group.

When any chemical group or moiety is described as substituted, it will be appreciated that the number and nature of substituents will be selected so as to avoid sterically undesirable combinations.

For the avoidance of doubt the term “5-(2-((1-phenylethyl)amino)pyrimidin-5-yl)-1,3-dihydro-2H-benzo[d]imidazol-2-one” includes individual stereoisomers and any mixtures thereof (e.g. racemic mixtures).

For the avoidance of doubt the term “5-(benzo[d][1,3]dioxol-5-yl)-N-(1-(4-fluorophenyl)ethyl)pyridin-2-amine” includes individual stereoisomers and any mixtures thereof (e.g. racemic mixtures).

### EXAMPLES

The present invention will now be further explained by reference to the following illustrative examples, in which the starting materials and reagents used are available from commercial suppliers or prepared via literature procedures or procedures similar to the ones described in this application.

Nuclear magnetic resonance (NMR) spectra were recorded at 400 MHz at room temperature unless otherwise stated; the chemical shifts (δ) are reported in parts per million. Spectra were recorded using a Bruker™ 400 AVANCE instrument fitted with a 5 mm BBFO probe with instrument controlled by Bruker TopSpin 2.1 software, or a Bruker 400 AVANCE-III HD instrument fitted with a 5 mm BBO smart probe or a 5 mm BBFO probe with instrument controlled by Bruker TopSpin 3.5 software, or a Bruker 400 AVANCE-III instrument fitted with a 5 mm BBFO probe with instrument controlled by Bruker Topspin 3.0 software.

Reactions were monitored using one or more of the following:

Agilent 1290 infinity II UPLC coupled with 6130 quadrupole LCMS; chromatographic conditions: mobile phase A: 0.1% HCOOH in H₂O; mobile phase B: 0.1% HCOOH in CH₃CN; column: Acquity UPLC BEH C18 (50 mm×2.1 mm, 1.7 m); column T: 40° C.; Sample T: RT; detection at 220 nm; flow rate: 0.7 mL/min; analysis time: 4.2 min; mass range: 100 to 1500.

Dionex Ultima 3000 UHPLC Coupled with Thermo Scientific LCQ Fleet Ion Trap. Chromatographic conditions: mobile phase A: 10 mM ammonium formate in H₂O:CH₃CN (95:5); mobile phase B: 10 mM ammonium formate in H₂O:CH₃CN (5:95); column: XBridge BEH C18 (50 mm×3.0 mm, 2.5 μm); column T: 40° C.; sample T: RT; detection at 220 nm; flow rate: 0.7 mL/min; analysis time: 4.2 min; mass range: 100 to 1500.

ACQUITY UPLC H-Class with single quadrupole LCMS; chromatographic conditions: mobile phase A: 0.1% HCOOH in H₂O; mobile phase B: 0.1% HCOOH in CH₃CN; column: Acquity UPLC BEH C18 (50 mm×2.1 mm, 1.7 m); column T: 40° C.; sample T: RT; detection at 220 nm; flow rate: 0.7 mL/min; analysis time: 4.2 min; mass range: 100 to 1500.

Purity was assessed using one or more of the following:

Ultra Performance Liquid Chromatography (UPLC) with UV (photodiode array) detection over a wide range of wavelengths, normally 220-450 nm, using a H₂O s Acquity™ UPLC system equipped with Acquity UPLC BEH, HSS or HSS T3 C18 columns (2.1 mm id×50 mm) operated at 50 or 60° C. Mobile phases typically consisted of CH₃CN mixed with H₂O containing either 0.1% formic acid, 0.1% TFA or 0.025% ammonia. Mass spectra were recorded with a H₂O s SQD single quadrupole mass spectrometer using atmospheric pressure ionisation.

UPLC with UV (photodiode array) detection over a wide range of wavelengths, normally 220-450 nm, using Shimadzu™ Nexera X2 UPLC controlled by Lab Solution software equipped with Acquity UPLC BEH, HSS or HSS T3 C18 columns (2.1 mm id×50 mm), operated at 50° C. Mobile phases typically consisted of CH₃CN mixed with H₂O containing either 0.1% formic acid, 0.1% TFA or 0.025% ammonia. Mass spectra were recorded with a Shimadzu single quadrupole mass spectrometer using DUIS ionisation.

Unless stated otherwise, compounds were purified using Grace purifier, Buchi Reveleris X2 flash purification system or Biotage using normal phase chromatography on silica, using Reveleris SRC flash cartridges, Interchim™ PuriFlash cartridges or Kinesis™ Telos silica cartridges, or on basic silica using Biotage KP-NH cartridges, or by reverse phase chromatographic methods using Reveleris RP flash cartridges or by Biotage Isolute SCX-2 or Phenomenex™ Strata ABW using catch-release cartridges, or by preparative high performance liquid chromatography (HPLC).

Preparative HPLC was performed using Agilent Technologies, 1100 Series system or a H₂O autopurification LC/MS system typically using H₂O (19 mm id×250 mm C18 columns such as XBridge or SunFire 5 μm) at RT. Mobile phases typically consisted of CH₃CN mixed with H₂O containing either 0.1% formic acid or 0.1% ammonia, unless otherwise stated.

Unless stated otherwise, super Critical Fluid Chromatography (SFC) chiral separations were performed on a H₂O s SFC investigator system, using a flow rate of 60-120 g/min, T=RT to 40° C. and a pressure of 100 bar. Mobile phases typically consisted of supercritical CO₂ and a polar solvent such as CH₃CN, MeOH, EtOH or isopropanol, respectively. Column type and eluent are detailed for individual examples. Columns: Chiralcel OJ-H (250 mm×21 mm, 5 μm), Chiralpak-IG (250 mm×30 mm, 5 μm), Chiralpak-IE (250 mm×30 mm, 5 μm), Chiralpak-AD-H (250 mm×30 mm, 5 μm), Chiralcel OX-H (250 mm×21 mm, 5 μm), YMC SC (250 mm×30 mm, 5 μm), Chiralpak AS-H (250 mm×30 mm, 5 μm), R,R Whelk-01 (250 mm×30 mm, 5 μm), Chromega chiral CCO (250 mm×30 mm, 5 μm), Chromega Chiral CCA (250 mm×30 mm, 5 μm), Chiralpak IA (250 mm×30 mm, 5 μm), Lux Cellulose-02 (250 mm×30 mm, 5 μm), Chiralpak OD-H (250 mm×30 mm, 5 μm); UV detection at 200-400 nm; sample diluent: CH₃CN, MeOH; injection: 0.1 mL to 5 mL; isocratic ratio: 5-50% of mobile phase.

‘Room temperature’, as used in the present specification, means a temperature in the range from about 18° C. to about 25° C.

For the purposes of the present invention, for all of the experimental details described below, where there are reaction conditions described, such as reagents, solvents and temperatures, above and/or below an arrow in a graphical representation, it is to be understood that these reaction conditions, in particular solvents and temperatures, are not essential to the reaction being carried out and may be varied.

### Abbreviations

aq. aqueous
DAST N,N-diethylaminosulfur trifluoride
DIPEA N,N-diisopropylethylamine
DMF dimethylformamide
EtOAc ethyl acetate
h hour
HPLC high-performance liquid chromatography
MeOH methanol
min minute
NaH sodium hydride
NMP N-methyl-2-pyrrolidone
Pd₂(dba)₃ tris(dibenzylideneacetone)dipalladium(0)
RT room temperature
sat. saturated
TBAF tetra-n-butylammonium fluoride
TEA triethylamine
TFA trifluoroacetic acid
THF tetrahydrofuran

### 1. Synthetic Intermediates

Commercial intermediates were used in preparation of the examples except for the following intermediates which were prepared using standard procedures as outlined below.

### Intermediate 1: N-(1-(4-fluorophenyl)ethyl)-5-(4,4,5,5-tetramethyl-1,3,2-dioxaborolan-2-yl)pyridin-2-amine

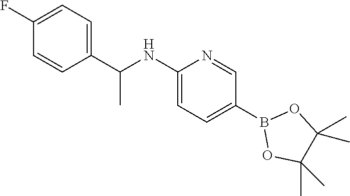 [SMILES: CC(Nc1ccc(B2OC(C)(C)C(C)(C)O2)cn1)c1ccc(F)cc1]

Step 1: A solution of 5-bromo-2-chloropyridine (5.0 g, 26 mmol), 1-(4-fluorophenyl)ethan-1-amine (4.34 g, 31.2 mmol) and Cs₂CO₃ (16.94 g, 52 mmol) in toluene (50 mL) was purged with N₂ for 10 min; rac-BINAP (1.62 g, 2.6 mmol) and Pd₂(dba)₃ (1.19 g, 1-3 mmol) were added under N₂. The tube was sealed and the mixture stirred at 110° C. for 2 h, diluted with EtOAc (100 mL) and filtered through Celite®. The filtrate was concentrated and the residue purified by reverse phase column chromatography (C18, 40 g snap, 0-80% MeOH in 0.1% ammonium bicarbonate in H₂O) to give 5-bromo-N-(1-(4-fluorophenyl)ethyl)pyridin-2-amine (1.05 g, 13%) as a yellow solid. ¹H NMR (400 MHz, DMSO-d₆): δ 7.95 (d, J=2.40 Hz, 1H), 7.50-7.46 (m, 1H), 7.39-7.34 (m, 2H), 7.25 (d, J=7.60 Hz, 1H), 7.13-7.07 (m, 2H), 6.48 (d, J=9.20 Hz, 1H), 4.97-4.93 (m, 1H), 1.40 (d, J=7.20 Hz, 3H). MS m/z: 295.04.

Step 2: A solution of 5-bromo-N-(1-(4-fluorophenyl)ethyl)pyridin-2-amine (1.0 g, 3.4 mmol), bis(pinacolato)diboron (1.77 g, 6.80 mmol) and KOAc (0.83 g, 8.55 mmol) in dioxane (10 mL) was purged with N₂ for 10 min, treated with PdCl₂(dppf) (0.25 g, 0.34 mmol) and stirred at 100° C. for 16 h. The mixture was diluted with EtOAc (100 mL) and filtered through Celite®. The filtrate was concentrated under reduced pressure to afford crude N-(1-(4-fluorophenyl)ethyl)-5-(4,4,5,5-tetramethyl-1,3,2-dioxaborolan-2-yl)pyridin-2-amine (3.0 g) as black gum, which was used for the next step without further purification. MS m/z: 261.04 (boronic acid).

### Intermediate 2: 6′-chloro-5-methyl-3,3′-bipyridine

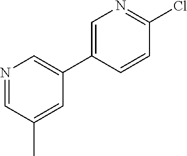

Step 1: A solution of 5-bromo-2-chloropyridine (2.00 g, 10.4 mmol), bis(pinacolato)diboron 3.16 g, 12.5 mmol) and KOAc (2.56 g, 25.98 mmol) in dioxane (20 mL) was purged with N₂ for 10 min, treated with PdCl₂(dppf) (0.76 g, 0.104 mmol) and stirred at 100° C. for 2 h. The mixture was diluted with EtOAc (100 mL) and filtered through Celite®. The filtrate was concentrated under reduced pressure to afford a brown gum, which was purified by reverse phase column chromatography (0-50% MeOH in 0.1% ammonium bicarbonate in H₂O) to give 2-chloro-5-(4,4,5,5-tetramethyl-1,3,2-dioxaborolan-2-yl)pyridine as a pink semi-solid (1.47 g, 59%). MS m/z: 157.99 (boronic acid).

Step 2: A solution of 3-bromo-5-methylpyridine (0.6 g, 3.49 mmol), 2-chloro-5-(4,4,5,5-tetramethyl-1,3,2-dioxaborolan-2-yl)pyridine 1.25 g, 5.23 mmol) and K₂CO₃ in dioxane (5.4 mL) and H₂O (0.6 mL) under N₂ was treated with PdCl₂(dppf) (0.26 g, 0.35 mmol), stirred at 100° C. for 16 h, diluted with EtOAc (100 mL) and filtered through Celite®. The filtrate was concentrated under reduced pressure. Purification of the residue by flash column chromatography (0-50% EtOAc in petroleum ether) gave 6′-chloro-5-methyl-3,3′-bipyridine as a white solid (0.36 g, 50%). ¹H NMR (400 MHz, DMSO-d₆): δ 8.80-8.76 (m, 2H), 8.49 (d, J=1.20 Hz, 1H), 8.24 (dd, J=2.40, 8.40 Hz, 1H), 8.01 (d, J=0.80 Hz, 1H), 7.65 (dd, J=0.80, 8.40 Hz, 1H), 2.38 (s, 3H). MS m/z: 205.07.

### Intermediate 3: (R)-N-(1-(4-fluorophenyl)ethyl)-5-(4,4,5,5-tetramethyl-1,3,2-dioxaborolan-2-yl)pyridin-2-amine

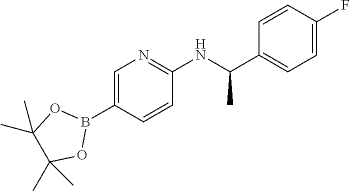 [SMILES: C[C@@H](Nc1ccc(B2OC(C)(C)C(C)(C)O2)cn1)c1ccc(F)cc1]

Step 1: A solution of 5-bromo-2-chloropyridine (4.50 g, 23.4 mmol), (R)-1-(4-fluorophenyl)ethan-1-amine (3.90 g, 28.1 mmol) and DIPEA (6.20 mL, 35.1 mmol) in N-methyl-2-pyrrolidone (20 mL) was stirred at 170° C. for 72 h in a sealed tube. The mixture was diluted with EtOAc (100 mL) and filtered through Celite®. The filtrate was concentrated under reduced pressure and the residue purified by reverse phase column chromatography (0-80% MeOH in 0.1% ammonium bicarbonate in H₂O) to give (R)-5-bromo-N-(1-(4-fluorophenyl)ethyl)pyridin-2-amine as pale brown liquid (2.0 g, 29%). ¹H NMR (400 MHz, DMSO-d₆): δ 7.95 (d, J=2.40 Hz, 1H), 7.50-7.46 (m, 1H), 7.39-7.34 (m, 2H), 7.25 (d, J=7.60 Hz, 1H), 7.13-7.07 (m, 2H), 6.48 (d, J=9.20 Hz, 1H), 4.97-4.93 (m, 1H), 1.40 (d, J=7.20 Hz, 3H). MS m/z: 295.35.

Step 2: The desired product was obtained by following the procedure employed for Intermediate 1 (step 2) using (R)-5-bromo-N-(1-(4-fluorophenyl)ethyl) pyridin-2-amine (1.0 g, 3.4 mmol). (R)-N-(1-(4-fluorophenyl)ethyl)-5-(4,4,5,5-tetramethyl-1,3,2-dioxaborolan-2-yl)pyridin-2-amine was obtained as a black gum (2.7 g), which was used for the next step without further purification. MS m/z: 261.11 (boronic acid).

### Intermediate 4: 3-bromo-5-(methylsulfinyl)pyridine

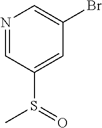

A solution of 3-bromo-5-(methylthio)pyridine (200 mg, 1 mmol) in AcOH (3 mL) was treated with NaIO₄ (320 mg, 1.5 mmol in H₂O, 0.5 mL) and stirred at RT for 12 h. The mixture was quenched with aq. NaHCO₃ and extracted with EtOAc. The combined organic phases were dried over Na₂SO₄ and evaporated under reduced pressure to obtain crude product. Purification by flash chromatography (20-30% EtOAc in petroleum ether) gave 3-bromo-5-(methylsulfinyl)pyridine as colourless liquid (160 mg, 70%). MS m/z: 220.11.

### Intermediate 5: 3-bromo-5-(ethylsulfinyl)pyridine

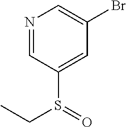

A solution of 3-bromo-5-(ethylthio)pyridine (1.0 g, 4.6 mmol) in AcOH (1.2 mL) was treated with NaIO₄ (1.7 g, 8.2 mmol in H₂O, 2 mL) and stirred at RT for 12 h. The mixture was quenched with aq. NaHCO₃ and extracted with EtOAc. The combined organic layers were dried over Na₂SO₄ and evaporated under reduced pressure to obtain crude product. Purification by flash chromatography (20-30% EtOAc in petroleum ether) gave 3-bromo-5-(ethylsulfinyl)pyridine as a colourless liquid (200 mg, 190%). ¹H NMR (400 MHz, CDCl₃): δ 8.79 (s, 1H), 8.62 (s, 1H), 8.19 (s, 1H), 3.04-3.00 (m, 1H), 2.98-81 (m, 1H), 1.26 (t, J=2.40 Hz, 3H). MS m/z: 236.14.

### Intermediate 6: 3-bromo-5-(ethylsulfonyl)pyridine

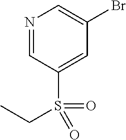

A solution of 3-bromo-5-(ethylthio)pyridine (1 g, 4.6 mmol) in AcOH (1.2 mL) was treated with NaIO₄ (1.7 g, 8.2 mmol) in H₂O (2 mL) and stirred at RT for 12 h. The mixture was quenched with aq. NaHCO₃ and extracted with EtOAc. The combined organic layers were dried over Na₂SO₄ and evaporated under reduced pressure to obtain crude product. Purification by flash chromatography (20-30% EtOAc in petroleum ether) gave bromo-5-(ethylsulfonyl)pyridine as a colourless liquid (600 mg, 60%). ¹H NMR (400 MHz, CDCl₃): δ 9.02 (s, 1H), 8.95 (s, 1H), 8.33 (s, 1H), 3.19 (q, J=7.60 Hz, 2H), 1.35 (t, J=7.20 Hz, 3H). MS m/z: 250.12.

### Intermediate 7: 1-(5-bromopyridin-3-yl)ethan-1-ol

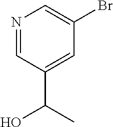

A mixture of 1-(5-bromopyridin-3-yl)ethan-1-one (500 mg, 2.5 mmol) in MeOH (5 mL) was slowly treated with sodium borohydride (190 mg, 5.0 mmol) at 0° C. in portions and stirred at RT for 6 h. The mixture was concentrated and extracted with EtOAc. The combined organic layers were dried over Na₂SO₄ and evaporated under reduced pressure to obtain crude product. Purification by flash chromatography (30% EtOAc in petroleum ether) gave 1-(5-bromopyridin-3-yl)ethan-1-ol as a yellow liquid (500 mg, 99%). ¹H NMR (400 MHz, DMSO-d₆): δ 8.57-8.54 (m, 2H), 7.98-7.97 (m, 1H), 4.82-4.65 (m, 1H), 1.36 (d, J=6.40 Hz, 3H). MS m/z: 202.

### Intermediate 8: (R)-(5-((1-(4-fluorophenyl)ethyl)amino)pyrazin-2-yl)boronic acid

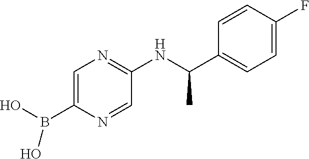 [SMILES: C[C@@H](Nc1cnc(B(O)O)cn1)c1ccc(F)cc1]

Step 1: (R)-5-chloro-N-(1-(4-fluorophenyl)ethyl)pyrazin-2-amine was obtained by following the procedure employed for Intermediate 3 (step 1) using 2,5-dichloropyrazine (1.50 g, 10.1 mmol), (R)-1-(4-fluorophenyl)ethan-1-amine (1.4 g, 10.1 mmol) and DIPEA (5.3 mL, 30.3 mmol), resulting in (R)-5-chloro-N-(1-(4-fluorophenyl)ethyl)pyrazin-2-amine as a pale brown solid (1.2 g, 47%). ¹H NMR (400 MHz, CDCl₃): δ 7.98 (s, 1H), 7.51 (s, 1H), 7.32-7.26 (m, 2H), 7.04-6.99 (m, 2H), 4.95 (d, J=5.20 Hz, 1H), 4.85-4.82 (m, 1H), 2.00 (d, J=6.80 Hz, 3H). MS m/z: 252.07.

Step 2: A solution of (R)-5-chloro-N-(1-(4-fluorophenyl)ethyl)pyrazin-2-amine (0.20 g, 0.80 mmol), bis(pinacolato)diboron (0.224 g, 0-95 mmol) and KOAc (0.194 g 1.99 mmol) in dioxane (5 mL) under N₂ was treated with tricyclohexylphosphine (0.044 g, 0.16 mmol) and Pd(OAc)₂ (0.018 g, 0.08 mmol). The mixture was stirred at 110° C. for 2 h, cooled to RT, diluted with EtOAc (50 mL) and filtered through Celite®. The filtrate was concentrated to afford crude (R)-(5-((1-(4-fluorophenyl)ethyl)amino)pyrazin-2-yl)boronic acid as a black gum (0.90 g), which was used for the next step without further purification. MS m/z: 262.15.

### Intermediate 9: (R)-N-(1-(2-fluorophenyl)ethyl)-5-(4,4,5,5-tetramethyl-1,3,2-dioxaborolan-2-yl)pyridin-2-amine

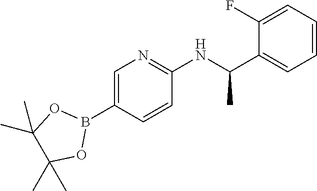 [SMILES: C[C@@H](Nc1ccc(B2OC(C)(C)C(C)(C)O2)cn1)c1ccccc1F]

Step 1: (R)-5-bromo-N-(1-(2-fluorophenyl)ethyl)pyridin-2-amine was prepared following the procedure employed for Intermediate 3 (step 1) using 5-bromo-2-chloropyridine (0.60 g, 3.12 mmol), (R)-1-(2-fluorophenyl)ethan-1-amine (0.52 g, 3.74 mmol) and DIPEA (1.1 mL, 6.24 mmol). Purification of the crude material by column chromatography gave the title compound as a pale brown solid (0.15 g, 16%). ¹HNMR (400 MHz, CDCl₃): δ 8.09 (d, J=2.00 Hz, 1H), 7.41-7.38 (m, 1H), 7.34-7.32 (m, 1H), 7.22-7.20 (m, 1H), 7.09-7.01 (m, 2H), 6.14 (d, J=8.80 Hz, 1H), 5.01-4.97 (m, 2H), 1.55 (d, J=3.20 Hz, 3H). MS m/z: 295.13.

Step 2: (R)-N-(1-(2-fluorophenyl)ethyl)-5-(4,4,5,5-tetramethyl-1,3,2-dioxaborolan-2-yl)pyridin-2-amine was prepared following the procedure employed for Intermediate 1 (step 2) using (R)-5-bromo-N-(1-(2-fluorophenyl)ethyl)pyridin-2-amine (0.15 g, 0.51 mmol). The crude title compound, a black gum (0.30 g), was used for the next step without further purification. MS m/z: 261.28 (boronic acid).

### Intermediate 10: 6′-chloro-5-ethyl-3,3′-bipyridine

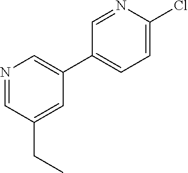

Title compound was prepared following the procedure employed for Intermediate 2 (step 2) using 3-bromo-5-ethylpyridine (2.0 g, 10.8 mmol) and (6-chloropyridin-3-yl)boronic acid (1.86 g, 11.9 mmol). Purification of the crude material by column chromatography gave 6′-chloro-5-ethyl-3,3′-bipyridine (1.1 g, 46%) as a colourless solid. ¹H NMR (400 MHz, DMSO-d₆): δ 8.63-8.60 (m, 2H), 8.53 (d, J=2.00 Hz, 1H), 7.85 (dd, J=2.40, 8.20 Hz, 1H), 7.67-7.65 (m, 1H), 7.45 (dd, J=0.80, 8.40 Hz, 1H), 2.75 (q, J=7.60 Hz, 2H), 2.00 (t, J=7.60 Hz, 3H). MS m/z: 219.12.

### Intermediate 11: (R)-5′-bromo-N-(1-(4-fluorophenyl)ethyl)-[3,3′-bipyridin]-6-amine

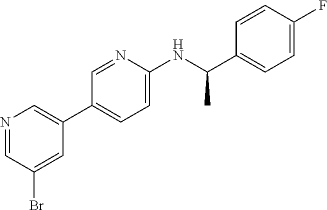 [SMILES: C[C@@H](Nc1ccc(-c2cncc(Br)c2)cn1)c1ccc(F)cc1]

Title compound was prepared following the procedure employed for Intermediate 2 (step 2) using 3,5-dibromopyridine (0.346 g, 1.46 mmol) and (R)-N-(1-(4-fluorophenyl)ethyl)-5-(4,4,5,5-tetramethyl-1,3,2-dioxaborolan-2-yl)pyridin-2-amine (Intermediate 3) (0.50 g, 1.46 mmol). Purification of the crude product by column chromatography gave (R)-5′-bromo-N-(1-(4-fluorophenyl)ethyl)-[3,3′-bipyridin]-6-amine as a brown gummy solid (150 mg, 27%). ¹H NMR (400 MHz, DMSO-d₆): δ 8.79 (s, 1H), 8.55 (s, 1H), 8.36 (s, 1H), 8.23 (s, 1H), 7.81-7.74 (m, 1H), 7.42-7.36 (m, 3H), 2.00 (t, J=8.80 Hz, 2H), 6.59 (d, J=8.80 Hz, 1H), 5.08-5.07 (m, 1H), 1.44 (d, J=6.80 Hz, 3H). MS m/z: 372.12.

### Intermediate 12: (R)-N-(1-(4-fluorophenyl)ethyl)-5′-((trimethylsilyl)ethynyl)-[3,3′-bipyridin]-6-amine

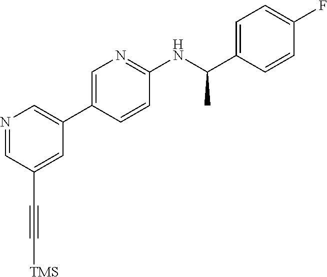 [SMILES: C[C@@H](Nc1ccc(-c2cncc(C#C[Si](C)(C)C)c2)cn1)c1ccc(F)cc1]

To a stirred solution of (R)-5′-bromo-N-(1-(4-fluorophenyl)ethyl)-[3,3′-bipyridin]-6-amine (Intermediate 11) (800 mg, 2.10 mmol) in toluene (20 mL) was added diethylamine (1.1 mL, 10.5 mmol), CuI (40 mg, 0.21 mmol), PdCl₂(PPh₃)₂ (150 mg, 0.21 mmol) and trimethylsilylacetylene (1.7 mL, 10.5 mmol). The mixture was heated at 100° C. for 16 h, cooled to RT and filtered through Celite®. The filtrate was concentrated and the residue purified by flash chromatography (20% EtOAc in petroleum ether) to give (R)-N-(1-(4-fluorophenyl)ethyl)-5′-((trimethylsilyl)ethynyl)-[3,3′-bipyridin]-6-amine as a brown sticky solid (600 mg, 71%). MS m/z: 390.26.

### Intermediate 13: 3-ethyl-5-(4,4,5,5-tetramethyl-1,3,2-dioxaborolan-2-yl)pyridine

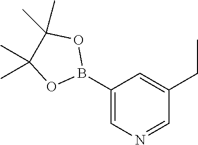 [SMILES: CCc1cncc(B2OC(C)(C)C(C)(C)O2)c1]

The title compound was prepared following the procedure employed for Intermediate 2 (step 1) using 3-bromo-5-ethylpyridine (0.25 g, 1.3 mmol), yielding crude 3-ethyl-5-(4,4,5,5-tetramethyl-1,3,2-dioxaborolan-2-yl)pyridine (0.2 g, 64%) as a brown sticky solid, which was used for the next step without further purification. MS m/z: 152.11 (boronic acid).

### Intermediate 14: (R)-5-bromo-N-(2,3-dihydro-1H-inden-1-yl)pyridin-2-amine

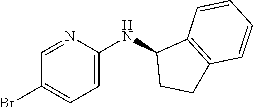 [SMILES: Brc1ccc(N[C@@H]2CCc3ccccc32)nc1]

The title compound was prepared following the procedure employed for Intermediate 3 (step 1) using 5-bromo-2-chloropyridine (0.25 g, 1.29 mmol) and (R)-2,3-dihydro-1H-inden-1-amine (0.259 g, 1.94 mmol) in N-methyl-2-pyrrolidone (6 mL). Purification of the crude material by column chromatography (Davisil silica, 30-50% EtOAc in petroleum ether) gave (R)-5-bromo-N-(2,3-dihydro-1H-inden-1-yl)pyridin-2-amine (0.10 g, 26%). MS m/z: 289.11.

### Intermediate 15: (R)-N-(1-(4-fluorophenyl)ethyl)-6-methyl-5-(4,4,5,5-tetramethyl-1,3,2-dioxaborolan-2-yl)pyridin-2-amine

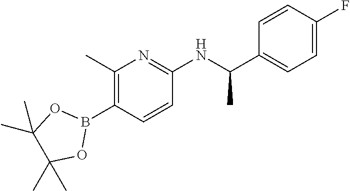 [SMILES: Cc1nc(N[C@H](C)c2ccc(F)cc2)ccc1B1OC(C)(C)C(C)(C)O1]

Step 1: (R)-5-bromo-N-(1-(4-fluorophenyl)ethyl)-6-methylpyridin-2-amine was prepared following the procedure employed for Intermediate 3 (step 1) using 3-bromo-6-chloro-2-methylpyridine (1.0 g, 4.8 mmol), (R)-1-(4-fluorophenyl)ethan-1-amine (0.80 ml, 5.76 mmol) and DIPEA (1.2 ml, 9.6 mmol). Purification of the crude material by column chromatography (Davisil silica, eluted from 30-50% EtOAc in petroleum ether) afforded (R)-5-bromo-N-(1-(4-fluorophenyl)ethyl)-6-methylpyridin-2-amine (0.21 g, 15%) as an off-white solid. MS m/z: 310.09.

Step 2: (R)-N-(1-(4-fluorophenyl)ethyl)-6-methyl-5-(4,4,5,5-tetramethyl-1,3,2-dioxaborolan-2-yl)pyridin-2-amine was prepared following the procedure employed for Intermediate 1 (step 2) using (R)-5-bromo-N-(1-(4-fluorophenyl)ethyl)-6-methylpyridin-2-amine (0.12 g, 0.38 mmol). The crude title compound (0.23 g) was obtained as pale yellow thick liquid and used for the next step without further purification. MS m/z: 357.18.

### Intermediate 16: (5-bromopyridin-3-yl)(imino)(methy)-λ⁶-sulfanone

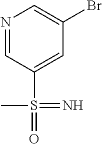

A solution of 3-bromo-5-(methylthio)pyridine (1.5 g, 7.3 mmol) in MeOH (10 mL) was treated with iodobenzene diacetate (5.8 g, 18.3 mmol) and ammonium carbamate (1.40 g, 18.3 mmol) and stirred at RT for 3 h. Excess solvent was removed under vacuo to give the crude compound, which was purified by reverse phase chromatography (30% H₂O:MeOH) to furnish (5-bromopyridin-3-yl)(imino)(methyl)-λ6-sulfanone as an off-white solid (1.0 g, 58%). ¹H NMR (400 MHz, DMSO-d₆): δ 9.03 (d, J=2.00 Hz, 1H), 8.98 (d, J=2.40 Hz, 1H), 8.48-8.47 (m, 1H), 4.67 (s, 1H), 3.32 (s, 3H). MS m/z: 234.98.

### Intermediate 17: (R)-6-chloro-N-(1-(4-chlorophenyl)ethyl)-1,2,4-triazin-3-amine

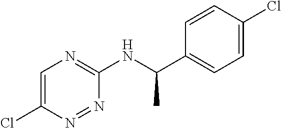 [SMILES: C[C@@H](Nc1ncc(Cl)nn1)c1ccc(Cl)cc1]

Step 1: A solution of 1,2,4-triazin-3-amine (500 mg, 5.2 mmol) in CH₃CN (10 mL) was treated with m-CPBA (1.17 g, 6.76 mmol) and stirred for 6 h at 70° C. The solvent was evaporated and the crude material triturated with Et₂O (5×100 mL), filtered and reduced under vacuum to give 3-amino-1,2,4-triazine 2-oxide as off-white solid (500 mg, 89%), which was used without further purification. MS m/z: 112.96 (M+H).

Step 2: To a stirred slurry of 3-amino-1,2,4-triazine 2-oxide (500 mg, 4.46 mmol) in HBr in H₂O (20 mL) was added NaNO₂ (1.5 g, 22.32 mmol, in 2 mL H₂O) slowly at 0° C. and the mixture was stirred for 1 h at RT. The mixture was concentrated and the residue dissolved in DCM, washed with aq. NaHCO₃ solution (5 mL), dried over Na₂SO₄ and concentrated under reduced pressure to give 3-bromo-1,2,4-triazine 2-oxide as a brown solid (300 mg, 38%). ¹H NMR (400 MHz, DMSO-d₆): δ 8.82 (s, 1H), 7.88 (s, 1H). MS m/z: 175.94 (M+H).

Step 3: To a stirred solution of 3-bromo-1,2,4-triazine 2-oxide (300 mg, 1.7 mmol) and (R)-1-(4-chlorophenyl)ethan-1-amine (277 mg, 1.78 mmol) in dioxane (10 mL) was added K₂CO₃ (704 mg, 5.1 mmol) and the reaction mixture was stirred for 1 h at 100° C. The solvent was evaporated under reduced pressure to get a residue which was triturated with Et₂O and decanted off to obtain (R)-3-((1-(4-chlorophenyl)ethyl)amino)-1,2,4-triazine 2-oxide as a thick liquid (600 mg, crude), which was used without further purification. MS m/z: 250.99.

Step 4: HCl gas was purged to a stirred solution of (R)-3-((1-(4-chlorophenyl)ethyl)amino)-1,2,4-triazine 2-oxide (600 mg, 2.4 mmol) in CHCl₃ for 3 h at RT. The mixture was stirred for 16 h at RT, treated with Na₂CO₃ to elevate the pH to ˜8 and the resulting mixture was stirred for 15 min. The mixture was filtered and the cake washed with CHCl₃. The filtrate was concentrated under reduced pressure to give (R)-6-chloro-N-(1-(4-chlorophenyl)ethyl)-1,2,4-triazin-3-amine (250 mg, 39%). ¹H NMR (400 MHz, DMSO-d₆): δ 8.58 (br s, 1H), 8.40 (s, 1H), 7.43-7.40 (m, 2H), 7.13 (t, J=9.2 Hz, 2H), 5.07 (br s, 1H), 1.17 (d, J=7.2 Hz, 3H). MS m/z: 268.96.

### Intermediate 18: 5-bromo-4-fluoro-N-(1-(4-fluorophenyl)ethyl)pyridin-2-amine

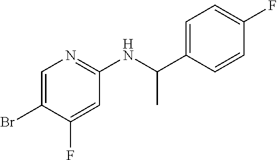 [SMILES: CC(Nc1cc(F)c(Br)cn1)c1ccc(F)cc1]

A solution of 5-bromo-4-fluoropyridin-2-amine (0.25 g, 1.3 mmol)) in DMF (3 mL) was treated with NaH (0.09 g, 3.9 mmol) at 0° C., stirred for 15 min, treated with 1-(1-bromoethyl)-4-fluorobenzene (0.319, 1.56 mmol) and stirred at RT for 16 h. The mixture was quenched with MeOH (5 mL) at ca. 4° C. and evaporated under reduced pressure. The residue was diluted with H₂O and extracted with CH₂Cl₂ (3×25 mL). The combined organic layers were washed with brine (2×15 mL), dried over anhydrous Na₂SO₄ and concentrated. Purification of the crude material by column chromatography (20-25% EtOAc in petroleum ether) gave 5-bromo-4-fluoro-N-(1-(4-fluorophenyl)ethyl)pyridin-2-amine (0.028 g, 68%) as a sticky solid. ¹H NMR (400 MHz, DMSO-d₆): δ 8.07 (d, J=10.4 Hz, 1H), 7.53 (d, J=8.0 Hz, 1H), 7.40-7.36 (m, 2H), 7.12 (t, J=8.8 Hz, 2H), 6.42 (d, J=11.6 Hz, 1H), 5.00-4.95 (m, 1H), 1.41 (d, J=7.20 Hz, 3H). MS m/z: 313.20.

### Intermediate 19: (5-bromopyridin-3-yl)(methyl)(methylimino)-λ⁶-sulfanone

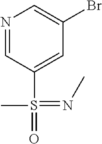

A solution of (5-bromopyridin-3-yl)(imino)(methyl)-λ⁶-sulfanone (500 mg, 2.10 mmol) in DMF (10 mL) was treated with NaH (55 mg, 2.31 mmol) at 0° C., stirred for 15 min, slowly treated with methyl iodide (238 mg, 1.68 mmol) and stirred at RT for 3 h. The mixture was quenched with ice-cold H₂O and extracted with EtOAc. The organic layers were concentrated and the residue purified by reverse phase chromatography (30% H₂O:MeOH) to give (5-bromopyridin-3-yl)(methyl)(methylimino)-λ6-sulfanone as an off-white solid (370 mg, 71%). ¹H NMR (400 MHz, DMSO-d₆): δ 9.01 (d, J=2.0 Hz, 1H), 8.92 (d, J=2.0 Hz, 1H), 8.40-8.39 (m, 1H), 3.30 (s, 3H), 2.50 (s, 3H). MS m/z: 248.99.

### Intermediate 20: 1-(5-bromopyridin-3-yl)-2,2,2-trifluoroethan-1-ol

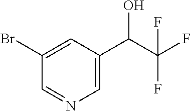 [SMILES: OC(c1cncc(Br)c1)C(F)(F)F]

A solution of 5-bromonicotinaldehyde (0.5 g, 2.27 mmol) in THF (10.0 mL) at 0° C. was treated with trimethyl(trifluoromethyl)silane (0.44 mL, 2.95 mmol) and TBAF (0.27 mL, 0.27 mmol) and stirred at RT for 5 h. The mixture was diluted with ice cooled H₂O and extracted with EtOAc, The organic layer was separated, dried (Na₂SO₄) and concentrated. Purification of the residue by column chromatography (30% EtOAc in petroleum ether) gave 1-(5-bromopyridin-3-yl)-2,2,2-trifluoroethan-1-ol as a pale-yellow solid (0.4 g, 58%). MS m/z: 256.01.

### Intermediate 21: 3-bromo-5-(2,2-difluorovinyl)pyridine

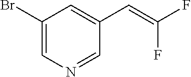 [SMILES: FC(F)=Cc1cncc(Br)c1]

Sodium 2-chloro-2,2-difluoroacetate (0.1.71 g, 13.4 mmol) was added to a stirred solution of 5-bromonicotinaldehyde (1.0 g, 6.7 mmol) and triphenyl phosphine (1.02 g, 7.37 mmol) in DMF (10 mL) at 0° C. The mixture was heated to 100° C. for 1.5 h, quenched with ice H₂O and extracted with Et₂O. The organic layer was dried (Na₂SO₄) and evaporated. Purification of the residue by column chromatography (3-4% EtOAc in petroleum ether) gave 3-bromo-5-(2,2-difluorovinyl)pyridine (0.75 g, 32%) as colourless liquid. ¹H NMR (400 MHz, DMSO-d₆): δ 8.54 (d, J=2.00 Hz, 1H), 8.45 (d, J=2.00 Hz, 1H), 7.84-7.83 (m, 1H), 5.26 (dd, J=2.80, 25.60 Hz, 1H). MS m/z: 220.11.

### Intermediate 22: 2-(5-bromopyridin-3-yl)ethan-1-ol

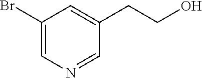

Lithium aluminium hydride (2.4M in THF, 4.0 mL, 10.2 mmol) was added to a stirred solution of 2-(5-bromopyridin-3-yl)acetic acid (1.1 g, 5.1 mmol) in THF at −78° C. The mixture was stirred at RT for 12 h, quenched with sat. aq. Na₂SO₄ at 0° C. and extracted with EtOAc (2×70 mL). The combined organic layers were dried over Na₂SO₄, filtered and concentrated. Purification of the residue by flash chromatography (silica 100-200, 50% EtOAc in petroleum ether) gave 2-(5-bromopyridin-3-yl)ethan-1-ol (0.27 g, 26%) as a solid. ¹H NMR (400 MHz, DMSO-d₆): δ 8.55 (d, J=8.0 Hz, 1H), 8.41 (d, J=8.0 Hz, 1H), 7.75 (s, 1H), 3.90 (t, J=6.40 Hz, 2H), 2.86 (t, J=6.40 Hz, 2H), 1.56 (s, 1H). MS m/z: 201.95.

### Intermediate 23: O-phenyl O-(2,2,2-trifluoro-1-(5-(5-(((R)-1-(4-fluorophenyl)ethyl)amino)pyrazin-2-yl)pyridin-3-yl)ethyl) carbonothioate

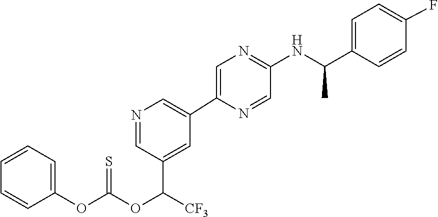 [SMILES: C[C@@H](Nc1cnc(-c2cncc(C(OC(=S)Oc3ccccc3)C(F)(F)F)c2)cn1)c1ccc(F)cc1]

A solution of 2,2,2-trifluoro-1-(5-(5-(((R)-1-(4-fluorophenyl)ethyl)amino)pyrazin-2-yl)pyridin-3-yl)ethan-1-ol (Example 32) (0.50 g, 1.27 mmol) in dry CH₂Cl₂ (10 mL) was treated with O-phenyl carbonochloridothioate (0.33 g, 1.91 mmol) and DMAP (0.467 g, 3.82 mmol) and stirred at RT for 2 h. The mixture was diluted with CH₂Cl₂, washed with ice cold brine (20 mL), dried over Na₂SO₄ and concentrated to obtain crude O-phenyl O-(2,2,2-trifluoro-1-(5-(5-(((R)-1-(4-fluorophenyl)ethyl)amino)pyrazin-2-yl)pyridin-3-yl)ethyl) carbonothioate (750 mg, crude), which was used as such for next step without further purification. MS m/z: 529.34.

### Intermediate 24: (5-(methylsulfonyl)pyridin-3-yl)boronic acid

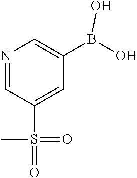

A stirred solution of 3-bromo-5-(methylsulfonyl)pyridine (500 mg, 2.13 mmol) and bis(pinacolato)diboron (810 mg, 3.19 mmol) in dioxane (20 mL) was treated with KOAc (628 mg, 6.38 mmol), purged with N₂ and treated with Pd(dppf)Cl₂ (155 mg, 0.213 mmol). The mixture was stirred at 100° C. for 16 h, cooled to RT and filtered through Celite®. The filtrate was concentrated under reduced pressure to afford crude (5-(methylsulfonyl)pyridin-3-yl)boronic acid as a thick gum (500 mg), which was used for the next step without further purification. MS m/z: 202.00.

### Intermediate 25: (S)-5-chloro-N-(2,3-dihydro-1H-inden-1-yl)pyrazin-2-amine

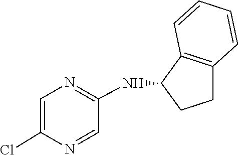 [SMILES: Clc1cnc(N[C@H]2CCc3ccccc32)cn1]

To a stirred solution of (S)-2,3-dihydro-1H-inden-1-amine (180 mg, 1.34 mmol) in N-methyl-2-pyrrolidone was added DIPEA (500 mg, 3.87 mmol) and 2,5-dichloropyrazine (200 mg, 1.34 mmol). The mixture was stirred for 6 h at 170° C., treated with ice cold H₂O and extracted with EtOAc. The combined organic layers were washed with ice cold H₂O, dried over Na₂SO₄ and concentrated. Purification of the residue by column chromatography (Davisil silica, 10-30% EtOAc in petroleum ether) gave (S)-5-chloro-N-(2,3-dihydro-1H-inden-1-yl)pyrazin-2-amine (170 mg, 45%) as a sticky solid. MS m/z: 246.11.

### Intermediate 26: (R)-5-(5-((1-(4-fluorophenyl)ethyl)amino)pyrazin-2-yl)nicotin aldehyde

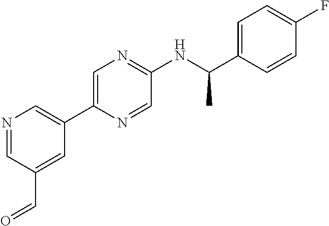 [SMILES: C[C@@H](Nc1cnc(-c2cncc(C=O)c2)cn1)c1ccc(F)cc1]

The title compound was prepared following the procedure employed for Intermediate 2 (step 2) using 5-bromonicotinaldehyde (0.5 g, 1.5 mmol) and (R)-(5-((1-(4-fluorophenyl)ethyl)amino)pyrazin-2-yl)boronic acid (Intermediate 8) (0.509 g, 1.95 mmol). Purification of the crude product by column chromatography gave (R)-5-(5-((1-(4-fluorophenyl)ethyl)amino)pyrazin-2-yl)nicotinaldehyde (0.45 g, 52%) as a white solid. ¹H NMR (400 MHz, CDCl₃): δ 10.17 (s, 1H), 9.32 (d, J=2.00 Hz, 1H), 9.02 (s, 1H), 8.60 (s, 1H), 8.53 (s, 1H), 7.90 (s, 1H), 7.38-7.34 (m, 2H), 7.04 (t, J=8.80 Hz, 2H), 5.16 (d, J=6.40 Hz, 1H), 5.01 (t, J=6.40 Hz, 1H), 1.62 (d, J=6.80 Hz, 3H). MS m/z: 322.95.

### Intermediate 27: 5-bromo-N-(5-fluoro-2,3-dihydro-1H-inden-1-yl)pyridin-2-amine

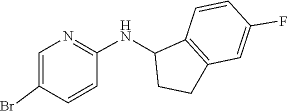 [SMILES: Fc1ccc2c(c1)CCC2Nc1ccc(Br)cn1]

The title compound was prepared following the procedure employed for Intermediate 2 (step 2) using 5-fluoro-2,3-dihydro-1H-inden-1-amine hydrochloride (100 mg, 0.5 mmol) and 5-bromo-2-fluoropyridine (88 mg, 0.5 mmol). Purification of the crude material by column chromatography gave 5-bromo-N-(5-fluoro-2,3-dihydro-1H-inden-1-yl)pyridin-2-amine as a yellow oil (40 mg, 20%). MS m/z: 307.31.

### Intermediate 28: 3-bromo-5-(difluoromethoxy)pyridine

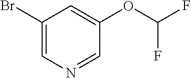 [SMILES: FC(F)Oc1cncc(Br)c1]

A solution of 5-bromopyridin-3-ol (1.0 g, 5.7 mmol) and sodium 2-chloro-2,2-difluoroacetate (1.732 g, 11.4 mmol) in DMF (10 mL and H₂O (1.5 mL) was treated with K₂CO₃ (1.18 g, 8.55 mmol) and heated to 100° C. for 12 h. The mixture was quenched with ice H₂O, extracted with Et₂O and evaporated. Purification of the residue by silica gel column chromatography (7% EtOAc in petroleum ether) gave 3-bromo-5-(difluoromethoxy)pyridine (0.1 g, 1.3%) as a colourless liquid. ¹H NMR (400 MHz, CDCl₃): δ 8.57 (d, J=1.60 Hz, 1H), 8.44 (d, J=2.00 Hz, 1H), 7.68-7.67 (m, 1H), 6.56 (t, J=72.00 Hz, 1H). MS m/z: 225.87.

### Intermediate 29: 3-(2,2-difluorovinyl)-5-(4,4,5,5-tetramethyl-1,3,2-dioxaborolan-2-yl)pyridine

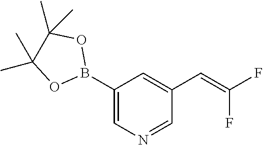 [SMILES: CC1(C)OB(c2cncc(C=C(F)F)c2)OC1(C)C]

The title compound was prepared following the procedure employed for Intermediate 1 (step 2) using 3-bromo-5-(2,2-difluorovinyl)pyridine (Intermediate 21) (350 mg, 1.59 mmol). The crude product, a brown solid (344 mg, 81%), was used for the next step without further purification. MS m/z: 186.02 (boronic acid).

### Intermediate 30: 3-(4,4,5,5-tetramethyl-1,3,2-dioxaborolan-2-yl)-5-(2,2,2-trifluoroethoxy)pyridine

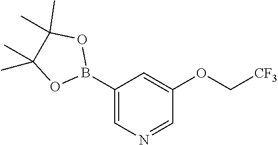 [SMILES: CC1(C)OB(c2cncc(OCC(F)(F)F)c2)OC1(C)C]

Step 1: A solution of 5-bromopyridin-3-ol (200 mg, 1.149 mmol) and 2,2,2-trifluoroethyl trifluoromethanesulfonate (293 mg, 1.26 mmol) in DMF was treated with K₂CO₃ (317 mg, 2.2988 mmol) and stirred for 16 h at RT, diluted with H₂O (i mL) and extracted with EtOAc (3×20 mL). The combined organic layers were washed with ice cold H₂O, dried over Na₂SO₄ and concentrated under reduced pressure to give crude 3-bromo-5-(2,2,2-trifluoroethoxy)pyridine as a pale yellow, sticky solid (300 mg), which was used for the next step without further purification. MS m/z: 255.85.

Step 2: The title compound was prepared following the procedure employed for Intermediate 1 (step 2) using 3-bromo-5-(2,2,2-trifluoroethoxy)pyridine (300 mg, 1.18 mmol), resulting in crude 3-(4,4,5,5-tetramethyl-1,3,2-dioxaborolan-2-yl)-5-(2,2,2-trifluoroethoxy)pyridine (500 mg, crude) as a thick liquid, which was used for the next step without further purification. MS m/z: 221.85 (boronic acid).

### Intermediate 31: O-phenyl O-(2,2,2-trifluoro-1-(5-(3-(((R)-1-(4-fluorophenyl)ethyl)amino)-1,2,4-triazin-6-yl)pyridin-3-yl)ethyl) carbonothioate

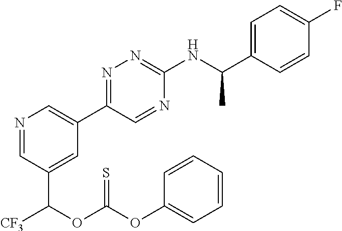 [SMILES: C[C@@H](Nc1ncc(-c2cncc(C(OC(=S)Oc3ccccc3)C(F)(F)F)c2)nn1)c1ccc(F)cc1]

Step 1: 5-(4,4,5,5-tetramethyl-1,3,2-dioxaborolan-2-yl)nicotinaldehyde was prepared following the procedure employed for Intermediate 2 (step 1) using 5-bromonicotinaldehyde (3.0 g, 16.1 mmol). The crude 5-(4,4,5,5-tetramethyl-1,3,2-dioxaborolan-2-yl)nicotinaldehyde was obtained (2.4 g, 64%) as black gum and used without further purification.

Step 2: A solution of (R)-6-chloro-N-(1-(4-fluorophenyl)ethyl)-1,2,4-triazin-3-amine (707 mg, 2.8 mmol) in dioxane (20 mL) and H₂O (5 mL) was treated with 5-(4,4,5,5-tetramethyl-1,3,2-dioxaborolan-2-yl)nicotinaldehyde (1.0 g, 4.2 mmol) and cesium carbonate (2.7 g, 8.4 mmol) under N₂. The mixture was purged for 15 min with N₂, treated with Pd(PPh₃)₄ (323 mg, 0.28 mmol) purged for 15 min with N₂, sealed and stirred at 100° C. for 10 h. The solvent was evaporated and the residue was dissolved in H₂O (10 mL) and extracted with EtOAc. The extracts were combined, dried over Na₂SO₄ and concentrated. Purification of the residue by column chromatography gave (R)-5-(3-((1-(4-fluorophenyl)ethyl)amino)-1,2,4-triazin-6-yl)nicotinaldehyde (450 mg, 50%) as an off-white solid. MS m/z: 324.40.

Step 3: Trimethyl(trifluoromethyl)silane (296 mg, 2.08 mmol) was added to a stirred solution of (R)-5-(3-((1-(4-fluorophenyl)ethyl)amino)-1,2,4-triazin-6-yl)nicotinaldehyde (450 mg, 1.39 mmol) in THF (10 mL). The mixture was treated with TBAF (0.13 mL, 0.13 mmol) at 0° C., stirred at RT for 4 h, quenched with ice-H₂O and extracted with EtOAc (3×40 mL). The combined organic layers were dried over Na₂SO₄ and concentrated. Purification of the residue by column chromatography afforded 2,2,2-trifluoro-1-(5-(3-(((R)-1-(4-fluorophenyl)ethyl)amino)-1,2,4-triazin-6-yl)pyridin-3-yl)ethan-1-ol (250 mg, 45%) as off-white solid. ¹H NMR (400 MHz, DMSO-d₆): δ 9.15 (d, J=2.00 Hz, 1H), 8.92 (s, 1H), 8.71 (s, 1H), 8.49 (s, 1H), 7.64-7.53 (m, 3H), 7.48-7.44 (m, 2H), 7.16-7.11 (m, 2H), 5.41-5.36 (m, 1H), 1.51 (d, J=7.20 Hz, 3H). MS m/z: 394.20.

Step 4: DMAP (192 mg, 1.57 mmol) was added to a stirred solution of 2,2,2-trifluoro-1-(5-(3-(((R)-1-(4-fluorophenyl)ethyl)amino)-1,2,4-triazin-6-yl)pyridin-3-yl)ethan-1-ol (250 mg, 0.63 mmol) and O-phenyl chlorothionoformate (163 mg, 0.94 mmol) in CH₂Cl₂ (10 mL) at 0° C. The resulting mixture was stirred at RT for 4 h, quenched with ice-H₂O and extracted with CH₂Cl₂ (3×20 mL). The combined organic layers were dried over anhydrous Na₂SO₄ and concentrated to afford crude O-phenyl O-(2,2,2-trifluoro-1-(5-(3-(((R)-1-(4-fluorophenyl)ethyl)amino)-1,2,4-triazin-6-yl)pyridin-3-yl)ethyl) carbonothioate as a pale yellow thick liquid (300 mg), which was used without further purification. MS m/z: 530.32.

### Intermediate 32: 3-(4,4,5,5-tetramethyl-1,3,2-dioxaborolan-2-yl)-5-(trifluoromethoxy)pyridine

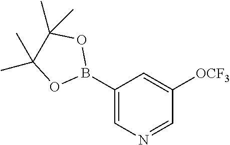 [SMILES: CC1(C)OB(c2cncc(OC(F)(F)F)c2)OC1(C)C]

The title compound was prepared following the procedure employed for Intermediate 1 (step 2) using 3-bromo-5-(trifluoromethoxy)pyridine (100 mg, 0.4 mmol). Crude 3-(4,4,5,5-tetramethyl-1,3,2-dioxaborolan-2-yl)-5-(trifluoromethoxy)pyridine (60 mg, 58%) was obtained as pale yellow thick liquid, which was used without further purification. MS m/z: 207.03 (boronic acid).

### Intermediate 33: 5-chloro-3-vinylpyridazine

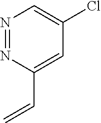

A stirred solution of 3,5-dichloropyridazine (2 g, 13.42 mmol), trifluoro(vinyl)-14-borane (potassium salt, 1.80 g, 13.4 mmol) in propan-2-ol was treated with triethylamine (4.0 g, 40.2 mmol), purged with N₂, treated with Pd(dppf)Cl₂ (980 mg, 1.34 mmol) and heated at 80° C. for 16 h. The mixture was cooled, filtered through Celite® and the filtrate was concentrated under reduced pressure. Purification of the crude by column chromatography (Davisil silica, 0-20% EtOAc:petroleum ether) gave 5-chloro-3-vinylpyridazine as an off-white solid (1.5 g, 72%). ¹H NMR (400 MHz, CDCl₃): δ 9.28 (d, J=2.40 Hz, 1H), 8.26 (d, J=2.40 Hz, 1H), 7.03-6.96 (m, 1H), 6.49 (d, J=17.60 Hz, 1H), 5.80 (d, J=10.80 Hz, 1H). MS m/z: 140.80.

### Intermediate 34: (R)-5-(5-((1-(4-fluorophenyl)ethyl)amino)pyrazin-2-yl)pyridazine-3-carbaldehyde

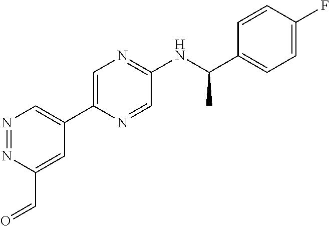 [SMILES: C[C@@H](Nc1cnc(-c2cnnc(C=O)c2)cn1)c1ccc(F)cc1]

A stirred solution of (R)-N-(1-(4-fluorophenyl)ethyl)-5-(6-vinylpyridazin-4-yl)pyrazin-2-amine (Example 44) (850 mg, 2.65 mmol) in acetone:t-butanol:H₂O (1:1:1) was treated with sodium periodate (1.13 g, 5.3 mmol) and osmium tetroxide (5 mL, 5.3 mmol) at 0° C. and stirred for 5 h at 0° C. The mixture was filtered and washed with EtOAc. The filtrate was concentrated and the residue purified by column chromatography (using Davisil silica, 0-15% MeOH in CH₂Cl₂) to give (R)-5-(5-((1-(4-fluorophenyl)ethyl)amino)pyrazin-2-yl)pyridazine-3-carbaldehyde (400 mg, 45%) as pale yellow sticky solid. MS m/z: 322.14.

### Intermediate 35: O-phenyl O-(2,2,2-trifluoro-1-(5-(5-(((R)-1-(4-fluorophenyl)ethyl)amino)pyrazin-2-yl)pyridazin-3-yl)ethyl) carbonothioate

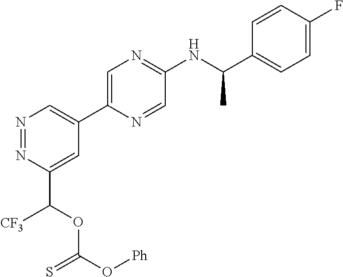 [SMILES: C[C@@H](Nc1cnc(-c2cnnc(C(OC(=S)Oc3ccccc3)C(F)(F)F)c2)cn1)c1ccc(F)cc1]

A stirred solution of 2,2,2-trifluoro-1-(5-(5-(((R)-1-(4-fluorophenyl)ethyl)amino)pyrazin-2-yl)pyridazin-3-yl)ethan-1-ol (Example 58) (100 mg, 0.254 mmol) in CH₂Cl₂ was treated with O-phenyl carbonochloridothioate (66 mg, 0.38 mmol), followed by DMAP (93 mg, 0.76 mmol) at 0° C. and stirred at RT for 2 h. The mixture was diluted with CH₂Cl₂, washed with H₂O, dried over Na₂SO₄ and concentrated under reduced pressure to give crude O-phenyl O-(2,2,2-trifluoro-1-(5-(5-(((R)-1-(4-fluorophenyl)ethyl)amino)pyrazin-2-yl)pyridazin-3-yl)ethyl) carbonothioate, which was used without further purification. MS m/z: 530.35.

### Intermediate 36: (R)-N-(1-(4-fluorophenyl)ethyl)-5-(4,4,5,5-tetramethyl-1,3,2-dioxaborolan-2-yl)pyrimidin-2-amine

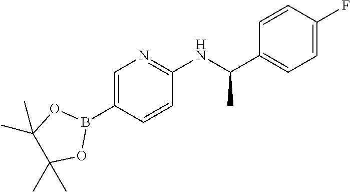 [SMILES: C[C@@H](Nc1ccc(B2OC(C)(C)C(C)(C)O2)cn1)c1ccc(F)cc1]

Step 1: To a stirred solution of 5-bromo-2-chloropyrimidine (1.53 g, 7-90 mmol) and (R)-1-(4-fluorophenyl)ethan-1-amine (1.0 g, 7.18 mmol) in EtOH (10 mL) in a glass tube, was added DIPEA (2.5 mL, 14.36 mmol) at RT under N₂ atmosphere. The tube was sealed and reaction mixture was stirred at 90° C. for 16 h. After this time, the reaction mixture was diluted with H₂O (200 mL) and extracted with EtOAc (2×200 mL). The combined organic layers were dried over anhydrous Na₂SO₄, filtered and concentrated under reduced pressure. The crude product was purified by flash column chromatography (0-50% EtOAc in petroleum ether). This gave (R)-5-bromo-N-(1-(4-fluorophenyl)ethyl)pyrimidin-2-amine as off-white solid (1.1 g, 51%). ¹H NMR (400 MHz, CDCl₃): δ 8.25 (s, 2H), 7.33-7.29 (m, 2H), 7.02-6.98 (m, 2H), 5.42 (d, J=6.40 Hz, 1H), 5.08-5.05 (m, 1H), 1.53 (d, J=7.20 Hz, 3H). MS m/z: 296.08.

Step 2: The title compound was prepared following the procedure employed for Intermediate 1 (step 2) using (R)-5-bromo-N-(1-(4-fluorophenyl)ethyl)pyrimidin-2-amine (0.50 g, 1.69 mmol). Crude (R)-N-(1-(4-fluorophenyl)ethyl)-5-(4,4,5,5-tetramethyl-1,3,2-dioxaborolan-2-yl)pyrimidin-2-amine as black gum was obtained (0.95 g), which was used in the next step without further purification. MS m/z: 344.23.

### Intermediate 37: 6-chloro-3-(5-(2,2,2-trifluoroethyl)pyridin-3-yl)-1,2,4-triazine

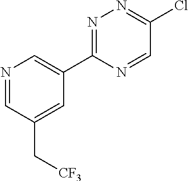 [SMILES: FC(F)(F)Cc1cncc(-c2ncc(Cl)nn2)c1]

Step 1: A solution of ethyl 2-bromo-2,2-difluoroacetate (40 g, 0.19 mol) in MeOH (400 mL) was treated with KOH (11 g, 0.19 mol) and stirred at RT for 24 h. The mixture was evaporated under reduced pressure to obtain a semi-solid, which was co-distilled with toluene three times to afford potassium 2-bromo-2,2-difluoroacetate as a white solid (41 g, 97%), which was used in the next step without further purification.

Step 2: To a stirred solution of potassium 2-bromo-2,2-difluoroacetate (6.0 g, 28 mmol) in DMF (60 mL) was added PPh₃ (7.38 g, 28.16 mmol) and the resulting mixture was stirred at RT for 24 h. The mixture was filtered and the solid obtained was washed with DMF, H₂O and Et₂O successively and then dried under vacuum to afford 2,2-difluoro-2-(triphenylphosphonio)acetate (6.0 g, 60%), which was used in the next step without further purification.

Step 3: A solution of 5-bromonicotinaldehyde (6.0 g, 32 mmol) in DMF (60 mL) was treated with 2,2-difluoro-2-(triphenylphosphonio)acetate (8.90 g, 48.4 mmol), stirred at 80° C. for 1 h, treated with TBAF solution and stirred for 30 min at 80° C. The mixture was cooled to RT, quenched with ice cold H₂O and extracted with Et₂O. The combined organic layers were washed with ice cold H₂O, dried over Na₂SO₄ and concentrated. Purification of the residue by column chromatography (amine silica, 20-30% EtOAc in petroleum ether) gave 3-bromo-5-(2,2,2-trifluoroethyl)pyridine (1.0 g, 13%). ¹H NMR (400 MHz, CDCl₃): δ 8.68 (s, 1H), 8.47 (s, 1H), 7.81 (s, 1H), 3.39 (q, J=10.40 Hz, 2H). MS m/z: 239.86.

Step 4: 3-(4,4,5,5-tetramethyl-1,3,2-dioxaborolan-2-yl)-5-(2,2,2-trifluoroethyl)pyridine was prepared following the procedure employed for Intermediate 1 (step 2) using 3-bromo-5-(2,2,2-trifluoroethyl)pyridine (1.0 g, 4.1 mmol). 3-(4,4,5,5-tetramethyl-1,3,2-dioxaborolan-2-yl)-5-(2,2,2-trifluoroethyl)pyridine (1.1 g, 76%) was obtained as pale brown thick liquid, which was used in the next step without further purification MS m/z: 205.69 (boronic acid).

Step 5: A solution of 3-bromo-1,2,4-triazine 2-oxide (686 mg, 3.89 mmol) in dioxane (40 mL) and H₂O (4 mL) was treated with 3-(4,4,5,5-tetramethyl-1,3,2-dioxaborolan-2-yl)-5-(2,2,2-trifluoroethyl)pyridine (1.1 g, 4.2 mmol), K₂CO₃ (1.6 g, 11.67 mmol), purged with N₂, treated with Pd(amphos)Cl₂ (269 mg, 0.38 mmol) and heated at 100° C. for 8 h. The mixture was cooled to RT and filtered through Celite®. The filtrated was concentrated and the residue purified by reverse phase column chromatography to afford 3-(5-(2,2,2-trifluoroethyl)pyridin-3-yl)-1,2,4-triazine 2-oxide (65 mg, 7%) as pale yellow thick liquid, which was used in the next step without further purification. MS m/z: 257.20.

Step 6: A mixture of 3-(5-(2,2,2-trifluoroethyl)pyridin-3-yl)-1,2,4-triazine-2-oxide (65 mg, 0.253 mmol) and POCl₃ (10 mL) was heated for 80° C. for 5 h, quenched with ice-cold NaHCO₃ solution and extracted with CHCl₃. The organic layer was dried over Na₂SO₄ and concentrated under reduced pressure to obtain crude 6-chloro-3-(5-(2,2,2-trifluoroethyl)pyridin-3-yl)-1,2,4-triazine (27 mg, 39%) as pale yellow thick liquid, which was used without further purification. MS m/z: 274.96.

### Intermediate 8: (R)-6-chloro-N-(1-(4-fluorophenyl)ethyl)-1,2,4-triazin-3-amine

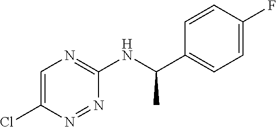 [SMILES: C[C@@H](Nc1ncc(Cl)nn1)c1ccc(F)cc1]

Step 1: To a stirred solution of 1,2,4-triazin-3-amine (500 mg, 5.2 mmol) in CH₃CN (10 mL) was added m-CPBA (1.17 g, 6.76 mmol) and the mixture was stirred for 6 h at 70° C. The solvent was evaporated and the residue triturated with Et₂O (5×100 mL), filtered and dried under vacuum to give 3-amino-1,2,4-triazine 2-oxide as off-white solid (500 mg, 89%), which was used without further purification. MS m/z: 112.96 (M+H).

Step 2: To a stirred slurry of 3-amino-1,2,4-triazine 2-oxide (500 mg, 4.46 mmol) in HBr in H₂O (20 mL) was added NaNO₂ (1.5 g, 22.32 mmol, in 2 mL H₂O) slowly at 0° C. and the mixture was stirred for 1 h at RT. The solvent was evaporated from the mixture and the residue dissolved in DCM, washed with aq. NaHCO₃ solution (5 mL), dried over Na₂SO₄ and concentrated under reduced pressure to give 3-bromo-1,2,4-triazine 2-oxide as a brown solid (300 mg, 38%). ¹H NMR (400 MHz, DMSO-d₆): δ 8.82 (s, 1H), 7.88 (s, 1H). MS m/z: 175.94 (M+H).

Step 3: A stirred solution of 3-bromo-1,2,4-triazine 2-oxide (300 mg, 1.7 mmol) and (R)-1-(4-fluorophenyl)ethan-1-amine (248 mg, 1.78 mmol) in dioxane (10 mL) was treated with K₂CO₃ (704 mg, 5.1 mmol) and stirred for 1 h at 100° C. The solvent was evaporated under reduced pressure to give a residue which was triturated with Et₂O and decanted off to obtain crude (R)-3-((1-(4-fluorophenyl)ethyl)amino)-1,2,4-triazine 2-oxide as a thick liquid (600 mg), which was used without further purification. MS m/z: 235.12.

Step 4: Hydrogen chloride gas was purged to a stirred solution of (R)-3-((1-(4-fluorophenyl)ethyl)amino)-1,2,4-triazine 2-oxide (600 mg, 2.4 mmol) in CHCl₃ for 3 h at RT and the mixture was stirred for 16 h at RT. The mixture was neutralized with Na₂CO₃ to pH˜8, stirred for 15 min at RT, and filtered. The cake was washed with CHCl₃ and the filtrate concentrated to give (R)-6-chloro-N-(1-(4-fluorophenyl)ethyl)-1,2,4-triazin-3-amine (250 mg, 39%). ¹H NMR (400 MHz, DMSO-d₆): δ 8.58 (br s, 1H), 8.40 (s, 1H), 7.43-7.40 (dd, J=2.0 Hz and 8.4 Hz, 2H), 7.13 (t, J=8.8 Hz, 2H), 5.07 (br s, 1H), 1.17 (d, J=7.2 Hz, 3H). MS m/z: 253.08 (M+H).

### Intermediate 39: (R)-5-chloro-N-(2,3-dihydro-1H-inden-1-yl)pyrazin-2-amine

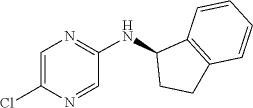 [SMILES: Clc1cnc(N[C@@H]2CCc3ccccc32)cn1]

A stirred solution of (R)-2,3-dihydro-1H-inden-1-amine (200 mg, 1.50 mmol) in NMP was treated with DIPEA (0.59 g, 4.5 mmol) and 2,5-dichloropyrazine (223 mg, 1.50 mmol) and stirred for 6 h at 170° C. The mixture was treated with ice cold H₂O and extracted with EtOAc. The organic layer was dried over Na₂SO₄ and concentrated under reduced pressure. Purification of the residue by column chromatography (Davisil silica, 10-30% EtOAc in petroleum ether) gave (R)-5-chloro-N-(2,3-dihydro-1H-inden-1-yl)pyrazin-2-amine (140 mg, 38%) as a sticky solid. MS m/z: 246.13.

### Intermediate 40: 3-methyl-5-(4,4,5,5-tetramethyl-1,3,2-dioxaborolan-2-yl)benzo[d]oxazol-2(3H)-one

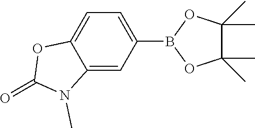 [SMILES: Cn1c(=O)oc2ccc(B3OC(C)(C)C(C)(C)O3)cc21]

Step 1: A stirred solution of 5-bromobenzo[d]oxazol-2(3H)-one (0.10 g, 0.46 mmol) in DMF (2 mL) was treated with K₂CO₃ (0.39 g, 2.80 mmol) and Mel (0.2 g, 1.4 mmol) successively. The reaction mixture was stirred at RT under N₂ for 1 h and diluted with EtOAc (50 mL) and H₂O (50 mL). The layers were separated and the organic layer was washed with H₂O (3×50 mL), dried over anhydrous Na₂SO₄, filtered and concentrated under reduced pressure to obtain 5-bromo-3-methylbenzo[d]oxazol-2(3H)-one as pale brown solid (0.1 g, 94% yield). ¹H NMR (400 MHz, CDCl₃): δ 7.26-7.24 (m, 1H), 7.11-7.06 (m, 2H), 3.39 (s, 3H). MS m/z: 228.10.

Step 2: A stirred solution of 5-bromo-3-methylbenzo[d]oxazol-2(3H)-one (3 g, 13.1 mmol) in dioxane (130 mL) was treated with bis(pinacolato)diboron (4.90 g, 19.7 mmol), KOAc (3.8 g, 39 mmol), purged with N₂, treated with Pd(dppf)Cl₂ (958 mg, 1.31 mmol) and stirred for 2 h at 100° C. The mixture was filtered through Celite® and the filtrate concentrated. The residue was triturated with n-pentane and decanted. The supernatant was concentrated to give 3-methyl-5-(4,4,5,5-tetramethyl-1,3,2-dioxaborolan-2-yl)benzo[d]oxazol-2(3H)-one (2.9 g, 80%) as pale yellow thick liquid, which was used without further purification. MS m/z: 193.91 (boronic acid), 276.26 (boronic ester).

### Intermediate 41: 3-(2-hydroxyethyl)-5-(4,4,5,5-tetramethyl-1,3,2-dioxaborolan-2-yl)benzo[d]oxazol-2(3H)-one

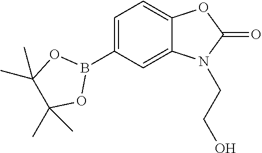 [SMILES: CC1(C)OB(c2ccc3oc(=O)n(CCO)c3c2)OC1(C)C]

Step 1: A suspension of 5-bromo benzo[d]oxazol-2(3H)-one (1 g, 4.7 mmol) and K₂CO₃ (1.29 g, 9.4 mmol) in DMF (20 mL) was treated with 2-bromoethanol (800 mg, 5.64 mmol) and stirred for 2 h at RT. The mixture was quenched with ice-water and extracted with EtOAc. The organic layer was dried (Na₂SO₄) and concentrated under vacuum to give 5-bromo-3-(2-hydroxyethyl)benzo[d]oxazol-2(3H)-one as a yellow solid (1.1 g, 80%). ¹H NMR (400 MHz, CDCl₃): δ 7.26-7.23 (m, 2H), 7.08 (d, J=8.0 Hz, 1H), 4.01-3.96 (m, 4H), 1.94 (t, J=4.80 Hz, 1H). MS m/z: 259.95.

Step 2: 3-(2-hydroxyethyl)-5-(4,4,5,5-tetramethyl-1,3,2-dioxaborolan-2-yl)benzo[d]oxazol-2(3H)-one was prepared following the procedure employed for Intermediate 1 (step 2) using 5-bromo-3-(2-hydroxyethyl)benzo[d]oxazol-2(3H)-one (500 mg, 1.93 mmol). Heating for 12 h gave the title compound as a pale brown thick gum (230 mg, 39%), which was used without further purification. MS m/z: 306.43.

### Intermediate 42: 3-ethyl-5-(4,4,5,5-tetramethyl-1,3,2-dioxaborolan-2-yl)benzo[d]oxazol-2(3H)-one

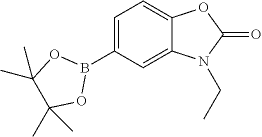 [SMILES: CCn1c(=O)oc2ccc(B3OC(C)(C)C(C)(C)O3)cc21]

Step 1: 5-bromo-3-ethylbenzo[d]oxazol-2(3H)-one was prepared according to the procedure employed for Intermediate 41 (step 1) using 5-bromo benzo[d]oxazol-2(3H)-one (0.35 g, 1.63 mmol) and ethyl iodide (0.37 g, 2.4 mmol). The desired compound was obtained as a pale-yellow solid ((0.37 g, 94%), which was used without further purification. MS m/z: 242.26.

Step 2: The title compound was prepared following the procedure employed for Intermediate 40 (step 2) using 5-bromo-3-ethylbenzo[d]oxazol-2(3H)-one (370 mg, 1.50 mmol). 3-Ethyl-5-(4,4,5,5-tetramethyl-1,3,2-dioxaborolan-2-yl)benzo[d]oxazol-2(3H)-one was obtained as a pale yellow gum (300 mg, 70%), which was used without further purification. MS m/z: 289.30 (M+H)

### Intermediate 43: 3-(difluoromethyl)-5-(4,4,5,5-tetramethyl-1,3,2-dioxaborolan-2-yl)benzo[d]oxazol-2(3H)-one

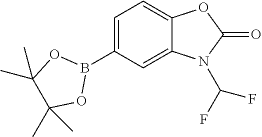 [SMILES: CC1(C)OB(c2ccc3oc(=O)n(C(F)F)c3c2)OC1(C)C]

Step 1: A stirred solution of 5-bromobenzo[d]oxazol-2(3H)-one (500 mg, 2.36 mmol) in DMF was treated with K₂CO₃ (650 mg, 4.72 mmol) and sodium 2-chloro-2,2-difluoroacetate (538 mg, 3.54 mmol) at 0° C. The reaction mixture was stirred for 3 h at 70° C. The mixture was quenched with H₂O and extracted with EtOAc (3×20 mL). The combined organic layers were washed with brine, dried over Na₂SO₄ and concentrated. Purification of the residue by column chromatography (20-30% EtOAc in petroleum ether) gave 5-bromo-3-(difluoromethyl)benzo[d]oxazol-2(3H)-one as a brown solid (350 mg, 57%). ¹H NMR (400 MHz, DMSO-d₆): δ 7.87-7.58 (m, 2H), 7.51-7.44 (m 2H).

Step 2: The title compound was prepared following the procedure employed for Intermediate 1 (step 2) using 5-bromo-3-(difluoromethyl)benzo[d]oxazol-2(3H)-one (350 mg, 1.13 mmol). 3-(difluoromethyl)-5-(4,4,5,5-tetramethyl-1,3,2-dioxaborolan-2-yl)benzo[d]oxazol-2(3H)-one was obtained as a pale yellow liquid, which was used without further purification.

### Intermediate 44: 3-((methylthio)methyl)-5-(4,4,5,5-tetramethyl-1,3,2-dioxaborolan-2-yl)benzo[d]oxazol-2(3H)-one

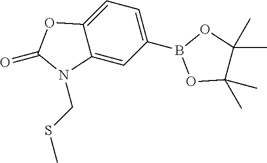 [SMILES: CSCn1c(=O)oc2ccc(B3OC(C)(C)C(C)(C)O3)cc21]

Step 1: A stirred suspension of 5-bromobenzo[d]oxazol-2(3H)-one (1 g, 4.7 mmol) and caesium carbonate (4.59 g, 14.1 mmol) in DMF (10 mL) was treated with (chloromethyl)(methyl)sulfide (1.15 g, 11.75 mmol) and heated at 60° C. for 24 h. The mixture was quenched with ice-water and extracted with EtOAc (3×20 mL). The organic layers were dried over Na₂SO₄ and concentrated in vacuo. The crude product was purified by column chromatography (10% EtOAc/petroleum ether) to give 5-bromo-3-((methylthio)methyl)benzo[d]oxazol-2(3H)-one as a white solid (500 mg, 38%). ¹H NMR (400 MHz, DMSO-d₆): δ 7.69 (s, 1H), 7.35 (s, 2H), 5.03 (s, 2H), 2.13 (s, 3H).

Step 2: The title compound was prepared following the procedure employed for Intermediate 1 (step 2) using 5-bromo-3-((methylthio)methyl)benzo[d]oxazol-2(3H)-one (250 mg, 0.9 mmol). 3-((Methylthio)methyl)-5-(4,4,5,5-tetramethyl-1,3,2-dioxaborolan-2-yl)benzo[d]oxazol-2(3H)-one was obtained as pale yellow thick liquid (280 mg, 99%), which was used without further purification. MS m/z: 237.91 (boronic acid).

### Intermediate 45: 3-((methylsulfonyl)methyl)-5-(4,4,5,5-tetramethyl-1,3,2-dioxaborolan-2-yl)benzo[d]oxazol-2(3H)-one

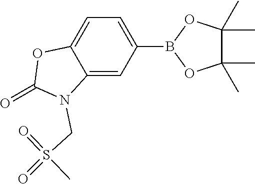 [SMILES: CC1(C)OB(c2ccc3oc(=O)n(CS(C)(=O)=O)c3c2)OC1(C)C]

Step 1: At 0° C., a solution of 5-bromo-3-((methylthio)methyl)benzo[d]oxazol-2(3H)-one (1.35 g, 4.92 mmol) in THF (30 mL) was treated with a solution of oxone (5.60 g, 18.2 mmol) in H₂O (15 mL) and stirred at RT for 36 h. The solvent was evaporated from the mixture and the precipitated solid was filtered, washed with pentane and dried to obtain 5-bromo-3-((methylsulfonyl)methyl)benzo[d]oxazol-2(3H)-one as a white solid. (980 mg, 59%). ¹H NMR (400 MHz, DMSO-d₆): δ 7.70 (s, 1H), 7.38 (s, 2H), 5.43 (s, 2H), 3.13 (s, 3H). MS m/z: 304.11.

Step 2: 3-((methylsulfonyl)methyl)-5-(4,4,5,5-tetramethyl-1,3,2-dioxaborolan-2-yl)benzo[d]oxazol-2(3H)-one was prepared following the procedure employed for Intermediate 1 (step 2) using 5-bromo-3-((methylsulfonyl)methyl)benzo[d]oxazol-2(3H)-one (1.16 g, 3.7 mmol). The crude compound was re-crystallized from Et₂O/n-pentane to furnish the desired compound as a white solid (730 mg, 67%), which was used without further purification. MS m/z: 354.12.

### Intermediate 46: 5-(3-chloro-1,2,4-triazin-6-yl)-3-methylbenzo[d]oxazol-2(3H)-one

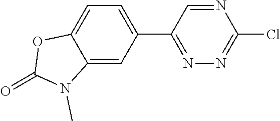 [SMILES: Cn1c(=O)oc2ccc(-c3cnc(Cl)nn3)cc21]

Step 1: A stirred solution of 6-bromo-3-amino-1,2,4-triazine (1.06 g, 6.06 mmol), 3-methyl-5-(4,4,5,5-tetramethyl-1,3,2-dioxaborolan-2-yl)benzo[d]oxazol-2(3H)-one (2 g, 7.27 mmol) in dioxane (40 mL) in a glass tube was treated with K₂CO₃ (2.5 g, 18 mmol) and H₂O (5 mL). The reaction mixture was purged with N₂, treated with Pd(amphos)Cl₂ (0.429 g, 0.606 mmol), sealed and stirred at 100° C. for 3 h. The solvent was evaporated and Et₂O was added to the residue, which was stirred for 10 min, filtered and washed with Et₂O to obtain 5-(3-amino-1,2,4-triazin-6-yl)-3-methylbenzo[d]oxazol-2(3H)-one (5 g, crude) as a black solid, which was used without further purification. MS m/z: 244.06.

Step 2: To a stirred slurry of 5-(3-amino-1,2,4-triazin-6-yl)-3-methylbenzo[d]oxazol-2(3H)-one (1.3 g, 5.3 mmol) in CH₃CN (200 mL) was added tert-butyl nitrite (0.873 g, 8.48 mmol) and copper(II) chloride (0.923 g, 6.89 mmol). The mixture was stirred at 80° C. for 16 h, filtered and the solids washed with CH₃CN. The filtrate and washings were combined and concentrated under reduced pressure to give the crude product. Purification by column chromatography (Davisil silica, 0-20% DCM in MeOH) gave 5-(3-chloro-1,2,4-triazin-6-yl)-3-methylbenzo[d]oxazol-2(3H)-one as a sticky solid, which was used without further purification. MS m/z: 262.95.

### Intermediate 47: 3-(methoxymethyl)-5-(4,4,5,5-tetramethyl-1,3,2-dioxaborolan-2-yl)benzo[d]oxazol-2(3H)-one

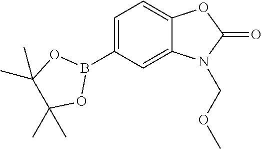 [SMILES: COCn1c(=O)oc2ccc(B3OC(C)(C)C(C)(C)O3)cc21]

Step 1: 5-bromo-3-(methoxymethyl)benzo[d]oxazol-2(3H)-one was prepared following the procedure employed for Intermediate 41 (step 1) using 5-bromo benzo[d]oxazol-2(3H)-one (0.35 g, 1.63 mmol) and chloromethyl methyl ether (0.26 g, 3.27 mmol). The desired compound was obtained as a thick gum (0.27 g, 65%), which was used without further purification. MS m/z: 258.08 (M+H).

Step 2: 3-(methoxymethyl)-5-(4,4,5,5-tetramethyl-1,3,2-dioxaborolan-2-yl)benzo[d]oxazol-2(3H)-one was prepared following the procedure employed for Intermediate 40 (step 2) using 5-bromo-3-(methoxymethyl)benzo[d]oxazol-2(3H)-one (270 mg, 1.06 mmol). The title compound was obtained as pale yellow thick gum (300 mg, crude), which was used without further purification. ¹H NMR (400 MHz, DMSO-d₆): δ 7.65 (d, J=8.0 Hz, 1H), 7.57 (s, 1H), 7.22 (d, J=8.0 Hz, 1H), 5.26 (s, 2H), 3.41 (s, 3H), 1.35 (s, 12H). MS m/z: 305.47.

### Intermediate 48: 6-bromo-3,3-difluoro-1-methylindolin-2-one

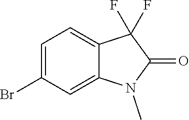

To a stirred solution of 6-bromo-3,3-difluoroindolin-2-one (1.2 g, 4.83 mmol) in DMF (2 mL) was added caesium carbonate (2.55 g, 7.25 mmol) and iodomethane (1.28 g, 7.245 mmol) respectively at room temperature, under a nitrogen atmosphere. After 1 h, the reaction mixture was diluted with EtOAc (50 mL) and water (50 mL). Layers were separated and the organic layer was washed with water (3×50 mL), dried over anhydrous Na₂SO₄, filtered and concentrated under reduced pressure to give 6-bromo-3,3-difluoro-1-methylindolin-2-one as a faint brown solid (1 g, 78%). ¹H NMR (400 MHz, DMSO-d₆): δ 7.41 (d, J=8.0 Hz, 1H), 7.33 (d, J=8.0 Hz, 1H), 7.07 (d, J=1.6 Hz, 1H), 3.21 (s, 3H). MS m/z: 261.99 (M+H).

### Intermediate 49: 1-methyl-6-(4,4,5,5-tetramethyl-1,3,2-dioxaborolan-2-yl)-1H-benzo[d]imidazole

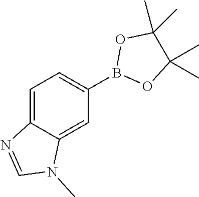 [SMILES: Cn1cnc2ccc(B3OC(C)(C)C(C)(C)O3)cc21]

To a stirred solution of 6-bromo-1-methyl-1H-benzo[d]imidazole (300 mg, 1.42 mmol) and bis(pinacolato)diboron (542 mg, 2.13 mmol) in 1,4-dioxane (10 mL) in a glass tube was added potassium acetate (420 mg, 4.26 mmol). The reaction mixture was purged with N₂ and then Pd(dppf)Cl₂ (104 mg, 0.142 mmol) was added. The tube was sealed and stirred at 100° C. for 16 h and the reaction progress was monitored by TLC and LCMS. After completion of the reaction, the mixture was cooled 25-30° C., filtered through a Celite® bed and washed with ethyl acetate (30 mL). The combined organic layers were concentrated under reduced pressure to afford the crude material as a thick gummy liquid. The crude was purified by column chromatography by using florisil, eluting with 0-40% Ethyl Acetate/Pet ether. The desired fractions were collected and concentrated under reduced pressure to afford 1-methyl-6-(4,4,5,5-tetramethyl-1,3,2-dioxaborolan-2-yl)-1H-benzo[d]imidazole (200 mg, 78%). MS m/z: 259.30 (M+H), 93.90%.

### Intermediate 50: (1-methyl-1H-imidazo[4,5-b]pyridin-6-yl)boronic acid

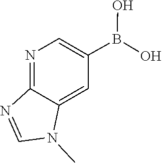

Step 1: To a stirred solution of 6-bromo-3H-imidazo[4,5-b]pyridine (500 mg, 2.52 mmol) in DMSO was added 1M NaHMDS in THF (3.8 mL, 3.8 mmol) and Mel (1.07 g, 7.57 mmol) at 0° C. The mixture was stirred for 3 h at RT, quenched with NH₄Cl solution and extracted with EtOAc. The combined organic layers were washed with brine solution, dried over sodium sulphate and concentrated under reduced pressure to afford the crude material. The crude was purified by column chromatography, eluting with 20-30% EtOAc in Pet ether to collect 6-bromo-3-methyl-3H-imidazo[4,5-b]pyridine (190 mg, 35%) and 100% EtOAc to collect 6-bromo-1-methyl-1H-imidazo[4,5-b]pyridine (160 mg, 30%).

6-bromo-3-methyl-3H-imidazo[4,5-b]pyridine: ¹H NMR (400 MHz, DMSO) δ 8.49 (s, 1H), 8.47 (d, J=2.0 Hz, 1H), 8.37 (d, J=2.0 Hz, 1H), 3.84 (s, 3H). MS m/z: 211.89 (M+H), 99.69%.

6-bromo-1-methyl-1H-imidazo[4,5-b]pyridine: 8.48 (d, J=2.4 Hz, 1H), 8.47 (s, 1H), 8.41 (d, J=2.0 Hz, 1H), 3.86 (s, 3H); MS m/z: 211.89 (M+H), 85.79%.

Step 2: To a stirred solution of 6-bromo-1-methyl-1H-imidazo[4,5-b]pyridine (160 mg, 0.758 mmol), bis(pinacolato)diboron (289 mg, 1.13 mmol) in 1,4-dioxane (10 mL) in a glass tube was added potassium acetate (224 mg, 2.27 mmol). The mixture was purged with N₂ and Pd(dppf)Cl₂ was added (55 mg, 0.076 mmol). The tube was sealed and stirred at 100° C. for 16 h. The mixture was cooled to 25-30° C., filtered through a Celite® bed and washed with ethyl acetate (30 mL). The combined organic layers were concentrated under reduced pressure to afford the crude as a thick gummy liquid. The crude was purified by column chromatography on florisil, eluting with 0-40% EtOAc in Pet ether. The desired fractions were collected and concentrated under reduced pressure to give (1-methyl-1H-imidazo[4,5-b]pyridin-6-yl)boronic acid (160 mg, 90%). MS m/z: 178.21 (boronic acid) (M+H).

### Intermediate 51: (3-methyl-3H-imidazo[4,5-b]pyridin-6-yl)boronic acid

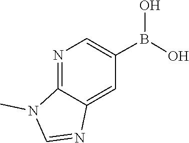

To a stirred solution of 6-bromo-3-methyl-3H-imidazo[4,5-b]pyridine (160 mg, 0.758 mmol), bis(pinacolato)diboron (289 mg, 1.13 mmol) in 1,4-dioxane (10 mL) in a glass tube was added potassium acetate (224 mg, 2.27 mmol). The reaction mixture was purged with N₂ and then Pd(dppf)Cl₂ (55 mg, 0.076 mmol) was added. The tube was sealed and stirred at 100° C. for 16 h and reaction progress was monitored by TLC and LCMS. After completion of the reaction, the mixture was cooled to 25-30° C., filtered through a Celite® bed and washed with ethyl acetate (30 mL). The combined organic layers were concentrated under reduced pressure to afford the crude as a thick gummy liquid. The crude was purified by column chromatography by using florsil silica, eluting with 0-40% Ethyl Acetate/Pet ether. The desired fractions were collected and concentrated under reduced pressure to afford (3-methyl-3H-imidazo[4,5-b]pyridin-6-yl)boronic acid (160 mg, 90%). MS m/z: 178.21 (M+H).

### 2. Synthetic Examples

### Example 1: N-(1-(4-fluorophenyl)ethyl)-5′-methyl-[3,3′-bipyridin]-6-amine

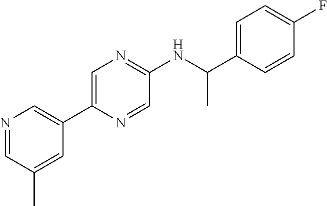 [SMILES: Cc1cncc(-c2cnc(NC(C)c3ccc(F)cc3)cn2)c1]

A solution of 3-bromo-5-methylpyridine (0.2 g, 1.2 mmol), N-(1-(4-fluorophenyl)ethyl)-5-(4,4,5,5-tetramethyl-1,3,2-dioxaborolan-2-yl)pyridin-2-amine (Intermediate 1) (0.616 g, 1.80 mmol) and K₂CO₃ (0.33 g, 2.4 mmol) in dioxane (3.6 ml) and H₂O (0.4 ml) in a glass tube was purged with N₂ for 10 min. PdCl₂(dppf) (0.088 g, 0.1 mmol) was added under N₂ atmosphere; the tube was sealed and the mixture stirred at 100° C. for 16 h. The mixture was diluted with EtOAc (50 ml) and filtered through a Celite® bed. The bed was washed with EtOAc (3×25 ml) and the combined filtrate and washings were washed with H₂O (100 mL). The organic layer was dried over anhydrous Na₂SO₄, filtered and concentrated under reduced pressure to obtain crude product. Purification by reverse phase column chromatography (0-80% MeOH in 0.1% Ammonium bicarbonate in H₂O) afforded N-(1-(4-fluorophenyl)ethyl)-5′-methyl-[3,3′-bipyridin]-6-amine as pale yellow gum (132 mg, 37%). ¹H NMR (400 MHz, DMSO-d₆): δ 8.85 (s, 1H), 8.55 (s, 1H), 8.40 (d, J=1.8 Hz, 1H), 8.34 (s, 1H), 8.09-8.00 (m, 2H), 7.45-7.42 (m, 2H), 7.16 (t, J=8.8 Hz, 1H), 6.81 (d, J=8.8 Hz, 1H), 5.10 (s, 1H), 2.43 (s, 3H), 1.48 (d, J=6.8 Hz, 3H). MS m/z: 308.27.

### Example 2: (R)-N-(1-(3,4-difluorophenyl)ethyl)-5′-methyl-[3,3′-bipyridin]-6-amine

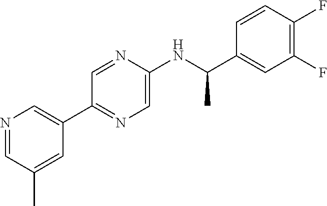 [SMILES: Cc1cncc(-c2cnc(N[C@H](C)c3ccc(F)c(F)c3)cn2)c1]

A solution of 6′-chloro-5-methyl-3,3′-bipyridine (Intermediate 2) (50 mg, 0.244 mmol), (R)-1-(3,4-difluorophenyl)ethan-1-amine (46 mg, 0.293 mmol) and Cs₂CO₃ (159 mg, 0.488 mmol) in toluene (1 mL) was purged with N₂ for 10 min. rac-BINAP (15 mg, 0.0244 mmol) and Pd₂(dba)₃ (11 mg, 0.0122 mmol) were added and the resulting mixture was stirred at 100° C. for 16 h in a sealed tube. The reaction mixture was cooled, diluted with EtOAc (50 mL) and filtered through Celite®. The Celite® bed was washed with EtOAc (2×50 mL) and the combined organic layers were washed with H₂O (100 mL), dried over anhydrous Na₂SO₄, filtered and concentrated under reduced pressure. The crude product was purified by reverse phase column chromatography (0-80% MeOH in 0.1% Ammonium bicarbonate in H₂O) to give (R)-N-(1-(3,4-difluorophenyl)ethyl)-5′-methyl-[3,3′-bipyridin]-6-amine as pale yellow gum (36 mg, 45%). ¹H NMR (400 MHz, DMSO-d₆): δ 8.58 (d, J=1.7 Hz, 1H), 8.29-8.28 (m, 2H), 7.77-7.73 (m, 2H), 7.44-7.23 (m, 4H), 6.61 (d, J=8.7 Hz, 1H), 5.10-5.03 (m, 1H), 2.32 (s, 3H), 1.43 (d, J=6.9 Hz, 3H). HPLC Purity: 99.7%. MS m/z: 326.21.

Using the method employed in the preparation of Example 1, the following compounds were prepared:

### Example 3: (R)-N-(1-(4-fluorophenyl)ethyl)-5′-methyl-[3,3′-bipyridin]-6-amine

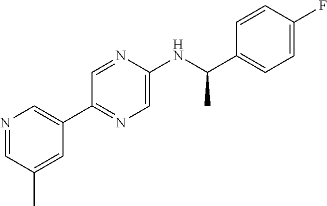 [SMILES: Cc1cncc(-c2cnc(N[C@H](C)c3ccc(F)cc3)cn2)c1]

Using 3-bromo-5-methylpyridine (75 mg, 0.436 mmol) and (R)-N-(1-(4-fluorophenyl)ethyl)-5-(4,4,5,5-tetramethyl-1,3,2-dioxaborolan-2-yl)pyridin-2-amine (Intermediate 3) (179 mg, 0.523 mmol) the crude product was obtained. The crude product was purified by flash column chromatography to give (R)-N-(1-(4-fluorophenyl)ethyl)-5′-methyl-[3,3′-bipyridin]-6-amine as a brown solid (25 mg, 18%). ¹H NMR (400 MHz, DMSO-d₆): δ 8.57 (d, J=1.9 Hz, 1H), 8.29 (s, 2H), 7.76-7.71 (m, 2H), 7.43-7.39 (m, 2H), 7.26 (d, J=7.7 Hz, 1H), 7.12 (t, J=8.9 Hz, 2H), 6.58 (d, J=8.7 Hz, 1H), 5.08-5.05 (m, 1H), 2.31 (s, 3H), 1.43 (d, J=6.9 Hz, 3H). MS m/z: 308.22.

### Example 4: (R)-5′-ethyl-N-(1-(4-fluorophenyl)ethyl)-[3,3′-bipyridin]-6-amine

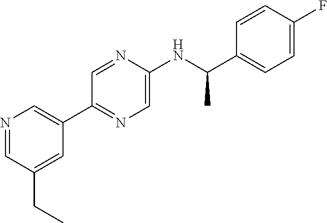 [SMILES: CCc1cncc(-c2cnc(N[C@H](C)c3ccc(F)cc3)cn2)c1]

Using 3-bromo-5-ethylpyridine (50 mg, 0.27 mmol) and (R)-N-(1-(4-fluorophenyl)ethyl)-5-(4,4,5,5-tetramethyl-1,3,2-dioxaborolan-2-yl)pyridin-2-amine (Intermediate 3) (140 mg, 0.403 mmol) the crude product was obtained. The crude was purified by flash column chromatography to give (R)-5′-ethyl-N-(1-(4-fluorophenyl)ethyl)-[3,3′-bipyridin]-6-amine as a pale yellow gum (17 mg, 19%). ¹H NMR (400 MHz, DMSO-d₆): δ 8.59 (d, J=2.1 Hz, 1H), 8.32-8.29 (m, 2H), 7.78-7.72 (m, 2H), 7.43-7.39 (m, 2H), 7.26 (d, J=7.8 Hz, 1H), 7.12 (t, J=8.9 Hz, 2H), 6.59 (d, J=8.7 Hz, 1H), 5.10-5.03 (m, 1H), 2.64 (q, J=7.6 Hz, 2H), 1.43 (d, J=6.9 Hz, 3H), 1.22 (t, J=7.6 Hz, 3H). MS m/z: 322.22.

### Example 5: (R)-5′-cyclopropyl-N-(1-(4-fluorophenyl)ethyl)-[3,3′-bipyridin]-6-amine

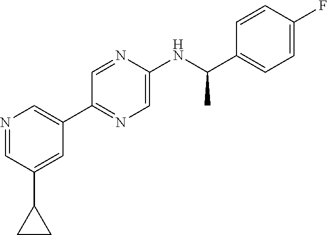 [SMILES: C[C@@H](Nc1cnc(-c2cncc(C3CC3)c2)cn1)c1ccc(F)cc1]

Using 3-bromo-5-cyclopropylpyridine (50 mg, 0.25 mmol) and (R)-N-(1-(4-fluorophenyl)ethyl)-5-(4,4,5,5-tetramethyl-1,3,2-dioxaborolan-2-yl)pyridin-2-amine (Intermediate 3) (104 mg, 0.3 mmol), crude product was obtained. Purification by flash column chromatography gave (R)-5′-cyclopropyl-N-(1-(4-fluorophenyl)ethyl)-[3,3′-bipyridin]-6-amine as a white gum (17 mg, 20%). ¹H NMR (400 MHz, DMSO-d₆): δ 8.52 (d, J=2.1 Hz, 1H), 8.29-8.27 (m, 2H), 7.73 (dd, J=2.40, 8.60 Hz, 1H), 7.50-7.49 (m, 1H), 7.42-7.39 (m, 2H), 7.26 (d, J=7.7 Hz, 1H), 7.12 (t, J=8.9 Hz, 2H), 6.58 (d, J=8.7 Hz, 1H), 5.09-5.02 (m, 1H), 1.98-1.92 (m, 1H), 1.43 (d, J=6.9 Hz, 3H), 1.00-0.96 (m, 2H), 0.83-0.81 (m, 2H). MS m/z: 334.24.

### Example 6: (R)-N-(1-(4-fluorophenyl)ethyl)-5′-methoxy-[3,3′-bipyridin]-6-amine

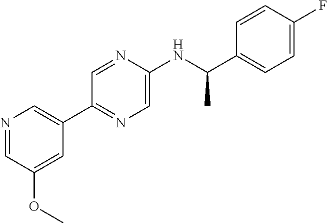 [SMILES: COc1cncc(-c2cnc(N[C@H](C)c3ccc(F)cc3)cn2)c1]

Using 3-bromo-5-methoxypyridine (50 mg, 0.27 mmol) and (R)-N-(1-(4-fluorophenyl)ethyl)-5-(4,4,5,5-tetramethyl-1,3,2-dioxaborolan-2-yl)pyridin-2-amine (Intermediate 3) (136 mg, 0.4 mmol) crude product was obtained. Purification by flash column chromatography gave (R)-N-(1-(4-fluorophenyl)ethyl)-5′-methoxy-[3,3′-bipyridin]-6-amine as a white solid (19 mg, 22%). ¹H NMR (400 MHz, DMSO-d₆): δ 8.35 (q, J=5.9 Hz, 2H), 8.17 (d, J=2.7 Hz, 1H), 7.77 (q, J=3.7 Hz, 1H), 7.50 (t, J=2.2 Hz, 1H), 7.41 (q, J=4.7 Hz, 2H), 7.29 (d, J=7.7 Hz, 1H), 7.12 (t, J=8.9 Hz, 2H), 6.59 (d, J=8.7 Hz, 1H), 5.07 (t, J=7.1 Hz, 1H), 3.86 (s, 3H), 1.44 (d, J=6.9 Hz, 3H). MS m/z: 324.19.

### Example 7: (R)-N-(1-(4-fluorophenyl)ethyl)-5′-(methylsulfonyl) [3,3′-piperidin]-6-amine

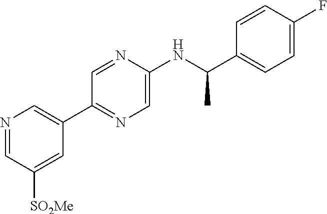 [SMILES: C[C@@H](Nc1cnc(-c2cncc(S(C)(=O)=O)c2)cn1)c1ccc(F)cc1]

Using 3-bromo-5-(methylsulfonyl)pyridine (50 mg, 0.21 mmol) and (R)-N-(1-(4-fluorophenyl)ethyl)-5-(4,4,5,5-tetramethyl-1,3,2-dioxaborolan-2-yl)pyridin-2-amine (Intermediate 3) (87 mg, 0.25 mmol), crude product was obtained. Purification by flash column chromatography gave (R)-N-(1-(4-fluorophenyl)ethyl)-5′-(methylsulfonyl)-[3,3′-bipyridin]-6-amine as a white solid (23 mg, 29%). ¹H NMR (400 MHz, DMSO-d₆): δ 9.12 (d, J=2.0 Hz, 1H), 8.90 (d, J=2.0 Hz, 1H), 8.44-8.40 (m, 2H), 7.88 (dd, J=2.40, 8.80 Hz, 1H), 7.45-7.31 (m, 3H), 7.12 (t, J=8.9 Hz, 2H), 6.63 (d, J=8.8 Hz, 1H), 5.13-5.06 (m, 1H), 3.36 (s, 3H), 1.45 (d, J=6.9 Hz, 3H). MS m/z: 372.12.

### Example 8: (R)-N-(1-(4-fluorophenyl)ethyl)-5′-(trifluoromethyl)-[3,3′-bipyridin]-6-amine

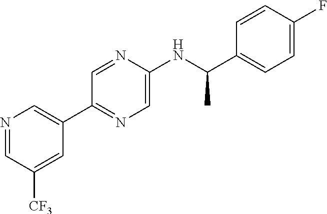 [SMILES: C[C@@H](Nc1cnc(-c2cncc(C(F)(F)F)c2)cn1)c1ccc(F)cc1]

Using 3-bromo-5-(trifluoromethyl)pyridine (50 mg, 0.22 mmol) and (R)-N-(1-(4-fluorophenyl)ethyl)-5-(4,4,5,5-tetramethyl-1,3,2-dioxaborolan-2-yl)pyridin-2-amine (Intermediate 3) (91 mg, 0.27 mmol), crude product was obtained. Purification by flash column chromatography gave (R)-N-(1-(4-fluorophenyl)ethyl)-5′-(trifluoromethyl)-[3,3′-bipyridin]-6-amine as pale yellow gum (24 mg, 30%). ¹H NMR (400 MHz, DMSO-d₆): δ 9.10 (d, J=1.7 Hz, 1H), 8.82 (s, 1H), 8.44 (d, J=2.3 Hz, 1H), 8.33 (s, 1H), 7.88 (dd, J=2.40, 8.80 Hz, 1H), 7.43-7.39 (m, 3H), 7.12 (t, J=8.9 Hz, 2H), 6.61 (d, J=8.8 Hz, 1H), 5.11-5.05 (m, 1H), 1.44 (d, J=6.9 Hz, 3H). MS m/z: 362.16.

### Example 9: (R)-5′-chloro-N-(1-(4-fluorophenyl)ethyl)-[3,3′-bipyridin]-6-amine

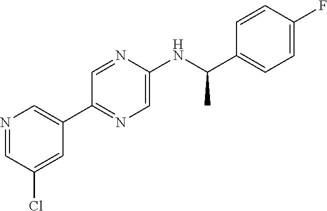 [SMILES: C[C@@H](Nc1cnc(-c2cncc(Cl)c2)cn1)c1ccc(F)cc1]

Using 3-bromo-5-chloropyridine (50 mg, 0.26 mmol) and (R)-N-(1-(4-fluorophenyl)ethyl)-5-(4,4,5,5-tetramethyl-1,3,2-dioxaborolan-2-yl)pyridin-2-amine (Intermediate 3) (107 mg, 0.31 mmol), crude product was obtained. Purification by flash column chromatography gave (R)-5′-chloro-N-(1-(4-fluorophenyl)ethyl)-[3,3′-bipyridin]-6-amine as a pale yellow gum (19 mg, 22%). ¹H NMR (400 MHz, DMSO-d₆): δ 8.76 (d, J=1.9 Hz, 1H), 8.48 (d, J=2.2 Hz, 1H), 8.37 (d, J=2.4 Hz, 1H), 8.12 (t, J=2.1 Hz, 1H), 7.81 (dd, J=2.40, 8.60 Hz, 1H), 7.43-7.39 (m, 3H), 7.12 (t, J=8.9 Hz, 2H), 6.59 (d, J=8.8 Hz, 1H), 5.12-5.04 (t, J=7.1 Hz, 1H), 1.44 (d, J=6.9 Hz, 3H). MS m/z: 328.14.

### Example 10: (R)-N-(1-(4-fluorophenyl)ethyl)-2′,5′-dimethyl-[3,3′-bipyridin]-6-amine

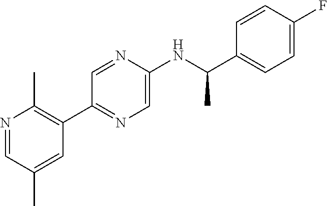 [SMILES: Cc1cnc(C)c(-c2cnc(N[C@H](C)c3ccc(F)cc3)cn2)c1]

Using 3-bromo-2,5-dimethylpyridine (75 mg, 0.4 mmol) and (R)-N-(1-(4-fluorophenyl)ethyl)-5-(4,4,5,5-tetramethyl-1,3,2-dioxaborolan-2-yl)pyridin-2-amine (Intermediate 3) (165 mg, 0.484 mmol), crude product was obtained. Purification by flash column chromatography gave (R)-N-(1-(4-fluorophenyl)ethyl)-2′,5′-dimethyl-[3,3′-bipyridin]-6-amine as a pale brown solid (34 mg, 26%). ¹H NMR (400 MHz, DMSO-d₆): δ 8.21 (d, J=1.5 Hz, 1H), 7.92 (d, J=2.2 Hz, 1H), 7.44-7.40 (m, 3H), 7.35 (d, J=1.7 Hz, 1H), 7.19 (d, J=7.7 Hz, 1H), 7.12 (t, J=8.9 Hz, 2H), 6.56 (d, J=8.6 Hz, 1H), 5.09-5.02 (m, 1H), 2.36 (s, 3H), 2.25 (s, 3H), 1.43 (d, J=6.9 Hz, 3H). MS m/z: 322.25.

### Example 11: (R)-N-(1-(4-fluorophenyl)ethyl)-4′,5′-dimethyl-[3,3′-bipyridin]-6-amine

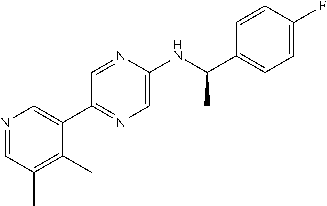 [SMILES: Cc1cncc(-c2cnc(N[C@H](C)c3ccc(F)cc3)cn2)c1C]

Using 3-bromo-2,5-dimethylpyridine (75 mg, 0.4 mmol) and (R)-N-(1-(4-fluorophenyl)ethyl)-5-(4,4,5,5-tetramethyl-1,3,2-dioxaborolan-2-yl)pyridin-2-amine (Intermediate 3) (165 mg, 0.484 mmol), crude product was obtained. Purification by flash column chromatography gave (R)-N-(1-(4-fluorophenyl)ethyl)-4′,5′-dimethyl-[3,3′-bipyridin]-6-amine as a pale brown solid (40 mg, 31%). ¹H NMR (400 MHz, DMSO-d₆): δ 8.25 (s, 1H), 8.13 (s, 1H), 7.89 (d, J=2.0 Hz, 1H), 7.45-7.37 (m, 3H), 7.21 (d, J=7.7 Hz, 1H), 7.13 (t, J=8.9 Hz, 2H), 6.57 (d, J=8.6 Hz, 1H), 5.10-5.03 (m, 1H), 2.25 (s, 3H), 2.13 (s, 3H), 1.43 (d, J=6.9 Hz, 3H). MS m/z: 322.27.

### Example 12: (R)-N6′-(1-(4-fluorophenyl)ethyl)-N5,N5-dimethyl-[3,3′-bipyridine]-5,6′-diamine

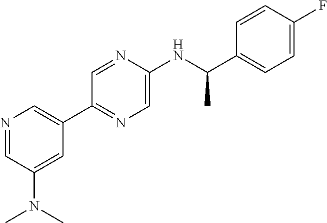 [SMILES: C[C@@H](Nc1cnc(-c2cncc(N(C)C)c2)cn1)c1ccc(F)cc1]

Using 5-bromo-N,N-dimethylpyridin-3-amine (75 mg, 0.373 mmol) and (R)-N-(1-(4-fluorophenyl)ethyl)-5-(4,4,5,5-tetramethyl-1,3,2-dioxaborolan-2-yl)pyridin-2-amine (Intermediate 3) (153 mg, 0.448 mmol) crude product was obtained. Purification by flash column chromatography gave (R)-N6′-(1-(4-fluorophenyl)ethyl)-N5,N5-dimethyl-[3,3′-bipyridine]-5,6′-diamine as an off-white solid (36 mg, 29%). ¹H NMR (400 MHz, DMSO-d₆): δ 8.29 (d, J=2.40 Hz, 1H), 8.06 (d, J=2.00 Hz, 1H), 8.00 (d, J=2.80 Hz, 1H), 7.73 (dd, J=2.80, 8.60 Hz, 1H), 7.42-7.39 (m, 2H), 7.24 (d, J=7.60 Hz, 1H), 7.16-7.08 (m, 3H), 6.58 (d, J=8.80 Hz, 1H), 5.09-5.02 (m, 1H), 2.96 (s, 6H), 1.43 (d, J=6.80 Hz, 3H). MS m/z: 337.25.

### Example 13: 5′-(ethylsulfinyl)-N-((R)-1-(4-fluorophenyl)ethyl)-[3,3′-bipyridin]-6-amine

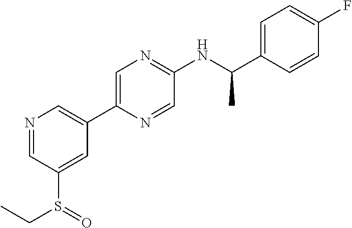 [SMILES: CCS(=O)c1cncc(-c2cnc(N[C@H](C)c3ccc(F)cc3)cn2)c1]

Using 3-bromo-5-(ethylsulfinyl)pyridine (0.1 g, 0.4 mmol) (Intermediate 5) and (R)-N-(1-(4-fluorophenyl)ethyl)-5-(4,4,5,5-tetramethyl-1,3,2-dioxaborolan-2-yl)pyridin-2-amine (Intermediate 3) (0.16 g, 0.48 mmol), crude product was obtained. Purification by flash column chromatography gave 5′-(ethylsulfinyl)-N-((R)-1-(4-fluorophenyl)ethyl)-[3,3′-bipyridin]-6-amine as an off-white solid (17 mg, 10%). ¹H NMR (400 MHz, DMSO-d₆): δ 8.94 (d, J=2.00 Hz, 1H), 8.65 (d, J=2.00 Hz, 1H), 8.39 (d, J=2.40 Hz, 1H), 8.14 (t, J=2.00 Hz, 1H), 7.84-7.81 (m, 1H), 7.43-7.38 (m, 3H), 7.12 (t, J=8.80 Hz, 2H), 6.61 (d, J=8.40 Hz, 1H), 5.10-5.07 (m, 1H), 3.19-3.13 (m, 1H), 2.95-2.90 (m, 1H), 1.44 (d, J=7.20 Hz, 3H), 1.07 (t, J=7.20 Hz, 3H). MS m/z: 370.38.

### Example 14: N-((R)-1-(4-fluorophenyl)ethyl)-5′-(methylsulfinyl)-[3,3′-bipyridin]-6-amine

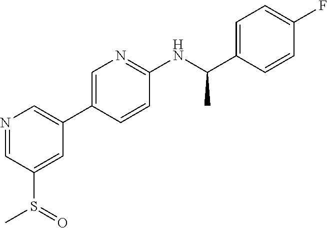 [SMILES: C[C@@H](Nc1ccc(-c2cncc(S(C)=O)c2)cn1)c1ccc(F)cc1]

Using 3-bromo-5-(methylsulfinyl)pyridine (0.16 g, 0.7 mmol) (Intermediate 4) and (R)-N-(1-(4-fluorophenyl)ethyl)-5-(4,4,5,5-tetramethyl-1,3,2-dioxaborolan-2-yl)pyridin-2-amine (Intermediate 3) (0.28 g, 0.8 mmol), crude product was obtained. Purification by column chromatography gave N-((R)-1-(4-fluorophenyl)ethyl)-5′-(methylsulfinyl)-[3,3′-bipyridin]-6-amine as pale brown solid (248 mg, 14%). ¹H NMR (400 MHz, DMSO-d₆): δ 8.94 (d, J=2.40 Hz, 1H), 8.70 (d, J=2.00 Hz, 1H), 8.40 (d, J=2.40 Hz, 1H), 8.20 (t, J=2.00 Hz, 1H), 7.84-7.82 (m, 1H), 7.43-7.39 (m, 3H), 7.12 (t, J=8.80 Hz, 2H), 6.62 (d, J=8.80 Hz, 1H), 5.12-5.05 (m, 1H), 2.89 (s, 3H), 1.44 (d, J=6.80 Hz, 3H). MS m/z: 356.31.

### Example 15: (R)-5′-(ethylsulfonyl)-N-(1-(4-fluorophenyl)ethyl)-[3,3′-bipyridin]-6-amine

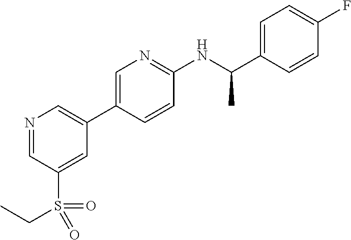 [SMILES: CCS(=O)(=O)c1cncc(-c2ccc(N[C@H](C)c3ccc(F)cc3)nc2)c1]

Using 3-bromo-5-(ethylsulfonyl)pyridine (0.1 g, 0.4 mmol) (Intermediate 6) and (R)-N-(1-(4-fluorophenyl)ethyl)-5-(4,4,5,5-tetramethyl-1,3,2-dioxaborolan-2-yl)pyridin-2-amine (Intermediate 3) (0.16 g, 0.48 mmol), crude product was obtained. Purification by flash column chromatography gave (R)-5′-(ethylsulfonyl)-N-(1-(4-fluorophenyl)ethyl)-[3,3′-bipyridin]-6-amine as an off-white solid (14 mg, 10%). ¹H NMR (400 MHz, DMSO-d₆): δ 9.14 (d, J=2.00 Hz, 1H), 8.86 (d, J=2.00 Hz, 1H), 8.44 (d, J=2.00 Hz, 1H), 8.35 (s, 1H), 7.87 (dd, J=2.80, 8.80 Hz, 1H), 7.45-7.39 (m, 3H), 7.12 (t, J=8.80 Hz, 2H), 6.63 (d, J=8.80 Hz, 1H), 5.10-5.07 (m, 1H), 3.45 (q, J=7.60 Hz, 2H), 1.45 (d, J=6.80 Hz, 3H), 1.14 (t, J=7.20 Hz, 3H). MS m/z: 386.35.

### Example 16: 1-(6′-(((R)-1-(4-fluorophenyl)ethyl)amino)-[3,3′-bipyridin]-5-yl)ethan-1-ol

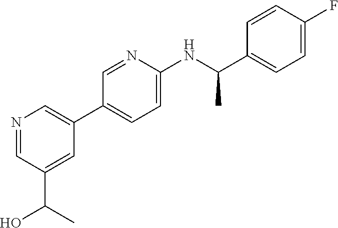 [SMILES: CC(O)c1cncc(-c2ccc(N[C@H](C)c3ccc(F)cc3)nc2)c1]

Using 1-(5-bromopyridin-3-yl)ethan-1-ol (147 mg, 0.7 mmol) (Intermediate 7) and (R)-N-(1-(4-fluorophenyl)ethyl)-5-(4,4,5,5-tetramethyl-1,3,2-dioxaborolan-2-yl)pyridin-2-amine (Intermediate 3) (263 mg, 0.77 mmol), crude product was obtained. Purification by flash column chromatography gave 1-(6′-(((R)-1-(4-fluorophenyl)ethyl)amino)-[3,3′-bipyridin]-5-yl)ethan-1-ol as a mixture of diastereomers. The diastereomers were separated by preparative SFC (Chiralcel OX-H (30*250) mm, 5 μm; 80% CO₂, 20% IPA; total flow: 100 g/min; back pressure: 100 bar) to give 16a (Isomer 1) (22 mg, 28%) and 16b (Isomer 2) (25 mg, 28%) as off-white solids.

Example 16a (Isomer 1): ¹H NMR (400 MHz, DMSO-d₆): δ 8.63 (d, J=2.40 Hz, 1H), 8.43 (d, J=1.60 Hz, 1H), 8.29 (d, J=2.40 Hz, 1H), 7.84 (s, 1H), 7.73 (dd, J=2.40, 8.80 Hz, 1H), 7.43-7.40 (m, 2H), 7.30 (d, J=15.20 Hz, 1H), 7.12 (t, J=8.80 Hz, 2H), 6.60 (d, J=8.80 Hz, 1H), 5.31 (d, J=4.40 Hz, 1H), 5.09-5.05 (m, 1H), 4.82-4.77 (m, 1H), 1.44 (d, J=6.80 Hz, 3H), 1.38 (d, J=6.40 Hz, 3H). MS m/z: 338.29. Analyt. SFC: 99.3% (5.19 min).

Example 16b (Isomer 2): ¹H NMR (400 MHz, DMSO-d₆): δ 8.63 (d, J=2.40 Hz, 1H), 8.43 (d, J=1.60 Hz, 1H), 8.29 (d, J=2.40 Hz, 1H), 7.84 (s, 1H), 7.73 (dd, J=2.40, 8.80 Hz, 1H), 7.43-7.40 (m, 2H), 7.30 (d, J=15.20 Hz, 1H), 7.12 (t, J=8.80 Hz, 2H), 6.60 (d, J=8.80 Hz, 1H), 5.31 (d, J=4.40 Hz, 1H), 5.09-5.05 (m, 1H), 4.82-4.77 (m, 1H), 1.44 (d, J=6.80 Hz, 3H), 1.38 (d, J=6.40 Hz, 3H). MS m/z: 338.29. Analyt. SFC: 97.7% (6.51 min).

### Example 17: (R)-5-(5-ethylpyridin-3-yl)-N-(1-(4-fluorophenyl)ethyl)pyrazin-2-amine

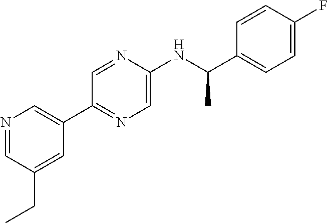 [SMILES: CCc1cncc(-c2cnc(N[C@H](C)c3ccc(F)cc3)cn2)c1]

Using 3-bromo-5-ethylpyridine (60 mg, 0.3 mmol) and (R)-(5-((1-(4-fluorophenyl)ethyl)amino)pyrazin-2-yl)boronic acid (Intermediate 8) (110 mg, 0.3 mmol), crude product was obtained. Purification by flash column chromatography gave (R)-5-(5-ethylpyridin-3-yl)-N-(1-(4-fluorophenyl)ethyl)pyrazin-2-amine as a pale brown solid (20 mg, 18%). ¹HNMR (400 MHz, DMSO-d₆): δ 8.90 (s, 1H), 8.57 (s, 1H), 8.36 (s, 1H), 8.08 (s, 1H), 7.83 (d, J=7.5 Hz, 2H), 7.44-7.40 (m, 1H), 7.13 (t, J=8.8 Hz, 2H), 5.08-5.05 (m, 1H), 2.66 (q, J=7.6 Hz, 2H), 1.47 (d, J=6.9 Hz, 3H), 1.22 (t, J=7.6 Hz, 3H). MS m/z: 323.19.

### Example 18: (R)-N-(1-(4-fluorophenyl)ethyl)-5-(5-(methylsulfonyl)pyridin-3-yl)pyrazin-2-amine

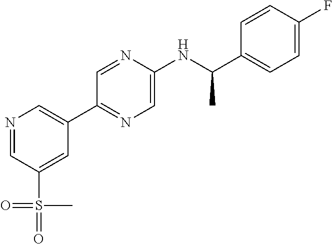 [SMILES: C[C@@H](Nc1cnc(-c2cncc(S(C)(=O)=O)c2)cn1)c1ccc(F)cc1]

Using bromo-5-(methylsulfonyl)pyridine (103 mg, 0.44 mmol) and (R)-(5-((1-(4-fluorophenyl)ethyl)amino)pyrazin-2-yl)boronic acid (Intermediate 8) (200 mg, 0.58 mmol), crude product was obtained. Purification by flash column chromatography gave (R)-N-(1-(4-fluorophenyl)ethyl)-5-(5-(methylsulfonyl)pyridin-3-yl)pyrazin-2-amine as a pale yellow solid (28 mg, 13%). ¹HNMR (400 MHz, DMSO-d₆): δ 9.40 (d, J=1.60 Hz, 1H), 8.97 (d, J=2.00 Hz, 1H), 8.74 (s, 1H), 8.69 (s, 1H), 8.13 (s, 1H), 8.04 (d, J=7.20 Hz, 1H), 7.44-7.41 (m, 2H), 7.14 (t, J=8.80 Hz, 2H), 5.13-5.06 (m, 1H), 3.36 (s, 3H), 1.48 (d, J=6.80 Hz, 3H). MS m/z: 373.16.

### Example 19: (R)-5′-ethyl-N-(1-(2-fluorophenyl)ethyl)-[3,3′-bipyridin]-6-amine

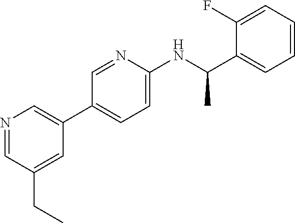 [SMILES: CCc1cncc(-c2ccc(N[C@H](C)c3ccccc3F)nc2)c1]

Using 3-bromo-5-ethylpyridine (0.075 g, 0.40 mmol) and (R)-N-(1-(2-fluorophenyl)ethyl)-5-(4,4,5,5-tetramethyl-1,3,2-dioxaborolan-2-yl)pyridin-2-amine (Intermediate 9) (0.206 g, 0.61 mmol), crude product was obtained. Purification by flash column chromatography gave (R)-5′-ethyl-N-(1-(2-fluorophenyl)ethyl)-[3,3′-bipyridin]-6-amine (18 mg, 14%) as a yellow gum. ¹H NMR (400 MHz, DMSO-d₆): δ 8.58 (d, J=1.8 Hz, 1H), 8.31-8.29 (m, 2H), 7.78-7.74 (m, 2H), 7.42 (t, J=7.7 Hz, 1H), 7.33 (d, J=7.7 Hz, 1H), 7.27-7.22 (m, 1H), 7.14-7.11 (m, 2H), 6.64 (d, J=8.7 Hz, 1H), 5.38-5.31 (m, 1H), 2.63 (d, J=7.6 Hz, 2H), 1.45 (d, J=6.9 Hz, 3H), 1.21 (t, J=7.6 Hz, 3H). MS m/z: 322.28.

### Example 20: (R)-N-(1-(4-fluorophenyl)ethyl)-5′-vinyl-[3,3′-bipyridin]-6-amine

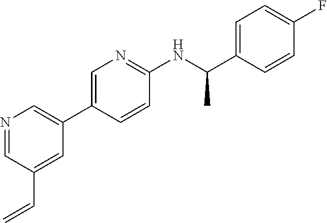 [SMILES: C=Cc1cncc(-c2ccc(N[C@H](C)c3ccc(F)cc3)nc2)c1]

Using (R)-5′-bromo-N-(1-(4-fluorophenyl)ethyl)-[3,3′-bipyridin]-6-amine (Intermediate 11) (0.15 g, 0.40 mmol) and potassium trifluoro(vinyl)borate (0.064 g, 0.48 mmol), crude product was obtained. Purification by flash column chromatography gave (R)-N-(1-(4-fluorophenyl)ethyl)-5′-vinyl-[3,3′-bipyridin]-6-amine as a yellow gummy solid (40 mg, 31%). ¹H NMR (400 MHz, DMSO-d₆): δ 8.67 (s, 1H), 8.53 (s, 1H), 8.36 (d, J=1.8 Hz, 1H), 8.07 (s, 1H), 7.80-7.78 (m, 1H), 7.43-7.39 (m, 2H), 7.30 (d, J=7.7 Hz, 1H), 7.12 (t, J=8.7 Hz, 2H), 6.78 (q, J=9.6 Hz, 1H), 6.60 (d, J=8.7 Hz, 1H), 6.09 (d, J=17.7 Hz, 1H), 5.42 (d, J=11.1 Hz, 1H), 5.09-5.04 (m, 1H), 1.44 (d, J=6.9 Hz, 3H). MS m/z: 320.19.

### Example 21: (R)-N-(2,3-dihydro-1H-inden-1-yl)-5′-ethyl-[3,3′-bipyridin]-6-amine

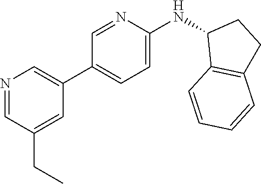 [SMILES: CCc1cncc(-c2ccc(N[C@@H]3CCc4ccccc43)nc2)c1]

Using (R)-5-bromo-N-(2,3-dihydro-1H-inden-1-yl)pyridin-2-amine (Intermediate 14) (0.1 g, 0.34 mmol) and 3-ethyl-5-(4,4,5,5-tetramethyl-1,3,2-dioxaborolan-2-yl)pyridine (Intermediate 13 (0.145 g, 0.622 mmol), crude product was obtained. Purification by flash column chromatography gave (R)-N-(2,3-dihydro-1H-inden-1-yl)-5′-ethyl-[3,3′-bipyridin]-6-amine (100 mg, 37%) as a white solid. ¹H NMR (400 MHz, DMSO-d₆): δ 8.65 (d, J=1.8 Hz, 1H), 8.41 (d, J=2.2 Hz, 1H), 8.35 (s, 1H), 7.84 (s, 1H), 7.80-7.77 (m, 1H), 7.27-7.12 (m, 5H), 6.65 (d, J=8.7 Hz, 1H), 5.60-5.57 (m, 1H), 3.00-2.93 (m, 1H), 2.87-2.79 (m, 1H), 2.69-2.66 (m, 2H), 2.51-2.50 (m, 1H), 1.88-1.82 (m, 1H), 1.25 (t, J=7.6 Hz, 3H). MS m/z: 316.24.

### Example 22: (R)-N-(1-(4-fluorophenyl)ethyl)-2-methyl-5′-(methylsulfonyl)-[3,3′-bipyridin]-6-amine

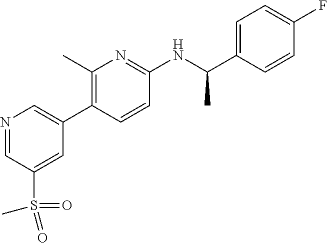 [SMILES: Cc1nc(N[C@H](C)c2ccc(F)cc2)ccc1-c1cncc(S(C)(=O)=O)c1]

Using (R)-N-(1-(4-fluorophenyl)ethyl)-6-methyl-5-(4,4,5,5-tetramethyl-1,3,2-dioxaborolan-2-yl)pyridin-2-amine (Intermediate 15) (0.23 g, 0.64 mmol) and 3-bromo-5-(methylsulfonyl)pyridine (0.18 g, 0.77 mmol), crude product was obtained. Purification by flash column chromatography gave (R)-N-(1-(4-fluorophenyl)ethyl)-2-methyl-5′-(methylsulfonyl)-[3,3′-bipyridin]-6-amine (20 mg, 11%) as an off-white solid. ¹H NMR (400 MHz, DMSO-d₆): δ 8.96 (d, J=2.1 Hz, 1H), 8.85 (d, J=2.0 Hz, 1H), 8.19 (t, J=2.1 Hz, 1H), 7.46-7.42 (m, 2H), 7.38 (d, J=8.5 Hz, 1H), 7.24 (d, J=7.8 Hz, 1H), 7.13 (t, J=8.9 Hz, 2H), 6.40 (d, J=8.5 Hz, 1H), 5.04 (t, J=6.8 Hz, 1H), 3.36 (s, 3H), 2.27 (s, 3H), 1.44 (d, J=6.9 Hz, 3H). MS m/z: 386.35.

### Example 23: N-((R)-1-(4-fluorophenyl)ethyl)-5-(5-(methylsulfinyl)pyridin-3-yl)pyrazin-2-amine

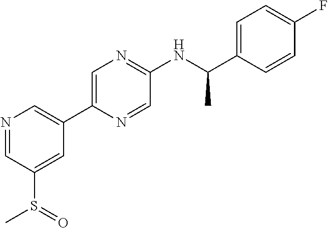 [SMILES: C[C@@H](Nc1cnc(-c2cncc(S(C)=O)c2)cn1)c1ccc(F)cc1]

Using 3-bromo-5-(methylsulfinyl)pyridine (0.1 g, 0.45 mmol) (Intermediate 4) and (R)-(5-((1-(4-fluorophenyl)ethyl)amino)pyrazin-2-yl)boronic acid (Intermediate 8) (0.45 g, 1.31 mmol), crude product was obtained. Purification by flash column chromatography gave N-((R)-1-(4-fluorophenyl)ethyl)-5-(5-(methylsulfinyl)pyridin-3-yl)pyrazin-2-amine as an off-white solid (32 mg, 20%). ¹H NMR (400 MHz, DMSO-d₆): δ 9.23 (d, J=1.8 Hz, 1H), 8.74 (d, J=1.9 Hz, 1H), 8.69 (s, 1H), 8.52 (d, J=2.2 Hz, 1H), 8.12 (s, 1H), 7.98 (d, J=7.5 Hz, 1H), 7.44-7.41 (m, 2H), 7.14 (t, J=8.8 Hz, 2H), 5.13-5.06 (m, 1H), 2.88 (s, 3H), 1.44 (d, J=6.9 Hz, 3H). MS m/z: 357.24.

### Example 24: 5-(5-(ethylsulfinyl)pyridin-3-yl)-N-((R)-1-(4-fluorophenyl)ethyl)pyrazin-2-amine

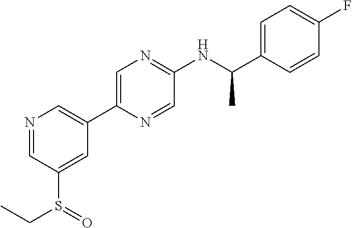 [SMILES: CCS(=O)c1cncc(-c2cnc(N[C@H](C)c3ccc(F)cc3)cn2)c1]

Using 3-bromo-5-(ethylsulfinyl)pyridine (Intermediate 5) (0.1 g, 0.43 mmol) and (R)-(5-((1-(4-fluorophenyl)ethyl)amino)pyrazin-2-yl)boronic acid (Intermediate 8) (0.425 g, 1.24 mmol), crude product was obtained. The crude was purified by flash column chromatography to furnish 5-(5-(ethylsulfinyl)pyridin-3-yl)-N-((R)-1-(4-fluorophenyl)ethyl)pyrazin-2-amine as off-white solid (28 mg, 18%). ¹H NMR (400 MHz, DMSO-d₆): δ 9.22 (d, J=1.8 Hz, 1H), 8.69-8.68 (m, 2H), 8.47 (d, J=1.5 Hz, 1H), 8.12 (s, 1H), 7.98 (d, J=7.5 Hz, 1H), 7.44-7.40 (m, 2H), 7.14 (t, J=8.8 Hz, 2H), 5.12-5.05 (m, 1H), 3.20-3.11 (m, 1H), 2.95-2.88 (m, 1H), 1.48 (d, J=6.9 Hz, 3H), 1.06 (m, J=3.6 Hz, 3H). MS m/z: 371.08.

### Example 25: (R)-N-(1-(4-fluorophenyl)ethyl)-5-(5-(methylsulfonyl)pyridin-3-yl)pyrimidin-2-amine

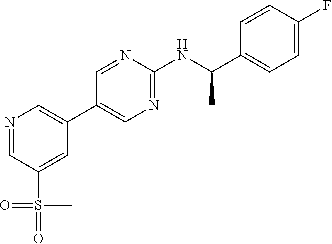 [SMILES: C[C@@H](Nc1ncc(-c2cncc(S(C)(=O)=O)c2)cn1)c1ccc(F)cc1]

Using 3-bromo-5-(methylsulfonyl)pyridine (103 mg, 0.437 mmol) and (R)-N-(1-(4-fluorophenyl)ethyl)-5-(4,4,5,5-tetramethyl-1,3,2-dioxaborolan-2-yl)pyrimidin-2-amine (Intermediate 36) (200 mg, 0.58 mmol), crude product was obtained. Purification flash column chromatography gave (R)-N-(1-(4-fluorophenyl)ethyl)-5-(5-(methylsulfonyl)pyridin-3-yl)pyrimidin-2-amine (32 mg, 20%) as an off-white solid. ¹H NMR (400 MHz, DMSO-d₆): δ 9.17 (s, 1H), 8.95 (s, 1H), 8.78 (s, 2H), 8.51 (s, 1H), 8.18 (d, J=8.00 Hz, 1H), 7.45-7.42 (m, 2H), 7.14-7.10 (m, 2H), 5.18-5.14 (m, 1H), 3.37 (s, 3H), 1.46 (d, J=6.80 Hz, 3H). MS m/z: 373.22.

### Example 26: (R)-6-(5-((1-(4-fluorophenyl)ethyl)amino)pyrazin-2-yl)-1-methyloxazolo[5,4-b]pyridin-2(1H)-one

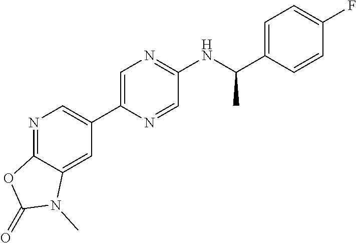 [SMILES: C[C@@H](Nc1cnc(-c2cnc3oc(=O)n(C)c3c2)cn1)c1ccc(F)cc1]

Using 6-bromo-1-methyloxazolo[5,4-b]pyridin-2(1H)-one (50 mg, 0.218 mmol) and (R)-(5-((1-(4-fluorophenyl)ethyl)amino)pyrazin-2-yl)boronic acid (Intermediate 8) (86 mg, 0.33 mmol), crude product was obtained. Purification by flash column chromatography gave (R)-6-(5-((1-(4-fluorophenyl)ethyl)amino)pyrazin-2-yl)-1-methyloxazolo[5,4-b]pyridin-2(1H)-one (9 mg, 11%) as a pale green solid. ¹H NMR (400 MHz, DMSO-d₆): δ 8.58 (d, J=0.80 Hz, 1H), 8.49 (d, J=1.60 Hz, 1H), 8.09-8.07 (m, 2H), 7.84 (d, J=7.60 Hz, 1H), 7.44-7.40 (m, 2H), 7.14 (t, J=8.80 Hz, 2H), 5.08-5.03 (m, 1H), 3.38 (s, 3H), 1.47 (d, J=6.80 Hz, 3H). MS m/z: 366.21.

### Example 27: (6′-(((R)-1-(4-fluorophenyl)ethyl)amino)-[3,3′-bipyridin]-5-yl)(imino)(methyl)-λ⁶-sulfanone

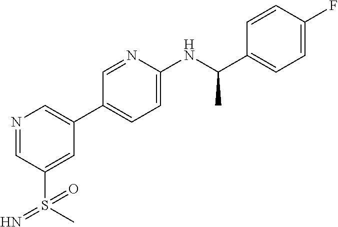 [SMILES: C[C@@H](Nc1ccc(-c2cncc(S(C)(=N)=O)c2)cn1)c1ccc(F)cc1]

Using (5-bromopyridin-3-yl)(imino)(methyl)-λ⁶-sulfanone (Intermediate 16) (100 mg, 0.4 mmol) and (R)-N-(1-(4-fluorophenyl)ethyl)-5-(4,4,5,5-tetramethyl-1,3,2-dioxaborolan-2-yl)pyridin-2-amine (Intermediate 3) (150 mg, 0.44 mmol), crude product was obtained. Purification by flash column chromatography gave (6′-(((R)-1-(4-fluorophenyl)ethyl)amino)-[3,3′-bipyridin]-5-yl)(imino)(methyl)-λ6-sulfanone as a pale brown solid (10 mg, 6%). The isomers were separated by SFC to afford Example 27a (Isomer 1) and Example 27b (Isomer 2).

Preparative SFC Conditions: Column/dimensions: Chiralcel OJ-H (30×250) mm, 5 μm; % CO₂: 75%; % Co-solvent: 25% (methanol). Total Flow: 100 g/min; Back Pressure: 100 bar

Example 27: ¹H NMR (400 MHz, DMSO-d₆): δ 9.03 (d, J=1.60 Hz, 1H), 8.90 (d, J=2.00 Hz, 1H), 8.41 (d, J=2.00 Hz, 1H), 8.35 (s, 1H), 7.85 (dd, J=2.40, 8.80 Hz, 1H), 7.43-7.40 (m, 3H), 7.12 (t, J=8.80 Hz, 2H), 6.63 (d, J=8.40 Hz, 1H), 5.11-5.04 (m, 1H), 4.51 (s, 1H), 3.10 (s, 3H), 1.44 (d, J=7.6 Hz, 3H). MS m/z: 371.25.

Example 27a (Isomer 1): ¹H NMR (400 MHz, DMSO-d₆): δ 9.03 (d, J=1.60 Hz, 1H), 8.90 (d, J=2.00 Hz, 1H), 8.41 (d, J=2.00 Hz, 1H), 8.35-8.34 (m, 1H), 7.85 (dd, J=2.40, 8.80 Hz, 1H), 7.43-7.40 (m, 3H), 7.12 (t, J=8.80 Hz, 2H), 6.63 (d, J=8.40 Hz, 1H), 5.12-5.07 (m, 1H), 4.51 (s, 1H), 3.19 (s, 3H), 1.44 (d, J=7.6 Hz, 3H). MS m/z: 371.25. Analyt. chiral SFC: 99.9% (2.30 min).

Example 27b (Isomer 2): ¹H NMR (400 MHz, DMSO-d₆): δ 9.03 (d, J=1.60 Hz, 1H), 8.90 (d, J=2.00 Hz, 1H), 8.41 (d, J=2.00 Hz, 1H), 8.35-8.34 (m, 1H), 7.85 (dd, J=2.40, 8.80 Hz, 1H), 7.43-7.40 (m, 3H), 7.12 (t, J=8.80 Hz, 2H), 6.63 (d, J=8.40 Hz, 1H), 5.12-5.07 (m, 1H), 4.51 (s, 1H), 3.19 (s, 3H), 1.44 (d, J=7.6 Hz, 3H). MS m/z: 371.25. Analyt. chiral SFC: 99.7% (2.99 min).

### Example 28: (R)-N-(1-(4-fluorophenyl)ethyl)-6-(5-(methylsulfonyl)pyridin-3-yl)-1,2,4-triazin-3-amine

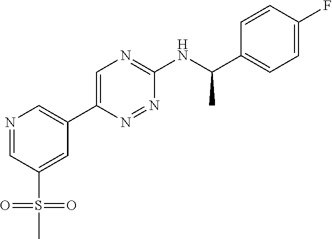 [SMILES: C[C@@H](Nc1ncc(-c2cncc(S(C)(=O)=O)c2)nn1)c1ccc(F)cc1]

Using (R)-6-chloro-N-(1-(4-fluorophenyl)ethyl)-1,2,4-triazin-3-amine (Intermediate 38) (150 mg, 0.595 mmol) and 3-(methylsulfonyl)-5-(4,4,5,5-tetramethyl-1,3,2-dioxaborolan-2-yl)pyridine (202 mg, 0.714 mmol), crude product was obtained. Purification by flash column chromatography gave (R)-N-(1-(4-fluorophenyl)ethyl)-6-(5-(methylsulfonyl)pyridin-3-yl)-1,2,4-triazin-3-amine (40 mg, 18%) as a pale yellow solid. ¹HNMR (400 MHz, DMSO-d₆): δ 9.46 (d, J=1.6 Hz, 1H), 9.09-8.61 (m, 4H), 7.52-7.45 (m, 2H), 7.15 (t, J=8.8 Hz, 2H), 5.23 (br s, 1H), 3.39 (s, 3H), 1.52 (d, J=7.0 Hz, 3H). MS m/z: 374.05.

### Example 29: 4-fluoro-N-(1-(4-fluorophenyl)ethyl)-5′-(methylsulfonyl)-[3,3′-bipyridin]-6-amine

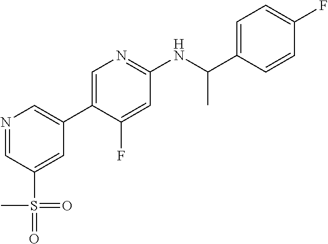 [SMILES: CC(Nc1cc(F)c(-c2cncc(S(C)(=O)=O)c2)cn1)c1ccc(F)cc1]

Using 5-bromo-4-fluoro-N-(1-(4-fluorophenyl)ethyl)pyridin-2-amine (Intermediate 18) (0.28 g, 0.89 mmol) and 3-(methylsulfonyl)-5-(4,4,5,5-tetramethyl-1,3,2-dioxaborolan-2-yl)pyridine (0.304 g, 1.07 mmol), crude product was obtained. The crude was purified by flash column chromatography to furnish the desired product as a mixture of isomers. The isomers were separated by SFC (Chiralcel OJ-H (30×250) mm, 5 μm; 80% CO₂: 20% MeOH; total flow: 100 g/min; back pressure: 100 bar) to afford Enantiomer 1 (Example 29a) (80 mg, 22%) and Enantiomer 2 (Example 29b) (60 mg, 18%) as off-white solids.

Example 29a (Enantiomer 1): ¹H NMR (400 MHz, DMSO-d₆): δ 8.99-8.97 (m, 2H), 8.35 (s, 1H), 8.28 (d, J=11.6 Hz, 1H), 7.72 (d, J=7.7 Hz, 1H), 7.44-7.40 (m, 2H), 7.14 (t, J=8.8 Hz, 2H), 6.42 (d, J=13.6 Hz, 1H), 5.08 (s, 1H), 3.36 (s, 3H), 1.45 (d, J=6.9 Hz, 3H). MS m/z: 390.21. Analyt. chiral SFC: 99.8% (2.68 min).

Example 2b (Enantiomer 2): ¹H NMR (400 MHz, DMSO-d₆): δ 8.99-8.97 (m, 2H), 8.35 (s, 1H), 8.28 (d, J=11.6 Hz, 1H), 7.72 (d, J=7.7 Hz, 1H), 7.44-7.40 (m, 2H), 7.14 (t, J=8.8 Hz, 2H), 6.42 (d, J=13.6 Hz, 1H), 5.08 (s, 1H), 3.36 (s, 3H), 1.45 (d, J=6.9 Hz, 3H). MS m/z: 390.21. Analyt. chiral SFC: 99.8% (3.40 min).

### Example 30: (6′-(((R)-1-(4-fluorophenyl)ethyl)amino)-[3,3′-bipyridin]-5-yl)(methyl)(methylimino)-λ6-sulfanone

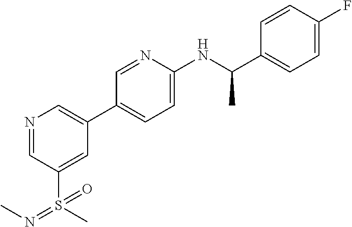 [SMILES: CN=S(C)(=O)c1cncc(-c2ccc(N[C@H](C)c3ccc(F)cc3)nc2)c1]

Using (5-bromopyridin-3-yl)(methyl)(methylimino)-λ⁶-sulfanone (Intermediate 19) (200 mg, 0.9 mmol) and (R)-N-(1-(4-fluorophenyl)ethyl)-5-(4,4,5,5-tetramethyl-1,3,2-dioxaborolan-2-yl)pyridin-2-amine (Intermediate 3) (369 mg, 1.08 mmol), crude product was obtained. Purification by flash column chromatography gave (6′-(((R)-1-(4-fluorophenyl)ethyl)amino)-[3,3′-bipyridin]-5-yl)(methyl)(methylimino)-λ6-sulfanone as a white solid (120 mg, 33%).

Example 30: ¹H NMR (400 MHz, DMSO-d₆): δ 9.06 (s, 1H), 8.80 (s, 1H), 8.42 (s, 1H), 8.24 (s, 1H), 7.86 (dd, J=2.40, 8.80 Hz, 1H), 7.43-7.40 (m, 3H), 7.12 (t, J=8.80 Hz, 2H), 6.63 (d, J=8.80 Hz, 1H), 5.11-5.07 (m, 1H), 3.25 (s, 3H), 2.50 (s, 3H), 1.44 (d, J=6.80 Hz, 3H). MS m/z: 385.27.

Diastereomers were separated by SFC (CHIRALPAK-IC-3 (30×250) mm, 5 μm; 60% CO₂, 40% MeOH; total flow: 100 g/min; back pressure: 100 bar) to give Examples 30a and 30b.

Example 30a (Isomer 1): ¹H NMR (400 MHz, DMSO-d₆): δ 9.06 (s, 1H), 8.80 (s, 1H), 8.42 (s, 1H), 8.24 (s, 1H), 7.86 (dd, J=2.40, 8.80 Hz, 1H), 7.40-7.41 (m, 3H), 7.12 (t, J=8.80 Hz, 2H), 6.63 (d, J=8.80 Hz, 1H), 5.09 (t, J=7.20 Hz, 1H), 3.25 (s, 3H), 2.50 (s, 3H), 1.44 (d, J=6.80 Hz, 3H). MS m/z: 385.27. Analyt. chiral SFC: 99.9% (2.59 min).

Example 30b (Isomer 2): ¹H NMR (400 MHz, DMSO-d₆): δ 9.06 (s, 1H), 8.80 (s, 1H), 8.42 (s, 1H), 8.24 (s, 1H), 7.86 (dd, J=2.40, 8.80 Hz, 1H), 7.40-7.41 (m, 3H), 7.12 (t, J=8.80 Hz, 2H), 6.63 (d, J=8.80 Hz, 1H), 5.09 (t, J=7.20 Hz, 1H), 3.25 (s, 3H), 2.50 (s, 3H), 1.44 (d, J=6.80 Hz, 3H). MS m/z: 385.22. Analyt. chiral SFC: 99.9% (3.46 min).

### Example 31: (R)-5-(5-(2,2-difluoroethyl)pyridin-3-yl)-N-(1-(4-fluorophenyl)ethyl)pyrazin-2-amine

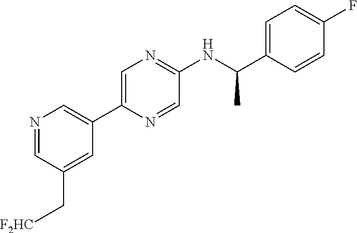

Using 3-bromo-5-(2,2-difluoroethyl)pyridine (50 mg, 0.22 mmol) and (R)-(5-((1-(4-fluorophenyl)ethyl)amino)pyrazin-2-yl)boronic acid (Intermediate 8) (89 mg, 0.33 mmol), crude product was obtained. Purification by flash column chromatography gave (R)-5-(5-(2,2-difluoroethyl)pyridin-3-yl)-N-(1-(4-fluorophenyl)ethyl)pyrazin-2-amine (15 mg, 19%) as an off-white solid. ¹H NMR (400 MHz, DMSO-d₆): δ 9.00 (s, 1H), 8.58 (s, 1H), 8.43 (s, 1H), 8.20 (s, 1H), 8.09 (s, 1H), 7.87 (d, J=7.20 Hz, 1H), 7.44-7.40 (m, 2H), 7.14 (t, J=8.80 Hz, 2H), 6.47-6.18 (m, 1H), 5.08 (t, J=6.80 Hz, 1H), 3.27-3.23 (m, 2H), 1.47 (d, J=6.80 Hz, 3H). MS m/z: 359.24.

### Example 32: 2,2,2-trifluoro-1-(5-(5-(((R)-1-(4-fluorophenyl)ethyl)amino)pyrazin-2-yl)pyridin-3-yl)ethan-1-ol

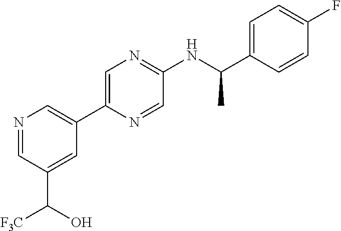 [SMILES: C[C@@H](Nc1cnc(-c2cncc(C(O)C(F)(F)F)c2)cn1)c1ccc(F)cc1]

Using 1-(5-bromopyridin-3-yl)-2,2,2-trifluoroethan-1-ol (1 g, 3.86 mmol) (Intermediate 20) and (R)-(5-((1-(4-fluorophenyl)ethyl)amino)pyrazin-2-yl)boronic acid (Intermediate 8) (1.21 g, 4.633 mmol), crude product was obtained. Purification by flash column chromatography gave 2,2,2-trifluoro-1-(5-(5-(((R)-1-(4-fluorophenyl)ethyl)amino)pyrazin-2-yl)pyridin-3-yl)ethan-1-ol (500 mg, 33%) as a mixture of diastereomers. Separation by SFC (CHIRALPAK-IG-3 (30×250) mm, 5 μm; 60% CO₂, 40% MeOH; total flow: 100 g/min, back pressure: 100 bar) allowed isolation of both diastereomers.

Example 32: ¹H NMR (400 MHz, DMSO-d₆): δ 9.09 (d, J=1.6 Hz, 1H), 8.59 (d, J=7.1 Hz, 2H), 8.36 (s, 1H), 8.10 (s, 1H), 7.91 (d, J=7.5 Hz, 1H), 7.44-7.40 (m, 2H), 7.15-7.05 (m, 3H), 5.35 (d, J=7.0 Hz, 1H), 5.08 (t, J=7.0 Hz, 1H), 1.47 (d, J=6.9 Hz, 3H). MS m/z: 393.24.

Example 32a (Isomer 1): ¹H NMR (400 MHz, DMSO-d₆): δ 9.09 (s, 1H), 8.59 (d, J=7.1 Hz, 2H), 8.36 (s, 1H), 8.10 (s, 1H), 7.91 (d, J=7.5 Hz, 1H), 7.44-7.41 (m, 2H), 7.12 (t, J=8.8 Hz, 2H), 7.06 (d, J=4.8 Hz, 1H), 5.35 (d, J=7.0 Hz, 1H), 5.10-5.06 (m, 1H), 1.47 (d, J=6.9 Hz, 3H). MS m/z: 393.25. Analyt. chiral SFC: 1.83 min.

Example 32b (Isomer 2): ¹H NMR (400 MHz, DMSO-d₆): δ 9.09 (s, 1H), 8.59 (d, J=7.1 Hz, 2H), 8.36 (s, 1H), 8.10 (s, 1H), 7.91 (d, J=7.5 Hz, 1H), 7.44-7.41 (m, 2H), 7.15-7.08 (m, 3H), 5.35 (d, J=7.0 Hz, 1H), 5.10-5.06 (m, 1H), 1.47 (d, J=6.9 Hz, 3H). MS m/z: 393.29. Analyt. chiral SFC: 2.65 min.

### Example 33: (R)-N-(1-(4-fluorophenyl)ethyl)-5-(5-vinylpyridin-3-yl)pyrazin-2-amine

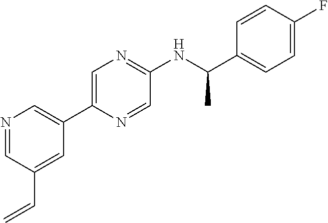 [SMILES: C=Cc1cncc(-c2cnc(N[C@H](C)c3ccc(F)cc3)cn2)c1]

Using 3-bromo-5-vinylpyridine (100 mg, 0.54 mmol) and (R)-(5-((1-(4-fluorophenyl)ethyl)amino)pyrazin-2-yl)boronic acid (Intermediate 8) (212 mg, 0.80 mmol), crude product was obtained. Purification by flash column chromatography gave (R)-N-(1-(4-fluorophenyl)ethyl)-5-(5-vinylpyridin-3-yl)pyrazin-2-amine (30 mg, 17%) as an off-white solid. ¹H NMR (400 MHz, DMSO-d₆): δ 8.98 (d, J=2.00 Hz, 1H), 8.60 (dd, J=1.60, 20.20 Hz, 2H), 8.33 (t, J=2.00 Hz, 1H), 8.09 (d, J=1.20 Hz, 1H), 7.88 (d, J=7.60 Hz, 1H), 7.44-7.40 (m, 2H), 7.14 (t, J=8.9 Hz, 1H), 6.48-6.47 (m, 1H), 6.06 (d, J=17.60 Hz, 1H), 5.43 (d, J=11.20 Hz, 1H), 5.09-5.05 (m, 1H), 1.47 (d, J=6.80 Hz, 3H). MS m/z: 321.19.

### Example 34: (R)-5-(5-(2,2-difluorovinyl)pyridin-3-yl)-N-(1-(4-fluorophenyl)ethyl)pyrazin-2-amine

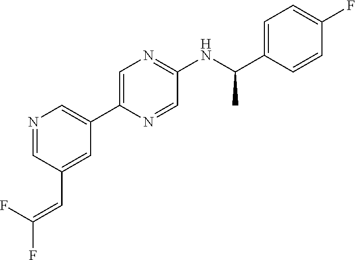 [SMILES: C[C@@H](Nc1cnc(-c2cncc(C=C(F)F)c2)cn1)c1ccc(F)cc1]

Using 3-bromo-5-(2,2-difluorovinyl)pyridine (Intermediate 21) (60 mg, 318.2 mmol) and (R)-(5-((1-(4-fluorophenyl)ethyl)amino)pyrazin-2-yl)boronic acid (Intermediate 8) (100 mg, 381.8 mmol), crude product was obtained. Purification by flash column chromatography gave (R)-5-(5-(2,2-difluorovinyl)pyridin-3-yl)-N-(1-(4-fluorophenyl)ethyl)pyrazin-2-amine (20 mg, 20%) as a white solid. ¹H NMR (400 MHz, DMSO-d₆): δ 8.97 (d, J=1.9 Hz, 1H), 8.60 (s, 1H), 8.49 (d, J=1.4 Hz, 1H), 8.24 (s, 1H), 8.10 (s, 1H), 7.91 (d, J=7.5 Hz, 1H), 7.44-7.40 (m, 2H), 7.14 (t, J=8.9 Hz, 2H), 5.96-5.88 (m, 1H), 5.11-5.04 (m, 1H), 1.47 (d, J=6.9 Hz, 3H). MS m/z: 357.24.

### Example 35: (R)-2-(5-(5-((1-(4-fluorophenyl)ethyl)amino)pyrazin-2-yl)pyridin-3-yl)ethan-1-ol

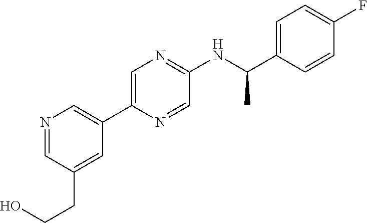 [SMILES: C[C@@H](Nc1cnc(-c2cncc(CCO)c2)cn1)c1ccc(F)cc1]

Using 2-(5-bromopyridin-3-yl)ethan-1-ol (Intermediate 22) (0.054 g, 0.3 mmol) and (R)-(5-((1-(4-fluorophenyl)ethyl)amino)pyrazin-2-yl)boronic acid (Intermediate 8) (0.1 g, 0.3 mmol), crude product was obtained. Purification by flash column chromatography gave (R)-2-(5-(5-((1-(4-fluorophenyl)ethyl)amino)pyrazin-2-yl)pyridin-3-yl)ethan-1-ol (40 mg, 40%) as a white solid. ¹H NMR (400 MHz, DMSO-d₆): δ 8.90 (d, J=2.00 Hz, 1H), 8.56 (s, 1H), 8.35 (d, J=1.60 Hz, 1H), 8.08 (s, 2H), 7.83 (d, J=7.20 Hz, 1H), 7.44-7.40 (m, 2H), 7.13 (t, J=8.80 Hz, 2H), 5.07 (t, J=7.20 Hz, 1H), 4.70 (t, J=5.20 Hz, 1H), 3.65 (q, J=6.40 Hz, 2H), 2.76 (t, J=6.80 Hz, 2H), 1.47 (d, J=6.80 Hz, 3H). MS m/z: 339.24.

### Example 36: (R)-N-(2,3-dihydro-1H-inden-1-yl)-5-(5-(methylsulfonyl)pyridin-3-yl)pyrazin-2-amine

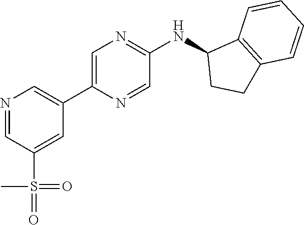 [SMILES: CS(=O)(=O)c1cncc(-c2cnc(N[C@@H]3CCc4ccccc43)cn2)c1]

Using (R)-5-chloro-N-(2,3-dihydro-1H-inden-1-yl)pyrazin-2-amine (Intermediate 39) (1.70 g, 0.694 mmol) and (5-(methylsulfonyl)pyridin-3-yl)boronic acid (Intermediate 24) (209 mg, 1.04 mmol), crude product was obtained. Purification by column chromatography gave (R)-N-(2,3-dihydro-1H-inden-1-yl)-5-(5-(methylsulfonyl)pyridin-3-yl)pyrazin-2-amine (142 mg, 56%) as an off-white solid. ¹H NMR (400 MHz, DMSO-d₆): δ 9.47 (d, J=1.9 Hz, 1H), 8.99 (d, J=2.0 Hz, 1H), 8.86 (s, 1H), 8.74-8.73 (m, 1H), 8.14 (s, 1H), 7.94 (d, J=7.9 Hz, 1H), 7.30-7.16 (m, 4H), 5.56 (q, J=7.5 Hz, 1H), 3.39 (s, 3H), 3.04-3.00 (m, 1H), 2.99-2.83 (m, 1H), 2.58-2.53 (m, 1H), 1.90-1.88 (m, 1H). MS m/z: 367.15.

### Example 37: (R)-N-(1-(4-fluorophenyl)ethyl)-5-(5-(methylamino)methylpyridin-3-yl)pyrazin-2-amine

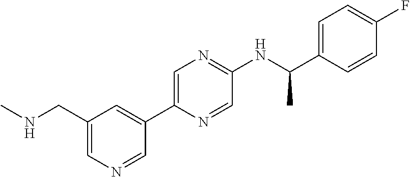 [SMILES: CNCc1cncc(-c2cnc(N[C@H](C)c3ccc(F)cc3)cn2)c1]

Using (R)-(5-((1-(4-fluorophenyl)ethyl)amino)pyrazin-2-yl)boronic acid (Intermediate 8) (58 mg, 0.3 mmol) and 1-(5-bromopyridin-3-yl)-N-methylmethanamine (103 mg, 0.3 mmol), crude product was obtained. Purification by flash column chromatography gave (R)-N-(1-(4-fluorophenyl)ethyl)-5-(5-(methylamino)methylpyridin-3-yl)pyrazin-2-amine as an off-white solid (36 mg, 35%). ¹H NMR (400 MHz, DMSO-d₆): δ 8.97 (d, J=1.8 Hz, 1H), 8.57 (s, 1H), 8.45 (d, J=1.5 Hz, 1H), 8.21 (s, 1H), 8.09 (s, 1H), 7.85 (d, J=7.5 Hz, 1H), 7.44-7.40 (m, 2H), 7.14 (t, J=8.8 Hz, 2H), 5.11-5.08 (m, 1H), 4.81-4.10 (br, 1H), 3.78 (s, 2H), 2.33 (s, 3H), 1.47 (d, J=6.9 Hz, 3H). MS m/z: 338.19.

### Example 38: N-(5-fluoro-2,3-dihydro-1H-inden-1-yl)-5′-(methylsulfonyl)-[3,3′-bipyridin]-6-amine

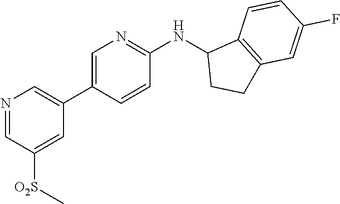 [SMILES: CS(=O)(=O)c1cncc(-c2ccc(NC3CCc4cc(F)ccc43)nc2)c1]

Using 5-bromo-N-(5-fluoro-2,3-dihydro-1H-inden-1-yl)pyridin-2-amine (Intermediate 27) (40 mg, 0.1 mmol) and 3-(methylsulfonyl)-5-(4,4,5,5-tetramethyl-1,3,2-dioxaborolan-2-yl)pyridine (34 mg, 0.12 mmol), crude product was obtained. Purification by flash column chromatography gave N-(5-fluoro-2,3-dihydro-1H-inden-1-yl)-5′-(methylsulfonyl)-[3,3′-bipyridin]-6-amine as an off-white solid (10 mg, 20%). ¹H NMR (400 MHz, DMSO-d₆): δ 9.19 (d, J=1.4 Hz, 1H), 8.93 (d, J=1.4 Hz, 1H), 8.56 (d, J=1.7 Hz, 1H), 8.46 (s, 1H), 7.93 (q, J=3.6 Hz, 1H), 7.33-7.25 (m, 2H), 7.10 (d, J=8.9 Hz, 1H), 6.98 (t, J=8.8 Hz, 1H), 6.68 (d, J=8.7 Hz, 1H), 5.58-5.52 (m, 1H), 3.39 (s, 3H), 3.01-2.95 (m, 1H), 2.89-2.83 (m, 1H), 2.56-2.55 (m, 1H), 1.92-1.87 (m, 1H). MS m/z: 384.26.

### Example 39: (R)-5-(5-(difluoromethoxy)pyridin-3-yl)-N-(1-(4-fluorophenyl)ethyl)pyrazin-2-amine

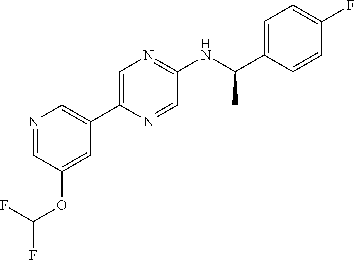 [SMILES: C[C@@H](Nc1cnc(-c2cncc(OC(F)F)c2)cn1)c1ccc(F)cc1]

Using 3-bromo-5-(difluoromethoxy)pyridine (Intermediate 28) (0.10 g, 0.4 mmol) and (R)-(5-((1-(4-fluorophenyl)ethyl)amino)pyrazin-2-yl)boronic acid (Intermediate 8) (0.114 g, 0.52 mmol), crude product was obtained. Purification by flash column chromatography gave (R)-5-(5-(difluoromethoxy)pyridin-3-yl)-N-(1-(4-fluorophenyl)ethyl)pyrazin-2-amine (80 mg, 52%) as a white solid. ¹H NMR (400 MHz, DMSO-d₆): δ 9.00 (d, J=1.5 Hz, 1H), 8.65 (s, 1H), 8.40 (d, J=2.5 Hz, 1H), 8.08 (d, J=13.8 Hz, 2H), 7.97 (d, J=7.5 Hz, 1H), 7.56-7.38 (m, 3H), 7.14 (t, J=8.8 Hz, 2H), 5.08 (t, J=7.0 Hz, 1H), 1.47 (d, J=6.9 Hz, 3H). MS m/z: 361.09.

### Example 40: (R)-6-(5-(2,2-difluorovinyl)pyridin-3-yl)-N-(1-(4-fluorophenyl)ethyl)-1,2,4-triazin-3-amine

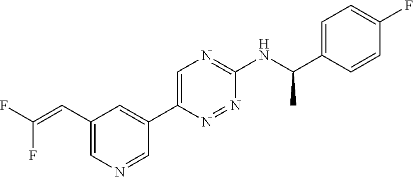 [SMILES: C[C@@H](Nc1ncc(-c2cncc(C=C(F)F)c2)nn1)c1ccc(F)cc1]

Using (R)-6-chloro-N-(1-(4-fluorophenyl)ethyl)-1,2,4-triazin-3-amine (Intermediate 38) (160 mg, 0.6 mmol) and 3-(2,2-difluorovinyl)-5-(4,4,5,5-tetramethyl-1,3,2-dioxaborolan-2-yl)pyridine (Intermediate 29) (160 mg, 0.6 mmol), crude product was obtained. Purification by flash column chromatography gave (R)-6-(5-(2,2-difluorovinyl)pyridin-3-yl)-N-(1-(4-fluorophenyl)ethyl)-1,2,4-triazin-3-amine as an off-white solid. (20 mg, 16%). ¹H NMR (400 MHz, DMSO-d₆): δ 9.03 (d, J=1.6 Hz, 1H), 8.90 (s, 1H), 8.61 (d, J=1.3 Hz, 2H), 8.36 (s, 1H), 7.48-7.44 (m, 2H), 7.14 (t, J=8.9 Hz, 2H), 6.00-5.92 (m, 1H), 5.20 (s, 1H), 1.51 (d, J=7.0 Hz, 3H). MS m/z: 358.21.

### Example 41: (R)-(5-(3-((1-(4-fluorophenyl)ethyl)amino)-1,2,4-triazin-6-yl)pyridin-3-yl)methanol

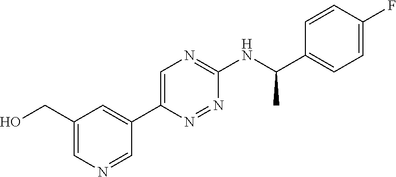 [SMILES: C[C@@H](Nc1ncc(-c2cncc(CO)c2)nn1)c1ccc(F)cc1]

Using (R)-6-chloro-N-(1-(4-fluorophenyl)ethyl)-1,2,4-triazin-3-amine (Intermediate 38) (300 mg, 1.97 mmol) and 5-(4,4,5,5-tetramethyl-1,3,2-dioxaborolan-2-yl)nicotinaldehyde (691 mg, 2.95 mmol), crude product was obtained as a mixture of the corresponding aldehyde and alcohol. Purification by flash column chromatography (50% EtOAc in petroleum ether) gave (R)-(5-(3-((1-(4-fluorophenyl)ethyl)amino)-1,2,4-triazin-6-yl)pyridin-3-yl)methanol as an off-white solid (35 mg, 5%). ¹H NMR (400 MHz, DMSO-d₆): δ 9.01 (d, J=1.6 Hz, 1H), 8.89 (s, 1H), 8.56 (d, J=1.2 Hz, 2H), 8.29 (s, 1H), 7.48-7.44 (m, 2H), 7.14 (t, J=8.8 Hz, 2H), 5.41 (t, J=5.7 Hz, 1H), 5.20 (s, 1H), 4.60 (d, J=5.6 Hz, 2H), 1.51 (d, J=7.0 Hz, 3H). MS m/z: 326.28.

### Example 42: (R)-N-(1-(4-fluorophenyl)ethyl)-5-(5-(2,2,2-trifluoroethoxy)pyridin-3-yl)pyrazin-2-amine

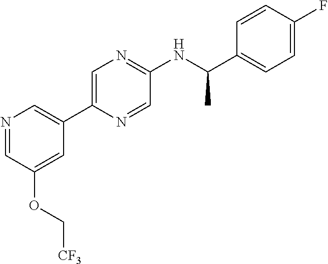 [SMILES: C[C@@H](Nc1cnc(-c2cncc(OCC(F)(F)F)c2)cn1)c1ccc(F)cc1]

Using (R)-5-chloro-N-(1-(4-fluorophenyl)ethyl)pyrazin-2-amine (Intermediate 8, step 1) (300 mg, 1.195 mmol) and 3-(4,4,5,5-tetramethyl-1,3,2-dioxaborolan-2-yl)-5-(2,2,2-trifluoroethoxy)pyridine (Intermediate 30) (396 mg, 1.793 mmol), crude product was obtained. Purification by flash column chromatography gave (R)-N-(1-(4-fluorophenyl)ethyl)-5-(5-(2,2,2-trifluoroethoxy)pyridin-3-yl)pyrazin-2-amine (165 mg, 35%) as an off-white solid. ¹HNMR (400 MHz, DMSO-d₆): δ 8.80 (s, 1H), 8.62 (s, 1H), 8.31 (d, J=2.40 Hz, 1H), 8.08 (s, 1H), 7.93-7.89 (m, 2H), 7.44-7.40 (m, 2H), 7.14 (t, J=8.80 Hz, 2H), 5.08 (t, J=6.80 Hz, 1H), 4.93 (q, J=8.80 Hz, 2H), 1.47 (d, J=6.80 Hz, 3H). MS m/z: 393.25.

### Example 43: (R)-N-(1-(4-fluorophenyl)ethyl)-5-(5-(trifluoromethoxy)pyridin-3-yl)pyrazin-2-amine

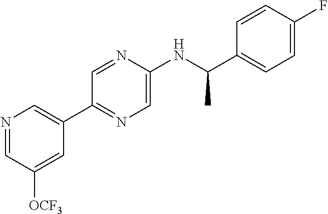 [SMILES: C[C@@H](Nc1cnc(-c2cncc(OC(F)(F)F)c2)cn1)c1ccc(F)cc1]

Using (R)-5-chloro-N-(1-(4-fluorophenyl)ethyl)pyrazin-2-amine (Intermediate 8, step 1) (78 mg, 0.3 mmol) and 3-(4,4,5,5-tetramethyl-1,3,2-dioxaborolan-2-yl)-5-(trifluoromethoxy)pyridine (Intermediate 32) (75 mg, 0.3 mmol), crude product was obtained. Purification by flash column chromatography gave (R)-N-(1-(4-fluorophenyl)ethyl)-5-(5-(trifluoromethoxy)pyridin-3-yl)pyrazin-2-amine as a white solid (20 mg, 33%). ¹H NMR (400 MHz, DMSO-d₆): δ 9.16 (s, 1H), 8.69 (s, 1H), 8.58 (s, 1H), 8.25 (s, 1H), 8.10 (s, 1H), 8.02 (d, J=7.4 Hz, 1H), 7.44-7.40 (m, 2H), 7.14 (t, J=8.8 Hz, 2H), 5.09 (t, J=7.0 Hz, 1H), 1.48 (d, J=6.9 Hz, 3H). MS m/z: 379.26.

### Example 44: (R)-N-(1-(4-fluorophenyl)ethyl)-5-(6-vinylpyridazin-4-yl)pyrazin-2-amine

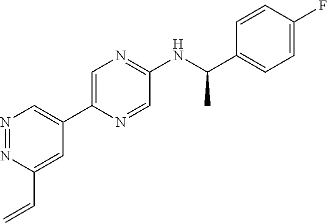 [SMILES: C=Cc1cc(-c2cnc(N[C@H](C)c3ccc(F)cc3)cn2)cnn1]

Using 5-chloro-3-vinylpyridazine (Intermediate 33) (1.5 g, 10.71 mmol) and (R)-(5-((1-(4-fluorophenyl)ethyl)amino)pyrazin-2-yl)boronic acid (Intermediate 8) (3.343 g, 12.85 mmol), crude product was obtained. Purification by flash column chromatography (30% EtOAc in petroleum ether) gave (R)-N-(1-(4-fluorophenyl)ethyl)-5-(6-vinylpyridazin-4-yl)pyrazin-2-amine (900 mg, 26%) as a pale yellow solid. ¹H NMR (400 MHz, DMSO-d₆): δ 9.61 (d, J=2.1 Hz, 1H), 8.85 (d, J=1.1 Hz, 1H), 8.26-8.20 (m, 1H), 8.14 (d, J=1.0 Hz, 1H), 7.44-7.40 (m, 2H), 7.16-7.12 (m, 2H), 7.05-6.98 (m, 1H), 6.45 (d, J=17.7 Hz, 1H), 5.72 (d, J=11.6 Hz, 1H), 5.12 (t, J=7.1 Hz, 1H), 1.49 (d, J=6.9 Hz, 3H). MS m/z: 322.23.

### Example 4s: 1-(5-(5-(((R)-2,3-dihydro-1H-inden-1-yl)amino)pyrazin-2-yl)pyridin-3-yl)-2,2-difluoroethan-1-ol

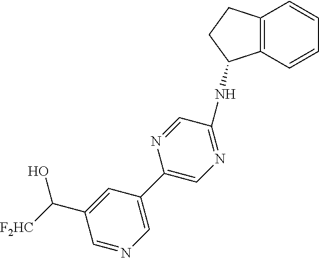 [SMILES: OC(c1cncc(-c2cnc(N[C@@H]3CCc4ccccc43)cn2)c1)C(F)F]

Using (R)-5-chloro-N-(2,3-dihydro-1H-inden-1-yl)pyrazin-2-amine (Intermediate 39) (170 g, 0.694 mmol) and 5-(4,4,5,5-tetramethyl-1,3,2-dioxaborolan-2-yl)nicotinaldehyde (242 mg, 1.04 mmol). The crude product was purified by flash column chromatography to give (R)-5-(5-((2,3-dihydro-1H-inden-1-yl)amino)pyrazin-2-yl)nicotinaldehyde (160 mg, 51%), which was used directly in the next step.

(R)-5-(5-((2,3-dihydro-1H-inden-1-yl)amino)pyrazin-2-yl)nicotinaldehyde was dissolved in THF, under N₂ atmosphere and treated with (difluoromethyl)trimethylsilane (125 mg, 1.00 mmol). The resulting mixture was cooled to 0° C., treated with TBAF (0.26 g, 1.00 mmol) and stirred at RT for 16 h. The mixture was quenched with H₂O and extracted with EtOAc. The organic layer was dried over Na₂SO₄ and concentrated under reduced pressure. The residue was purified by flash chromatography (EtOAc/petroleum ether) to give 1-(5-(5-(((R)-2,3-dihydro-1H-inden-1-yl)amino)pyrazin-2-yl)pyridin-3-yl)-2,2-difluoroethan-1-ol (19 mg, 10%) as a yellow solid. ¹H NMR (400 MHz, DMSO-d₆): δ 9.10 (d, J=2.00 Hz, 1H), 8.70 (s, 1H), 8.55 (d, J=1.20 Hz, 1H), 8.34 (s, 1H), 8.11 (s, 1H), 7.78 (d, J=8.00 Hz, 1H), 7.30-7.16 (m, 4H), 6.42 (d, J=5.20 Hz, 1H), 6.30-6.01 (m, 1H), 5.54-5.50 (m, 1H), 4.96-4.90 (m, 1H), 3.02-2.96 (m, 1H), 2.90-2.82 (m, 1H), 1.92-1.85 (m, 1H), 1.23 (s, 1H). MS m/z: 369.13.

Using the method employed in the preparation of Example 2, the following compounds were prepared:

### Example 46: N-(1-(4-chlorophenyl)ethyl)-5′-methyl-[3,3′-bipyridin]-6-amine

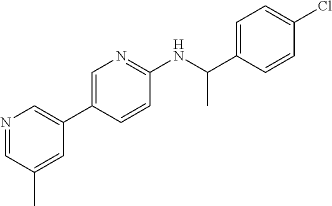 [SMILES: Cc1cncc(-c2ccc(NC(C)c3ccc(Cl)cc3)nc2)c1]

Using 6′-chloro-5-methyl-3,3′-bipyridine (Intermediate 2) (280 mg, 1.36 mmol) and 1-(4-chlorophenyl)ethan-1-amine (254 mg, 1.64 mmol), crude product was obtained. Purification by flash column chromatography (30% EtOAc in petroleum ether) furnished the racemate. Separation of enantiomers by chiral SFC (CHIRALPAK AS-3 (30×250) mm, 3 μm; 70% CO₂, 30% of 0.2% DEA in MeOH), total flow: 100 g/min; back pressure: 100 bar) gave Isomer 1 (Example 46a, 100 mg, 23%) and Isomer 2 (Example 46b, 120 mg, 27%) as off-white solids.

Example 46a: ¹H NMR (400 MHz, DMSO-d₆): δ 8.57 (d, J=2.00 Hz, 1H), 8.29-8.28 (m, 2H), 7.76-7.72 (m, 2H), 7.41 (d, J=8.40 Hz, 2H), 7.36 (d, J=8.40 Hz, 2H), 7.30 (d, J=7.20 Hz, 1H), 6.60 (d, J=8.40 Hz, 1H), 5.08-5.04 (m, 1H), 2.32 (s, 3H), 1.44 (d, J=6.80 Hz, 3H). MS m/z: 324.36. Analyt. chiral SFC: 99.6% (1.60 min).

Example 46b: ¹H NMR (400 MHz, DMSO-d₆): δ 8.57 (d, J=2.00 Hz, 1H), 8.29-8.28 (m, 2H), 7.76-7.71 (m, 2H), 7.41 (d, J=8.40 Hz, 2H), 7.36 (d, J=8.40 Hz, 2H), 7.30 (d, J=7.20 Hz, 1H), 6.60 (d, J=8.40 Hz, 1H), 5.09-5.02 (m, 1H), 2.32 (s, 3H), 1.44 (d, J=6.80 Hz, 3H). MS m/z: 324.34. Analyt. chiral SFC: 99.8% (2.08 min).

### Example 47: (R)-5′-ethyl-N-(1-(4-methoxyphenyl)ethyl)-[3,3′-bipyridin]-6-amine

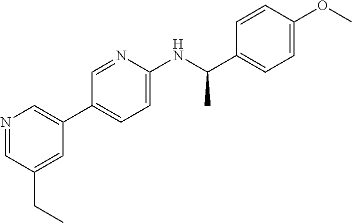 [SMILES: CCc1cncc(-c2ccc(N[C@H](C)c3ccc(OC)cc3)nc2)c1]

Using 6′-chloro-5-ethyl-3,3′-bipyridine (Intermediate 10) (0.15 g, 0.7 mmol) and (R)-1-(4-methoxyphenyl)ethan-1-amine (0.116 g, 0.77 mmol), crude product was obtained. Purification by flash column chromatography gave (R)-5′-ethyl-N-(1-(4-methoxyphenyl)ethyl)-[3,3′-bipyridin]-6-amine (50 mg, 21%) as a sticky solid. ¹H NMR (400 MHz, DMSO-d₆): δ 8.59 (d, J=1.4 Hz, 1H), 8.32-8.30 (m, 2H), 7.79 (s, 1H), 7.74-7.71 (m, 1H), 7.30 (d, J=8.5 Hz, 2H), 7.19 (d, J=7.8 Hz, 1H), 6.86 (d, J=8.5 Hz, 2H), 6.57 (d, J=8.7 Hz, 1H), 5.04-4.97 (m, 1H), 3.71 (s, 3H), 2.64 (q, J=7.6 Hz, 2H), 1.42 (d, J=6.8 Hz, 3H), 1.22 (t, J=7.6 Hz, 3H). MS m/z: 334.26.

### Example 48: (R)-5′-ethyl-N-(1-(3-fluorophenyl)ethyl)-[3,3′-bipyridin]-6-amine

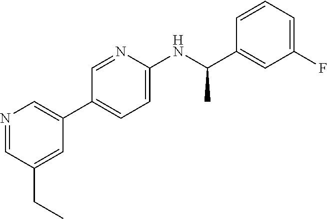 [SMILES: CCc1cncc(-c2ccc(N[C@H](C)c3cccc(F)c3)nc2)c1]

Using 6′-chloro-5-ethyl-3,3′-bipyridine (Intermediate 10) (0.15 g, 0.7 mmol) and (R)-1-(3-fluorophenyl)ethan-1-amine (0.14 g, 1.03 mmol), crude product was obtained. Purification by flash column chromatography gave (R)-5′-ethyl-N-(1-(3-fluorophenyl)ethyl)-[3,3′-bipyridin]-6-amine (45 mg, 20%) as a pale brown semi-solid. ¹H NMR (400 MHz, DMSO-d₆): δ 8.59 (d, J=2.00 Hz, 1H), 8.32-8.29 (m, 2H), 7.78-7.74 (m, 2H), 7.37-7.28 (m, 2H), 7.23-7.17 (m, 2H), 7.03-6.98 (m, 1H), 6.61 (d, J=8.40 Hz, 1H), 5.12-5.05 (m, 1H), 2.64 (q, J=7.20 Hz, 2H), 1.45 (d, J=7.20 Hz, 3H), 1.22 (t, J=7.60 Hz, 3H). MS m/z: 322.29.

### Example 49: (R)-5′-ethyl-N-(1-phenylethyl)-[3,3′-bipyridin]-6-amine

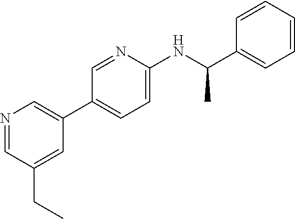 [SMILES: CCc1cncc(-c2ccc(N[C@H](C)c3ccccc3)nc2)c1]

Using 6′-chloro-5-ethyl-3,3′-bipyridine (Intermediate 10) (0.15 g, 0.7 mmol) and (R)-1-phenylethan-1-amine 0.13 g, 1.03 mmol), crude product was obtained. Purification by flash column chromatography gave (R)-5′-ethyl-N-(1-phenylethyl)-[3,3′-bipyridin]-6-amine (30 mg, 14%) as a pale yellow gummy solid. ¹H NMR (400 MHz, DMSO-d₆): δ 8.59 (s, 1H), 8.31-8.29 (m, 2H), 7.77-7.72 (m, 2H), 7.39-7.37 (m, 2H), 7.34-7.16 (m, 4H), 6.59 (d, J=8.80 Hz, 1H), 5.08-5.05 (m, 1H), 2.64 (q, J=7.60 Hz, 2H), 1.45 (d, J=6.80 Hz, 3H), 1.22 (t, J=7.60 Hz, 3H). MS m/z: 304.18.

### Example 50: (R)-5′-ethyl-N-(1-(p-tolyl)ethyl)-[3,3′-bipyridin]-6-amine

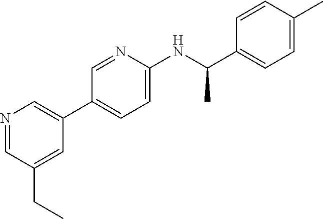 [SMILES: CCc1cncc(-c2ccc(N[C@H](C)c3ccc(C)cc3)nc2)c1]

Using 6′-chloro-5-ethyl-3,3′-bipyridine (Intermediate to) (0.15 g, 0.7 mmol) and (R)-1-(p-tolyl)ethan-1-amine (0.14 g, 1.03 mmol), crude product was obtained. Purification by flash column chromatography gave (R)-5′-ethyl-N-(1-(p-tolyl)ethyl)-[3,3′-bipyridin]-6-amine (35 mg, 16%) as a gummy solid. ¹H NMR (400 MHz, DMSO-d₆): δ 8.58 (d, J=1.60 Hz, 1H), 8.31-8.29 (m, 2H), 7.78 (s, 1H), 7.72 (dd, J=2.00, 8.80 Hz, 1H), 7.26 (d, J=8.00 Hz, 2H), 7.19 (d, J=7.60 Hz, 1H), 7.10 (d, J=7.60 Hz, 2H), 6.57 (d, J=8.80 Hz, 1H), 5.01 (t, J=7.20 Hz, 1H), 2.64 (q, J=7.60 Hz, 2H), 2.25 (s, 3H), 1.42 (d, J=6.80 Hz, 3H), 1.22 (t, J=7.60 Hz, 3H). MS m/z: 318.23.

### Example 51: (R)-5′-ethynyl-N-(1-(4-fluorophenyl)ethyl)-[3,3′-bipyridin]-6-amine

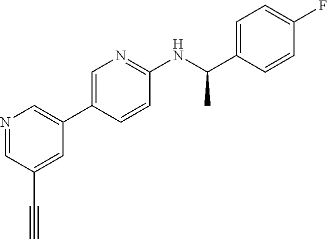 [SMILES: C#Cc1cncc(-c2ccc(N[C@H](C)c3ccc(F)cc3)nc2)c1]

A stirred solution of (R)-N-(1-(4-fluorophenyl)ethyl)-5′-((trimethylsilyl)ethynyl)-[3,3′-bipyridin]-6-amine (Intermediate 12) (800 mg, 2.1 mmol) in MeOH (20 mL) was treated with anhydrous K₂CO₃ (1.45 g, 10.5 mmol), stirred at RT for 2 h and filtered through Celite®. The filtrate was concentrated under vacuo to give the crude compound. Purification by flash chromatography (40% EtOAc in petroleum ether) gave (R)-5′-ethynyl-N-(1-(4-fluorophenyl)ethyl)-[3,3′-bipyridin]-6-amine as a white solid (80 mg, 20%). ¹H NMR (400 MHz, DMSO-d₆): δ 8.80 (d, J=2.00 Hz, 1H), 8.53 (d, J=2.00 Hz, 1H), 8.35 (d, J=2.40 Hz, 1H), 8.06 (t, J=2.00 Hz, 1H), 7.79 (dd, J=2.80, 8.80 Hz, 1H), 7.43-7.40 (m, 2H), 7.34 (d, J=7.60 Hz, 1H), 7.12 (t, J=9.20 Hz, 2H), 6.59 (d, J=8.80 Hz, 1H), 5.11-5.04 (m, 1H), 4.44 (s, 1H), 1.44 (d, J=6.80 Hz, 3H). MS m/z: 318.21.

### Example 12: (R)-5-(5-(2-fluoroethyl)pyridin-3-yl)-N-(1-(4-fluorophenyl)ethyl)pyrazin-2-amine

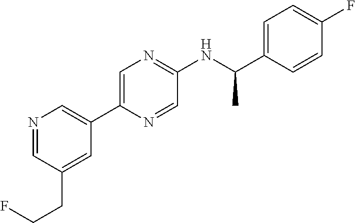 [SMILES: C[C@@H](Nc1cnc(-c2cncc(CCF)c2)cn1)c1ccc(F)cc1]

DAST (130 mg, 338.6 mmol) was added to a stirred solution of (R)-2-(5-(5-((1-(4-fluorophenyl)ethyl)amino)pyrazin-2-yl)pyridin-3-yl)ethan-1-ol (Example 35) (185 mg, 1153 mmol) in CH₂Cl₂ at −78° C. The mixture was stirred at RT for 2 h, quenched with ice H₂O and extracted with 10% MeOH/CH₂Cl₂ (2×30 mL). The combined organic layers were dried over anhydrous Na₂SO₄ and concentrated. Purification of the residue by reverse phase column chromatography (C18 column, 55% MeOH in H₂O) by SFC gave (R)-5-(5-(2-fluoroethyl)pyridin-3-yl)-N-(1-(4-fluorophenyl)ethyl)pyrazin-2-amine (15 mg, 11%) as a pale brown solid. ¹H NMR (400 MHz, DMSO-d₆): δ 8.95 (d, J=1.6 Hz, 1H), 8.57 (s, 1H), 8.41 (s, 1H), 8.15 (s, 1H), 8.08 (s, 1H), 7.85 (d, J=7.4 Hz, 1H), 7.44-7.40 (m, 2H), 7.13 (t, J=8.8 Hz, 2H), 5.13-5.04 (m, 1H), 4.77-4.62 (m, 2H), 3.08-2.98 (m, 2H), 1.47 (d, J=6.9 Hz, 3H). MS m/z: 341.25.

### Example 53: (R)-N-(1-(4-fluorophenyl)ethyl)-5-(5-(2,2,2-trifluoroethyl)pyridin-3-yl)pyrazin-2-amine

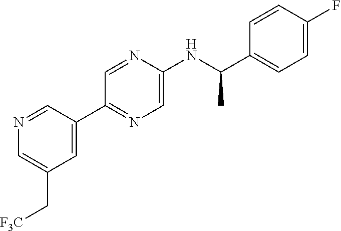 [SMILES: C[C@@H](Nc1cnc(-c2cncc(CC(F)(F)F)c2)cn1)c1ccc(F)cc1]

Tributyltin hydride (0.349 g, 1.2 mmol) was added to a stirred solution of O-phenyl O-(2,2,2-trifluoro-1-(5-(5-(((R)-1-(4-fluorophenyl)ethyl)amino)pyrazin-2-yl)pyridin-3-yl)ethyl) carbonothioate (Intermediate 23) (0.2 g, 0.4 mmol) and azobisisobutyronitrile (0.007 g, 0.06 mmol) in toluene (10 mL) at 0° C. The mixture was heated to 120° C. for 1 h and evaporated. Purification of the residue by column chromatography (amine silica, 50-55% EtOAc in petroleum ether) gave (R)-N-(1-(4-fluorophenyl)ethyl)-5-(5-(2,2,2-trifluoroethyl)pyridin-3-yl)pyrazin-2-amine (80 mg, 56%) as a white solid. ¹H NMR (400 MHz, DMSO-d₆): δ 9.06 (s, 1H), 8.60 (s, 1H), 8.48 (s, 1H), 8.26 (s, 1H), 8.10 (s, 1H), 7.90 (d, J=7.60 Hz, 1H), 7.44-7.40 (m, 2H), 7.13 (t, J=8.8 Hz, 2H), 5.10-5.06 (m, 1H), 3.78 (q, J=11.60 Hz, 2H), 1.46 (d, J=6.80 Hz, 3H). MS m/z: 377.24.

### Example 54: 2,2-difluoro-1-(5-(5-(((R)-1-(4-fluorophenyl)ethyl)amino)pyrazin-2-yl)pyridin-3-yl)ethan-1-ol

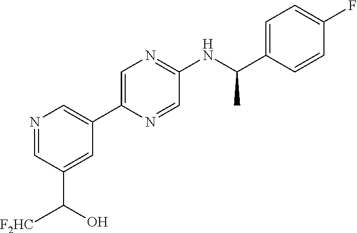

(Difluoromethyl)trimethylsilane (0.528 g, 4.2 mmol) was added to a stirred solution of (R)-5-(5-((1-(4-fluorophenyl)ethyl)amino)pyrazin-2-yl)nicotinaldehyde (Intermediate 26) (0.45 g, 1.4 mmol) in THF. The mixture was treated with TBAF (0.036 g, 0.14 mmol) at 0° C., stirred at RT for 12 h, treated with additional (difluoromethyl)trimethylsilane (0.352, 2.8 mmol) and TBAF (0.036 g, 0.14 mmol) and heated at 80° C. for 2 h. The mixture was quenched by slow addition of ice H₂O at 0° C. and extracted by EtOAc (2×50 mL). The combined organic layers were dried over anhydrous Na₂SO₄ and evaporated. Purification of the residue by column chromatography (ca. 20% EtOAc in petroleum ether) gave 2,2-difluoro-1-(5-(5-(((R)-1-(4-fluorophenyl)ethyl)amino)pyrazin-2-yl)pyridin-3-yl)ethan-1-ol (0.22 g, 47%) as a mixture of diastereomers, which were separated by SFC (CHIRALCEL-OX-H (30×250) mm, 5 μm; 78% CO₂, 22% MeOH; total flow: 110 g/min; back pressure: 100 bar) to the individual diastereomers.

Example 54a (Isomer 1): ¹H NMR (400 MHz, DMSO-d₆): δ 9.04 (d, J=1.7 Hz, 1H), 8.58 (s, 1H), 8.52 (s, 1H), 8.28 (s, 1H), 8.10 (s, 1H), 7.88 (d, J=7.5 Hz, 1H), 7.44-7.41 (m, 2H), 7.14 (t, J=8.8 Hz, 2H), 6.40 (s, 1H), 6.14 (t, J=22.9 Hz, 1H), 5.11-5.04 (m, 1H), 4.93-4.91 (m, 1H), 1.47 (d, J=6.9 Hz, 3H). MS m/z: 375.13. Analyt. chiral SFC: 99.7% (4.54 min).

Example 54b (Isomer 2): ¹H NMR (400 MHz, DMSO-d₆): δ 9.04 (d, J=1.7 Hz, 1H), 8.58 (s, 1H), 8.52 (s, 1H), 8.28 (s, 1H), 8.10 (s, 1H), 7.88 (d, J=7.5 Hz, 1H), 7.44-7.41 (m, 2H), 7.14 (t, J=8.8 Hz, 2H), 6.40 (s, 1H), 6.14 (t, J=22.9 Hz, 1H), 5.09-5.04 (m, 1H), 4.93-4.87 (m, 1H), 1.47 (d, J=6.9 Hz, 3H). MS m/z: 375.13. Analyt. chiral SFC: 99.0% (5.57 min).

### Example 55: (R)-N-(1-(4-fluorophenyl)ethyl)-6-(5-(2,2,2-trifluoroethyl)pyridin-3-yl)-1,2,4-triazin-3-amine

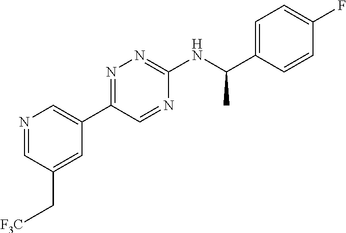

Tributyltin hydride (0.349 g, 1.2 mmol) was added to a stirred solution of O-phenyl O-(2,2,2-trifluoro-1-(5-(3-(((R)-1-(4-fluorophenyl)ethyl)amino)-1,2,4-triazin-6-yl)pyridin-3-yl)ethyl) carbonothioate (Intermediate 31) (0.15 g, 0.3 mmol) and azobisisobutyronitrile (0.0073 g, 0.06 mmol) in toluene (10 mL) at 0° C. and the resulting mixture was heated at 120° C. for 1 h. The solvent was evaporated and the residue purified by flash chromatography (Davisil silica, petroleum ether/EtOAc) to give (R)-N-(1-(4-fluorophenyl)ethyl)-6-(5-(2,2,2-trifluoroethyl)pyridin-3-yl)-1,2,4-triazin-3-amine as a pale yellow solid. (30 mg, 12%). ¹H NMR (400 MHz, DMSO-d₆): δ 9.12 (s, 1H), 8.90 (s, 1H), 8.62 (s, 1H), 8.40 (s, 1H), 7.46 (t, J=5.60 Hz, 2H), 7.14 (t, J=8.80 Hz, 2H), 5.20 (s, 1H), 3.83 (q, J=11.60 Hz, 2H), 1.51 (d, J=6.80 Hz, 3H). MS m/z: 378.21.

### Example 56: (R)-N-(1-(4-fluorophenyl)ethyl)-3-(5-(2,2,2-trifluoroethyl)pyridin-3-yl)-1,2,4-triazin-6-amine

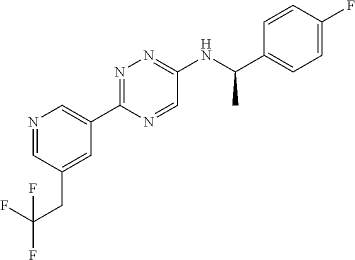 [SMILES: C[C@@H](Nc1cnc(-c2cncc(CC(F)(F)F)c2)nn1)c1ccc(F)cc1]

A mixture of 6-chloro-3-(5-(2,2,2-trifluoroethyl)pyridin-3-yl)-1,2,4-triazine (Intermediate 37) (27 mg, 0.98 mmol) and K₂CO₃ (41 mg, 0.295 mmol) in dioxane (3 mL) was treated with (R)-1-(4-fluorophenyl)ethan-1-amine (10 mg, 1.47 mmol), heated at 70° C. for 10 h, cooled to RT and filtered through Celite®. The filtrate was concentrated and the residue purified by prep. HPLC, followed by prep. TLC to give (R)-N-(1-(4-fluorophenyl)ethyl)-3-(5-(2,2,2-trifluoroethyl)pyridin-3-yl)-1,2,4-triazin-6-amine (1.5 mg, 2%). ¹H NMR (400 MHz, DMSO-d₆): δ 9.51 (s, 1H), 8.65-8.62 (m, 2H), 8.45 (s, 1H), 7.38-7.36 (m, 2H), 7.09-7.05 (m, 2H), 5.47 (br s, 1H), 5.34-5.31 (m, 1H), 3.49 (q, J=10.4 Hz, 1H), 1.42 (d, J=6.9 Hz, 3H). MS m/z: 378.35.

### Example 57: (R)-N-(1-(4-fluorophenyl)ethyl)-5-(6-(2,2,2-trifluoroethyl)pyridazin-4-yl)pyrazin-2-amine

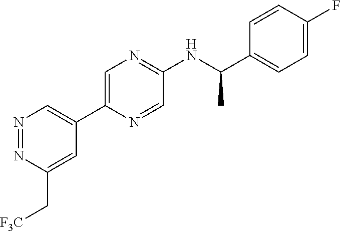 [SMILES: C[C@@H](Nc1cnc(-c2cnnc(CC(F)(F)F)c2)cn1)c1ccc(F)cc1]

A stirred solution of O-phenyl O-(2,2,2-trifluoro-1-(5-(5-(((R)-1-(4-fluorophenyl)ethyl)amino)pyrazin-2-yl)pyridazin-3-yl)ethyl) carbonothioate (Intermediate 35) (200 mg, 0.378 mmol) in toluene was treated with tributyltin hydride (440 mg, 1.51 mmol), azobisisobutyronitrile (6 mg, 0.038 mmol), stirred for 1 h at 100° C. and concentrated under reduced pressure. Purification of the residue by flash column chromatography (Davisil silica, 40-70% EtOAc in petroleum ether) gave (R)-N-(1-(4-fluorophenyl)ethyl)-5-(6-(2,2,2-trifluoroethyl)pyridazin-4-yl)pyrazin-2-amine as a pale brown solid (7 mg, 5%). ¹H NMR (400 MHz, DMSO-d₆): δ 9.75 (d, J=2.1 Hz, 1H), 8.82 (d, J=1.1 Hz, 1H), 8.27 (d, J=7.5 Hz, 1H), 8.16-8.15 (m, 2H), 7.44-7.40 (m, 2H), 7.14 (t, J=8.9 Hz, 2H), 5.15-5.11 (m, 1H), 4.08 (q, J=11.3 Hz, 2H), 1.48 (d, J=7.2 Hz, 3H). MS m/z: 378.35.

### Example 58: 2,2,2-trifluoro-1-(5-(5-(((R)-1-(4-fluorophenyl)ethyl)amino)pyrazin-2-yl)pyridazin-3-yl)ethan-1-ol

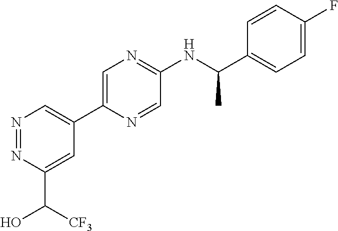 [SMILES: C[C@@H](Nc1cnc(-c2cnnc(C(O)C(F)(F)F)c2)cn1)c1ccc(F)cc1]

A solution of (R)-5-(5-((1-(4-fluorophenyl)ethyl)amino)pyrazin-2-yl)pyridazine-3-carbaldehyde (Intermediate 34) (400 mg, 1.24 mmol) in THF was treated with trimethyl(trifluoromethyl)silane (230 mg, 1.61 mmol) at 0° C., followed by TBAF (32 mg, 0.124 mmol), stirred for 16 h at RT and treated with H₂O. The mixture was extracted with EtOAc, the organic layer dried over Na₂SO₄ and concentrated under reduced pressure to give crude product. Purification by column chromatography (Davisil silica, 40-70% EtOAc in petroleum ether) gave 2,2,2-trifluoro-1-(5-(5-(((R)-1-(4-fluorophenyl)ethyl)amino)pyrazin-2-yl)pyridazin-3-yl)ethan-1-ol as a yellow solid (400 mg, 82%). ¹H NMR (400 MHz, DMSO-d₆): δ 9.79 (d, J=2.2 Hz, 1H), 8.86 (d, J=1.1 Hz, 1H), 8.29-8.24 (m, 1H), 8.16 (s, 1H), 7.44-7.37 (m, 1H), 7.14 (t, J=8.9 Hz, 1H), 5.45 (t, J=6.8 Hz, 1H), 5.13 (t, J=7.0 Hz, 1H), 1.49 (d, J=6.9 Hz, 3H). MS m/z: 394.15.

### Example 59: (R)-5-(6-((1-(4-fluorophenyl)ethyl)amino)pyridin-3-yl)benzo[d]oxazol-2(3H)-one

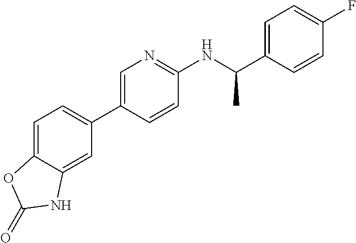 [SMILES: C[C@@H](Nc1ccc(-c2ccc3oc(=O)[nH]c3c2)cn1)c1ccc(F)cc1]

5-Bromobenzo[d]oxazol-2(3H)-one (0.1 g, 0.47 mmol), (R)-N-(1-(4-fluorophenyl)ethyl)-5-(4,4,5,5-tetramethyl-1,3,2-dioxaborolan-2-yl)pyridin-2-amine (Intermediate 3) (0.24 g, 0.70 mmol) and K₂CO₃ (0.130 g, 0.93 mmol) in dioxane (0.9 mL) and H₂O (0.1 mL) in a glass tube was purged with N₂ for 10 min. PdCl₂(dppf) (0.034 g, 0.047 mmol) was then added under N₂ atmosphere, the tube was sealed and the mixture was stirred at 100° C. for 16 h. The mixture was diluted with EtOAc (50 mL) and filtered through Celite®. The filtrate was washed with H₂O (20 mL). The organic layer was dried over anhydrous Na₂SO₄, filtered and concentrated under reduced pressure. Purification of the residue by flash column chromatography (0-80% EtOAc/petroleum ether) and re-purification by prep. HPLC (X bridge C18(19*250 mm); mobile phase A: 10 mM ammonium bicarbonate in H₂O, mobile phase B: CH₃CN, 0.01-17% B over 15 min; flow rate: 18 mL/min) gave (R)-5-(6-((1-(4-fluorophenyl)ethyl)amino)pyridin-3-yl)benzo[d]oxazol-2(3H)-one as colourless gum, which was lyophilized to give a white solid (19 mg, 11%). ¹H NMR (400 MHz, DMSO-d₆): δ 11.66 (s, 1H), 8.20 (d, J=2.0 Hz, 1H), 7.65-7.63 (m, 1H), 7.43-7.41 (m, 2H), 7.28 (d, J=8.3 Hz, 1H), 7.21-7.09 (m, J=8.2 Hz, 5H), 6.56 (d, J=8.7 Hz, 1H), 5.09-5.02 (m, 1H), 1.43 (d, J=6.9 Hz, 3H). MS m/z: 350.22 (M+H).

### Example 60: (R)-5-(6-((1-(4-fluorophenyl)ethyl)amino)pyridin-3-yl)-3-methylbenzo[d]oxazol-2(3H)-one

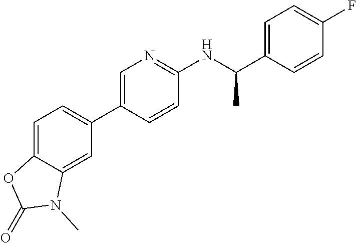 [SMILES: C[C@@H](Nc1ccc(-c2ccc3oc(=O)n(C)c3c2)cn1)c1ccc(F)cc1]

By following the procedure employed for Example 59 using 5-bromo-3-methylbenzo[d]oxazol-2(3H)-one (0.1 g, 0.44 mmol) and (R)-N-(1-(4-fluorophenyl)ethyl)-5-(4,4,5,5-tetramethyl-1,3,2-dioxaborolan-2-yl)pyridin-2-amine (Intermediate 3) (0.225 g, 0.66 mmol), (R)-5-(6-((1-(4-fluorophenyl)ethyl)amino)pyridin-3-yl)-3-methylbenzo[d]oxazol-2(3H)-one was obtained as off-white solid (44 mg, 27%). ¹H NMR (400 MHz, DMSO-d₆): δ 8.28 (d, J=2.0 Hz, 1H), 7.71-7.69 (m, 1H), 7.46 (s, 1H), 7.43-7.41 (m, 2H), 7.39-7.26 (m, 2H), 7.18 (d, J=7.7 Hz, 1H), 7.12 (t, J=8.8 Hz, 2H), 6.57 (d, J=8.7 Hz, 1H), 5.09-5.02 (m, 1H), 3.36 (s, 3H), 1.43 (d, J=6.9 Hz, 3H). MS m/z: 364.24.

### Example 61: (R)-6-(6-((1-(4-fluorophenyl)ethyl)amino)pyridin-3-yl)benzo[d]oxazol-2(3H)-one

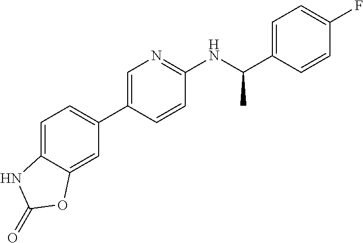 [SMILES: C[C@@H](Nc1ccc(-c2ccc3[nH]c(=O)oc3c2)cn1)c1ccc(F)cc1]

By following the procedure employed for Example 59 using 6-bromobenzo[d]oxazol-2(3H)-one (0.2 g, 0.90 mmol) and (R)-N-(1-(4-fluorophenyl)ethyl)-5-(4,4,5,5-tetramethyl-1,3,2-dioxaborolan-2-yl)pyridin-2-amine (Intermediate 3) (0.48 g, 1.40 mmol (R)-6-(6-((1-(4-fluorophenyl)ethyl)amino)pyridin-3-yl)benzo[d]oxazol-2(3H)-one was obtained as off-white solid (18 mg, 11%). ¹H NMR (400 MHz, DMSO-d₆): δ 11.62 (s, 1H), 8.21 (d, J=1.5 Hz, 1H), 7.65 (q, J=3.5 Hz, 1H), 7.41 (q, J=4.6 Hz, 3H), 7.26 (d, J=8.0 Hz, 1H), 7.08 (m, J=10.0 Hz, 4H), 6.54 (d, J=8.7 Hz, 1H), 5.05 (t, J=7.1 Hz, 1H), 1.42 (d, J=6.8 Hz, 3H). MS m/z: 350.23.

### Example 62: (R)-5-(5-((1-(4-fluorophenyl)ethyl)amino)pyrazin-2-yl)-3-methylbenzo[d]oxazol-2(3H)-one

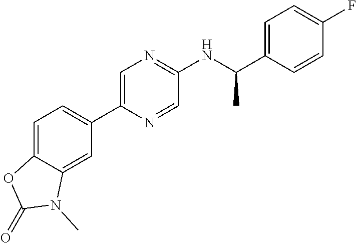 [SMILES: C[C@@H](Nc1cnc(-c2ccc3oc(=O)n(C)c3c2)cn1)c1ccc(F)cc1]

By following the procedure employed for Example 59 using 5-bromo-3-methylbenzo[d]oxazol-2(3H)-one (0.1 g, 0.44 mmol) and (R)-(5-((1-(4-fluorophenyl)ethyl)amino)pyrazin-2-yl)boronic acid (Intermediate 8 (0.18 g, 0.53 mmol), (R)-5-(5-((1-(4-fluorophenyl)ethyl)amino)pyrazin-2-yl)-3-methylbenzo[d]oxazol-2(3H)-one was obtained as white solid (34 mg, 21%). ¹H NMR (400 MHz, DMSO-d₆): δ 8.54 (d, J=1.2 Hz, 1H), 8.04 (d, J=1.2 Hz, 1H), 7.74 (q, J=4.1 Hz, 2H), 7.67 (q, J=3.4 Hz, 1H), 7.43 (q, J=4.7 Hz, 2H), 7.34 (d, J=8.4 Hz, 1H), 7.13 (t, J=8.9 Hz, 2H), 5.06 (t, J=7.1 Hz, 1H), 3.37 (s, 3H), 1.47 (d, J=6.9 Hz, 3H). MS m/z: 365.21.

### Example 63: (R)-5-(2-((1-(4-fluorophenyl)ethyl)amino)pyrimidin-5-yl)-3-methylbenzo[d]oxazol-2(3H)-one

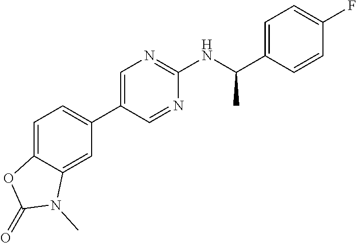 [SMILES: C[C@@H](Nc1ncc(-c2ccc3oc(=O)n(C)c3c2)cn1)c1ccc(F)cc1]

By following the procedure employed for Example 59 using 5-bromo-3-methylbenzo[d]oxazol-2(3H)-one (0.2 g, 0.88 mmol), (R)-N-(1-(4-fluorophenyl)ethyl)-5-(4,4,5,5-tetramethyl-1,3,2-dioxaborolan-2-yl)pyrimidin-2-amine (Intermediate 36) (0.45 g, 1.31 mmol) (R)-5-(2-((1-(4-fluorophenyl)ethyl)amino)pyrimidin-5-yl)-3-methylbenzo[d]oxazol-2(3H)-one was obtained as an off-white solid (82 mg, 26%). ¹H NMR (400 MHz, DMSO-d₆): δ 8.62 (br s, 2H), 7.92 (d, J=8.2 Hz, 1H), 7.55 (s, 1H), 7.44-7.41 (m, 2H), 7.37-7.32 (m, 2H), 7.12 (t, J=8.9 Hz, 2H), 5.14 (t, J=7.4 Hz, 1H), 3.39 (s, 3H), 1.45 (d, J=7.0 Hz, 3H). MS m/z: 365.25.

### Example 64: (R)-5-(3-((1-(4-fluorophenyl)ethyl)amino)-1,2,4-triazin-6-yl)-3-methylbenzo[d]oxazol-2(3H)-one

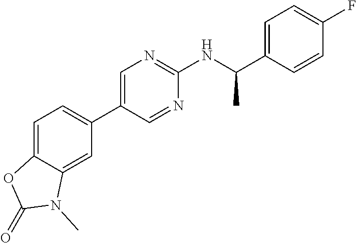 [SMILES: C[C@@H](Nc1ncc(-c2ccc3oc(=O)n(C)c3c2)cn1)c1ccc(F)cc1]

By following the procedure employed for Example 59 using (R)-6-chloro-N-(1-(4-fluorophenyl)ethyl)-1,2,4-triazin-3-amine (Intermediate 38) (2.2 g, 8.7 mmol) and 3-methyl-5-(4,4,5,5-tetramethyl-1,3,2-dioxaborolan-2-yl)benzo[d]oxazol-2(3H)-one (Intermediate 40) (3.35 g, 12.18 mmol), (R)-5-(3-((1-(4-fluorophenyl)ethyl)amino)-1,2,4-triazin-6-yl)-3-methylbenzo[d]oxazol-2(3H)-one was obtained as a pale brown solid (670 mg, 21%). ¹H NMR (400 MHz, DMSO-d₆): δ 8.87 (s, 1H), 8.48 (br s, 1H), 7.88 (d, J=1.60 Hz, 1H), 7.75 (dd, J=2.00, 8.40 Hz, 1H), 7.47-7.42 (m, 3H), 7.16-7.11 (m, 2H), 5.19 (s, 1H), 3.39 (s, 3H), 1.51 (d, J=7.20 Hz, 3H). MS m/z: 366.23.

### Example 65: (R)-5-(3-((1-(4-fluorophenyl)ethyl)amino)-1,2,4-triazin-6-yl)-3-(2-hydroxyethyl)benzo[d]oxazol-2(3H)-one

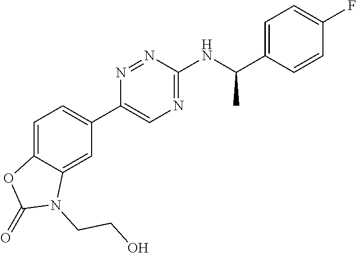 [SMILES: C[C@@H](Nc1ncc(-c2ccc3oc(=O)n(CCO)c3c2)nn1)c1ccc(F)cc1]

By following the procedure employed for Example 59 using (R)-6-chloro-N-(1-(4-fluorophenyl)ethyl)-1,2,4-triazin-3-amine (Intermediate 38) (90 mg, 0.4 mmol) and 3-(2-hydroxyethyl)-5-(4,4,5,5-tetramethyl-1,3,2-dioxaborolan-2-yl)benzo[d]oxazol-2(3H)-one (Intermediate 41) (170 mg, 0.56 mmol), (R)-5-(3-((1-(4-fluorophenyl)ethyl)amino)-1,2,4-triazin-6-yl)-3-(2-hydroxyethyl)benzo[d]oxazol-2(3H)-one was obtained as a pale brown solid (30 mg, 25%). ¹H NMR (400 MHz, DMSO-d₆): δ 8.85 (s, 1H), 8.48 (br s, 1H), 7.92 (s, 1H), 7.73 (d, J=8.3 Hz, 1H), 7.47-7.42 (m, 3H), 7.14 (t, J=8.8 Hz, 2H), 5.18 (s, 1H), 4.94 (t, J=5.7 Hz, 1H), 3.93-3.90 (m, 2H), 3.73-3.69 (m, 2H), 1.51 (d, J=6.9 Hz, 3H). MS m/z: 396.40.

### Example 66: (R)-5-(3-((1-(4-fluorophenyl)ethyl)amino)-1,2,4-triazin-6-yl)-3-(methoxymethyl)benzo[d]oxazol-2(3H)-one

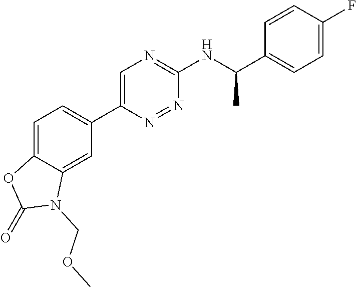 [SMILES: COCn1c(=O)oc2ccc(-c3cnc(N[C@H](C)c4ccc(F)cc4)nn3)cc21]

By following the procedure employed for Example 59 using (R)-6-chloro-N-(1-(4-fluorophenyl)ethyl)-1,2,4-triazin-3-amine (Intermediate 38) (300 mg, 1.19 mmol) and 3-(methoxymethyl)-5-(4,4,5,5-tetramethyl-1,3,2-dioxaborolan-2-yl)benzo[d]oxazol-2(3H)-one (Intermediate 47) (435 mg, 1.43 mmol), (R)-5-(3-((1-(4-fluorophenyl)ethyl)amino)-1,2,4-triazin-6-yl)-3-(methoxymethyl)benzo[d]oxazol-2(3H)-one was obtained as an off-white solid (90 mg, 19%). ¹H NMR (400 MHz, DMSO-d₆): δ 8.86 (s, 1H), 8.57 (br s, 1H), 7.97 (s, 1H), 7.79 (d, J=8.5 Hz, 1H), 7.49-7.44 (m, 3H), 7.14 (t, J=8.8 Hz, 2H), 5.28-5.18 (m, 3H), 1.51 (d, J=6.9 Hz, 3H). MS m/z: 396.23.

### Example 67: (R)-3-ethyl-5-(3-((1-(4-fluorophenyl)ethyl)amino)-1,2,4-triazin-6-yl)benzo[d]oxazol-2(3H)-one

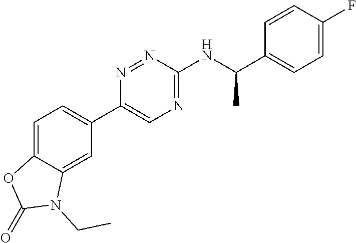 [SMILES: CCn1c(=O)oc2ccc(-c3cnc(N[C@H](C)c4ccc(F)cc4)nn3)cc21]

By following the procedure employed for Example 59 using (R)-6-chloro-N-(1-(4-fluorophenyl)ethyl)-1,2,4-triazin-3-amine (Intermediate 38) (100 mg, 0.4 mmol) and 3-ethyl-5-(4,4,5,5-tetramethyl-1,3,2-dioxaborolan-2-yl)benzo[d]oxazol-2(3H)-one (Intermediate 42) (161 mg, 1.4 mmol), (R)-3-ethyl-5-(3-((1-(4-fluorophenyl)ethyl)amino)-1,2,4-triazin-6-yl)benzo[d]oxazol-2(3H)-one was obtained as off-white solid (20 mg, 25%). ¹H NMR (400 MHz, DMSO-d₆): δ 8.88 (s, 1H), 8.48 (br s, 1H), 7.92 (s, 1H), 7.76 (d, J=8.3 Hz, 1H), 7.47-7.43 (m, 3H), 7.14 (t, J=8.7 Hz, 2H), 5.18 (s, 1H), 3.91 (q, J=7.0 Hz, 2H), 1.51 (d, J=6.9 Hz, 3H), 1.29 (t, J=7.1 Hz, 3H). MS m/z: 380.22.

### Example 68: (R)-3-(difluoromethyl)-5-(3-((1-(4-fluorophenyl)ethyl)amino)-1,2,4-triazin-6-yl)benzo[d]oxazol-2(3H)-one

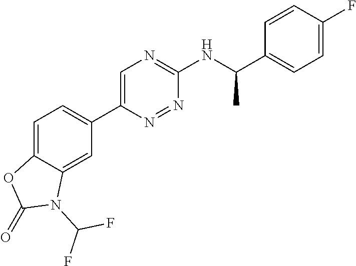 [SMILES: C[C@@H](Nc1ncc(-c2ccc3oc(=O)n(C(F)F)c3c2)nn1)c1ccc(F)cc1]

By following the procedure employed for Example 59 using (R)-6-chloro-N-(1-(4-fluorophenyl)ethyl)-1,2,4-triazin-3-amine (Intermediate 38) (70 mg, 0.187 mmol) and 3-(difluoromethyl)-5-(4,4,5,5-tetramethyl-1,3,2-dioxaborolan-2-yl)benzo[d]oxazol-2(3H)-one (Intermediate 43) (200 mg, 0.225 mmol), (R)-3-(difluoromethyl)-5-(3-((1-(4-fluorophenyl)ethyl)amino)-1,2,4-triazin-6-yl)benzo[d]oxazol-2(3H)-one was obtained an off-white solid (35 mg, 46%). ¹H NMR (400 MHz, DMSO-d₆): δ 8.89 (s, 1H), 8.54 (s, 1H), 7.99 (s, 1H), 7.92-7.63 (m, 2H), 7.58 (d, J=8.5 Hz, 1H), 7.47-7.42 (m, 2H), 7.14 (t, J=8.8 Hz, 2H), 5.19 (s, 1H), 1.51 (d, J=7.0 Hz, 3H). MS m/z: 402.2.

### Example 69: (R)-5-(3-((1-(4-fluorophenyl)ethyl)amino)-1,2,4-triazin-6-yl)benzo[d]oxazol-2(3H)-one

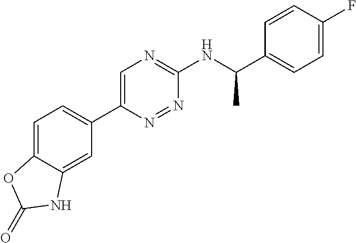 [SMILES: C[C@@H](Nc1ncc(-c2ccc3oc(=O)[nH]c3c2)nn1)c1ccc(F)cc1]

By following the procedure employed for Example 59 using (R)-6-chloro-N-(1-(4-fluorophenyl)ethyl)-1,2,4-triazin-3-amine (Intermediate 38) (207 mg, 0.7 mmol) and 5-(4,4,5,5-tetramethyl-1,3,2-dioxaborolan-2-yl)benzo[d]oxazol-2(3H)-one (255 mg, 0.98 mmol), (R)-5-(3-((1-(4-fluorophenyl)ethyl)amino)-1,2,4-triazin-6-yl)benzo[d]oxazol-2(3H)-one was obtained as off-white solid (21 mg, 14%). ¹H NMR (400 MHz, DMSO-d₆): δ 11.82 (s, 1H), 8.82 (s, 1H), 8.46 (br s, 1H), 7.69-7.66 (m, 2H), 7.47-7.37 (m, 3H), 7.14 (t, J=8.9 Hz, 2H), 5.19 (s, 1H), 1.50 (d, J=7.0 Hz, 3H). MS m/z: 352.38.

### Example 70: (R)-5-(3-((1-(4-fluorophenyl)ethyl)amino)-1,2,4-triazin-6-yl)-3-((methylthio)methyl)benzo[d]oxazol-2(3H)-one

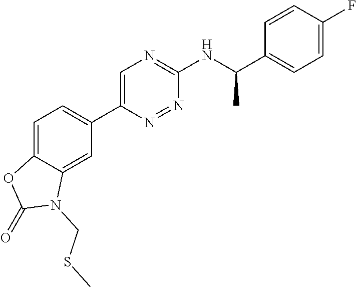 [SMILES: CSCn1c(=O)oc2ccc(-c3cnc(N[C@H](C)c4ccc(F)cc4)nn3)cc21]

By following the procedure employed for Example 59 using (R)-6-chloro-N-(1-(4-fluorophenyl)ethyl)-1,2,4-triazin-3-amine (Intermediate 38) (200 mg, 0.8 mmol) and 3-((methylthio)methyl)-5-(4,4,5,5-tetramethyl-1,3,2-dioxaborolan-2-yl)benzo[d]oxazol-2(3H)-one (Intermediate 44) (358 mg, 1.12 mmol), (R)-5-(3-((1-(4-fluorophenyl)ethyl)amino)-1,2,4-triazin-6-yl)-3-((methylthio)methyl)benzo[d]oxazol-2(3H)-one was obtained as a pale brown solid (36 mg, 21%). ¹H NMR (400 MHz, DMSO-d₆): δ 8.85 (s, 1H), 8.51 (br s, 1H), 8.04 (d, J=1.20 Hz, 1H), 7.78 (dd, J=1.60, 8.40 Hz, 1H), 7.48-7.44 (m, 3H), 7.14 (t, J=8.80 Hz, 2H), 5.19 (s, 1H), 5.09 (s, 2H), 2.16 (s, 3H), 1.51 (d, J=6.80 Hz, 3H). MS m/z: 412.2.

### Example 71: (R)-3-(2-(dimethylamino)ethyl)-5-(3-((1-(4-fluorophenyl)ethyl)amino)-1,2,4-triazin-6-yl)benzo[d]oxazol-2(3H)-one

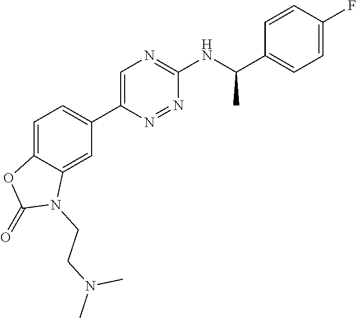 [SMILES: C[C@@H](Nc1ncc(-c2ccc3oc(=O)n(CCN(C)C)c3c2)nn1)c1ccc(F)cc1]

To a stirred solution of (R)-5-(3-((1-(4-fluorophenyl)ethyl)amino)-1,2,4-triazin-6-yl)benzo[d]oxazol-2(3H)-one (Example 69) (30 mg, 0.085 mmol) in CH₃CN (6 mL) in a glass tube was added 2-chloro-N,N-dimethylethan-1-amine (9.2 mg, 0.085 mmol) and K₂CO₃ (23.5 mg, 0.17 mmol). The mixture was heated at 110° C. while stirring for 12 h. The solvent was evaporated, the residue was suspended in H₂O and extracted with EtOAc. The extracts were combined, dried over anhydrous Na₂SO₄ and concentrated. The crude material was purified by prep-HPLC to furnish (R)-3-(2-(dimethylamino)ethyl)-5-(3-((1-(4-fluorophenyl)ethyl)amino)-1,2,4-triazin-6-yl)benzo[d]oxazol-2(3H)-one as a pale brown solid (8 mg, 22%). ¹H NMR (400 MHz, DMSO-d₆): δ 8.86 (s, 1H), 8.47 (br s, 1H), 7.91 (d, J=1.20 Hz, 1H), 7.75 (d, J=8.4 Hz, 1H), 7.47-7.42 (m, 3H), 7.13 (t, J=8.80 Hz, 2H), 5.47 (s, 1H), 5.18 (s, 1H), 3.98 (br s, 2H), 2.53-2.51 (m, 2H), 2.25-2.17 (m, 5H), 1.51 (d, J=6.80 Hz, 3H). MS m/z: 423.26

### Example 72: (R)-5-(3-((1-(4-fluorophenyl)ethyl)amino)-1,2,4-triazin-6-yl)-3-((methylsulfonyl)methyl)benzo[d]oxazol-2(3H)-one

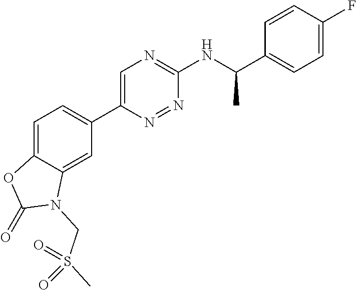 [SMILES: C[C@@H](Nc1ncc(-c2ccc3oc(=O)n(CS(C)(=O)=O)c3c2)nn1)c1ccc(F)cc1]

By following the procedure employed for Example 59 using (R)-6-chloro-N-(1-(4-fluorophenyl)ethyl)-1,2,4-triazin-3-amine (Intermediate 38) (150 mg, 0.6 mmol) and 3-((methylsulfonyl)methyl)-5-(4,4,5,5-tetramethyl-1,3,2-dioxaborolan-2-yl)benzo[d]oxazol-2(3H)-one (Intermediate 45), (R)-5-(3-((1-(4-fluorophenyl)ethyl)amino)-1,2,4-triazin-6-yl)-3-((methylsulfonyl)methyl)benzo[d]oxazol-2(3H)-one was obtained as a pale brown solid (8 mg, 3%). ¹H NMR (400 MHz, DMSO-d₆): δ 8.82 (s, 1H), 8.59 (br s, 1H), 7.79 (d, J=1.20 Hz, 1H), 7.53-7.44 (m, 3H), 7.13 (t, J=8.80 Hz, 2H), 5.47 (s, 2H), 5.18 (s, 1H), 3.14 (s, 3H) 1.51 (d, J=6.80 Hz, 3H). MS m/z: 444.23.

### Example 73: (R)-5-(3-((1-(4-chlorophenyl)ethyl)amino)-1,2,4-triazin-6-yl)-3-methylbenzo[d]oxazol-2(3H)-one

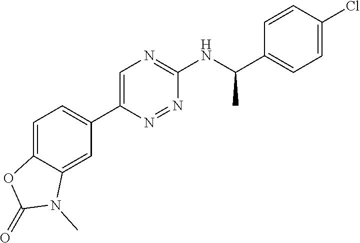 [SMILES: C[C@@H](Nc1ncc(-c2ccc3oc(=O)n(C)c3c2)nn1)c1ccc(Cl)cc1]

By following the procedure employed for Example 59 using (R)-6-chloro-N-(1-(4-chlorophenyl)ethyl)-1,2,4-triazin-3-amine (Intermediate 17) (160 mg, 0.594 mmol) and 3-methyl-5-(4,4,5,5-tetramethyl-1,3,2-dioxaborolan-2-yl)benzo[d]oxazol-2(3H)-one (Intermediate 40) (163 mg, 0.594 mmol), (R)-5-(3-((1-(4-chlorophenyl)ethyl)amino)-1,2,4-triazin-6-yl)-3-methylbenzo[d]oxazol-2(3H)-one was obtained as off-white solid (48 mg, 21%). ¹H NMR (400 MHz, DMSO-d₆): δ 8.87 (s, 1H), 8.50 (s, 1H), 7.88 (d, J=1.5 Hz, 1H), 7.76-7.73 (m, 1H), 7.45-7.41 (m, 3H), 7.38-7.36 (m, 2H), 5.16 (s, 1H), 3.39 (s, 3H), 1.51 (d, J=7.0 Hz, 3H). MS m/z: 382.88.

### Example 74: 4-(1-((6-(3-methyl-2-oxo-2,3-dihydrobenzo[d]oxazol-5-yl)-1,2,4-triazin-3-yl)amino)ethyl)benzonitrile

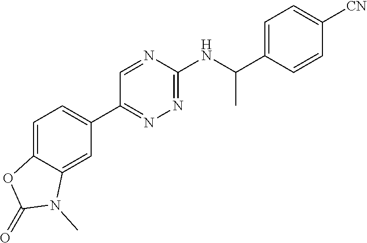 [SMILES: CC(Nc1ncc(-c2ccc3oc(=O)n(C)c3c2)nn1)c1ccc(C#N)cc1]

A stirred slurry of 5-(3-chloro-1,2,4-triazin-6-yl)-3-methylbenzo[d]oxazol-2(3H)-one (Intermediate 46) (100 mg, 0.381 mmol) in DMSO (4 mL) was treated with triethylamine (125 mg, 1.14 mmol) and 4-(1-aminoethyl)benzonitrile (61.25 mg, 0.419 mmol) and heated at 60° C. for 2 h. The mixture was cooled, treated with H₂O and extracted with EtOAc (2×20 mL). The combined organic layers were dried over Na₂SO₄, concentrated under reduced pressure and purified by prep. HPLC to obtain 4-(1-((6-(3-methyl-2-oxo-2,3-dihydrobenzo[d]oxazol-5-yl)-1,2,4-triazin-3-yl)amino)ethyl)benzonitrile as off-white solid (13 mg, 9.2%). ¹H NMR (400 MHz, DMSO-d₆): δ 8.88 (s, 1H), 8.58 (br s, 1H), 7.88 (d, J=1.4 Hz, 1H), 7.80-7.73 (m, 3H), 7.62 (d, J=8.2 Hz, 2H), 7.43 (d, J=8.4 Hz, 1H), 5.22 (s, 1H), 3.39 (s, 3H), 1.53 (d, J=7.0 Hz, 3H). MS m/z: 373.13.

### Example 75: (R)-5-(3-((1-(2,4-difluorophenyl)ethyl)amino)-1,2,4-triazin-6-yl)-3-methylbenzo[d]oxazol-2(3H)-one

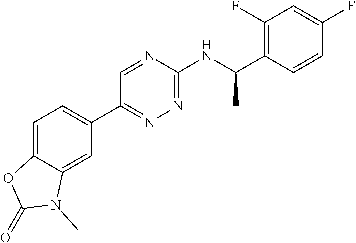 [SMILES: C[C@@H](Nc1ncc(-c2ccc3oc(=O)n(C)c3c2)nn1)c1ccc(F)cc1F]

By following the procedure described for Example 74 using 5-(3-chloro-1,2,4-triazin-6-yl)-3-methylbenzo[d]oxazol-2(3H)-one (Intermediate 46) (50 mg, 0.19 mmol) and (R)-1-(2,4-difluorophenyl)ethan-1-amine (42.43 mg, 0.27 mmol) followed by Prep-HPLC purification, gave (R)-5-(3-((1-(2,4-difluorophenyl)ethyl)amino)-1,2,4-triazin-6-yl)-3-methylbenzo[d]oxazol-2(3H)-one as off-white solid (3.5 mg, 5%). ¹H NMR (400 MHz, DMSO-d₆): δ 8.89 (s, 1H), 8.57 (s, 1H), 7.89 (d, J=1.4 Hz, 1H), 7.76-7.74 (m, 1H), 7.54-7.50 (m, 1H), 7.43 (d, J=8.4 Hz, 1H), 7.23-7.17 (m, 1H), 7.07-7.02 (m, 1H), 5.40 (s, 1H), 3.39 (s, 3H), 1.51 (d, J=7.0 Hz, 3H). MS m/z: 384.36.

### Example 76: (R)-5-(3-((1-(3,4-difluorophenyl)ethyl)amino)-1,2,4-triazin-6-yl)-3-methylbenzo[d]oxazol-2(3H)-one

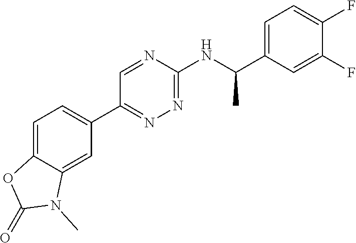 [SMILES: C[C@@H](Nc1ncc(-c2ccc3oc(=O)n(C)c3c2)nn1)c1ccc(F)c(F)c1]

By following the procedure described for Example 74 using 5-(3-chloro-1,2,4-triazin-6-yl)-3-methylbenzo[d]oxazol-2(3H)-one (Intermediate 46) (50 mg, 0.19 mmol) and (R)-1-(3,4-difluorophenyl)ethan-1-amine (42.43 mg, 0.27 mmol) followed by Prep-HPLC purification, gave (R)-5-(3-((1-(3,4-difluorophenyl)ethyl)amino)-1,2,4-triazin-6-yl)-3-methylbenzo[d]oxazol-2(3H)-one as an off-white solid (13 mg, 18%). ¹H NMR (400 MHz, DMSO-d₆): δ 8.89 (s, 1H), 8.48 (br s, 1H), 7.89 (d, J=1.5 Hz, 1H), 7.77-7.74 (m, 1H), 7.50-7.36 (m, 3H), 7.28 (br s, 1H), 5.18 (s, 1H), 3.39 (s, 3H), 1.51 (d, J=7.0 Hz, 3H). MS m/z: 384.36 (M+H).

### Example 77: 5-(3-((1-(4-fluoro-3-methylphenyl)ethyl)amino)-1,2,4-triazin-6-yl)-3-methylbenzo[d]oxazol-2(3H)-one

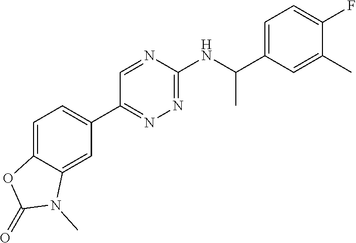 [SMILES: Cc1cc(C(C)Nc2ncc(-c3ccc4oc(=O)n(C)c4c3)nn2)ccc1F]

By following the procedure described for Example 74 using 5-(3-chloro-1,2,4-triazin-6-yl)-3-methylbenzo[d]oxazol-2(3H)-one (Intermediate 46) (100 mg, 0.762 mmol) and 1-(4-fluoro-3-methylphenyl)ethan-1-amine (174.6 mg, 1.14 mmol). Separation of the racemate by chiral preparative SFC (Chiralcel OX-H, 21×250 mm, 5 μm; 60% CO₂, 40% of 0.2% isopropylamine in isopropanol; total flow: 60 g/min; back pressure: 100 bar) gave 5-(3-((1-(4-fluoro-3-methylphenyl)ethyl)amino)-1,2,4-triazin-6-yl)-3-methylbenzo[d]oxazol-2(3H)-one as an off-white solid (39 mg, 27%).

Example 77a (Isomer 1) was obtained as an off-white solid (39 mg, 27%). ¹H NMR (400 MHz, DMSO-d₆): δ 8.87 (s, 1H), 8.45 (s, 1H), 7.88 (d, J=1.4 Hz, 1H), 7.76-7.44 (m, 1H), 7.43 (d, J=8.4 Hz, 1H), 7.33-25 (m, 1H), 7.06 (t, J=9.1 Hz, 1H), 5.14 (s, 1H), 3.39 (s, 3H), 2.21 (s, 3H), 1.49 (d, J=7.0 Hz, 3H). MS m/z: 380.24. Chiral SFC: Rt 5.61 min, chiral purity 99.8%.

Example 77b (Isomer 2) was obtained as an off-white solid (36 mg, 25%). ¹H NMR (400 MHz, DMSO) δ 8.86 (s, 1H), 8.43 (br s, 1H), 7.88 (d, J=1.6 Hz, 1H), 7.76-7.74 (dd, J=1.6 Hz, 8.4 Hz, 1H), 7.43 (d, J=7.6 Hz, 1H), 7.32 (d, J=7.6 Hz, 1H), 7.27-7.24 (m, 1H), 7.06 (t, J=8.8 Hz, 1H), 5.15 (br s, 1H), 3.39 (s, 3H), 2.21 (s, 3H), 1.49 (d, J=6.8 Hz, 3H). MS m/z: 380.28. HPLC: 99.7%. Chiral SFC: Rt 3.57 min, chiral purity: 99.8%.

### Example 78: 5-(3-((1-(3,4-dichlorophenyl)ethyl)amino)-1,2,4-triazin-6-yl)-3-methylbenzo[d]oxazol-2(3H)-one

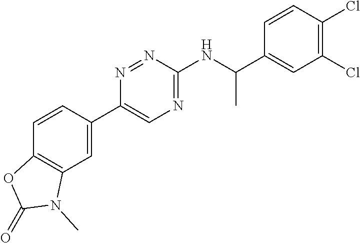 [SMILES: CC(Nc1ncc(-c2ccc3oc(=O)n(C)c3c2)nn1)c1ccc(Cl)c(Cl)c1]

By following the procedure described for Example 74 using 5-(3-chloro-1,2,4-triazin-6-yl)-3-methylbenzo[d]oxazol-2(3H)-one (Intermediate 46) (75 mg, 0.281 mmol) and 1-(3,4-dichlorophenyl)ethan-1-amine (41.86 mg, 0.34 mmol), 5-(3-((1-(3,4-dichlorophenyl)ethyl)amino)-1,2,4-triazin-6-yl)-3-methylbenzo[d]oxazol-2(3H)-one was obtained. The racemate was separated by chiral preparative SFC (Chiralcel OX-H (21×250) mm, 5 μm; 60% CO₂, 40% MeOH, total flow: 60 g/min; back pressure: 100 bar).

Example 78a (Isomer 1) was obtained as a yellow solid (45 mg, 39%). ¹H NMR (400 MHz, DMSO-d₆): δ 8.89 (s, 1H), 8.52 (s, 1H), 7.89 (d, J=1.4 Hz, 1H), 7.77-7.74 (m, 1H), 7.69 (d, J=1.8 Hz, 1H), 7.59 (d, J=8.3 Hz, 1H), 7.44-7.40 (m, 2H), 5.17 (s, 1H), 3.39 (s, 3H), 1.51 (d, J=7.0 Hz, 3H). MS m/z: 416.09. Chiral SFC: Rt 5.22 min, chiral purity 99.8%.

Example 78b (Isomer 2) was obtained (34 mg, 29%) as a yellow solid. ¹H NMR (400 MHz, DMSO-d₆): δ 8.89 (s, 1H), 8.54 (br s, 1H), 7.89 (d, J=1.6 Hz, 1H), 7.77-7.74 (dd, J=1.6 Hz, 8.4 Hz, 1H), 7.69 (d, J=2.0 Hz, 1H), 7.59 (d, J=8.4 Hz, 1H), 7.44-7.40 (m, 2H), 5.17 (br s, 1H), 3.39 (s, 3H), 1.51 (d, J=7.2 Hz, 3H). MS m/z: 416.09. HPLC: 98.5%; Chiral SFC: Rt 2.99 min, chiral purity 95-5%.

### Example 79: (R)-3,3-difluoro-6-(6-((1-(4-fluorophenyl)ethyl)amino)pyridin-3-yl)-1-methylindolin-2-one

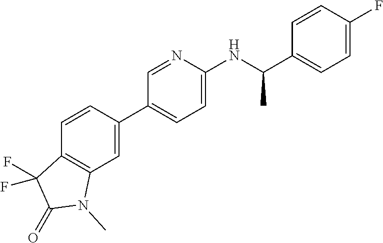 [SMILES: C[C@@H](Nc1ccc(-c2ccc3c(c2)N(C)C(=O)C3(F)F)cn1)c1ccc(F)cc1]

By following the procedure employed for Example 59 using 6-bromo-3,3-difluoro-1-methylindolin-2-one (Intermediate 48) (0.1 g, 0.3802 mmol) and (R)-N-(1-(4-fluorophenyl)ethyl)-5-(4,4,5,5-tetramethyl-1,3,2-dioxaborolan-2-yl)pyridin-2-amine (Intermediate 3) (0.160 g, 0.467 mmol), (R)-3,3-difluoro-6-(6-((1-(4-fluorophenyl)ethyl)amino)pyridin-3-yl)-1-methylindolin-2-one was obtained as a yellow solid (40 mg, 27%). ¹H NMR (400 MHz, DMSO) δ 8.41 (s, 1H), 7.81 (d, J=8.0 Hz, 1H), 7.66 (d, J=8.0 Hz, 1H), 7.43-7.38 (m, 5H), 7.12 (t, J=8.8 Hz, 2H), 6.60 (d, J=8.0 Hz, 1H), 5.08 (t, J=6.8 Hz, 1H), 3.22 (s, 3H), 1.44 (d, J=6.8 Hz, 3H). MS m/z: 398.52 (M+H).

### Example 80: (R)-6-(benzo[d]oxazol-5-yl)-N-(1-(4-fluorophenyl)ethyl)-1,2,4-triazin-3-amine

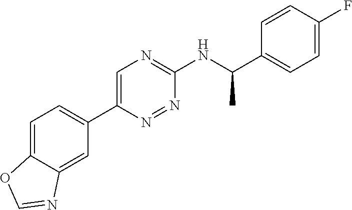 [SMILES: C[C@@H](Nc1ncc(-c2ccc3ocnc3c2)nn1)c1ccc(F)cc1]

By following the procedure employed for Example 59 using (R)-6-chloro-N-(1-(4-fluorophenyl)ethyl)-1,2,4-triazin-3-amine (Intermediate 38) (70 mg, 0.28 mmol) and 5-(4,4,5,5-tetramethyl-1,3,2-dioxaborolan-2-yl)benzo[d]oxazole (200 mg, 0.64 mmol), (R)-6-(benzo[d]oxazol-5-yl)-N-(1-(4-fluorophenyl)ethyl)-1,2,4-triazin-3-amine was obtained as an off-white solid (6 mg, 6%). ¹H NMR (400 MHz, DMSO) δ 8.93 (s, 1H), 8.81 (s, 1H), 8.47 (br s, 1H), 8.38 (s, 1H), 8.11-8.08 (dd J=1.6 Hz and 8.8 Hz, 1H), 7.88 (d, J=8.8 Hz, 1H), 7.49-7.45 (m, 2H), 7.14 (t, J=8.8 Hz, 2H), 5.21 (br s, 1H), 1.51 (d, J=6.8 Hz, 3H). MS m/z: 336.28 (M+H).

### Example 81: (R)-N-(1-(4-fluorophenyl)ethyl)-6-(1-methyl-1H-benzo[d]imidazol-6-yl)-1,2,4-triazin-3-amine

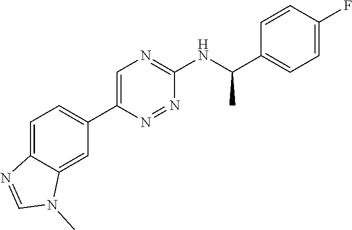 [SMILES: C[C@@H](Nc1ncc(-c2ccc3ncn(C)c3c2)nn1)c1ccc(F)cc1]

By following the procedure employed for Example 59 using (R)-6-chloro-N-(1-(4-fluorophenyl)ethyl)-1,2,4-triazin-3-amine (Intermediate 38) (90 mg, 0.349 mmol) and 1-methyl-6-(4,4,5,5-tetramethyl-1,3,2-dioxaborolan-2-yl)-1H-benzo[d]imidazole (88 mg, 0.349 mmol) (Intermediate 49), (R)-N-(1-(4-fluorophenyl)ethyl)-6-(1-methyl-1H-benzo[d]imidazol-6-yl)-1,2,4-triazin-3-amine was obtained as an off white solid (60 mg, 49%). ¹H NMR (400 MHz, DMSO): δ 8.92 (s, 1H), 8.41 (br s, 1H), 8.24 (s, 1H), 8.21 (d, J=1.2 Hz, 1H), 7.87-7.84 (dd, J=1.6 Hz and 8.8 Hz, 1H), 7.72 (d, J=8.4 Hz, 1H), 7.49-7.45 (m, 2H), 7.14 (t, J=8.8 Hz, 2H), 5.20 (br s, 1H), 3.89 (s, 3H), 1.51 (d, J=6.8 Hz, 3H). MS m/z: 349.26 (M+H).

### Example 82: (R)-N-(1-(4-fluorophenyl)ethyl)-6-(1-methyl-1H-imidazo[4,5-b]pyridin-6-yl)-1,2,4-triazin-3-amine

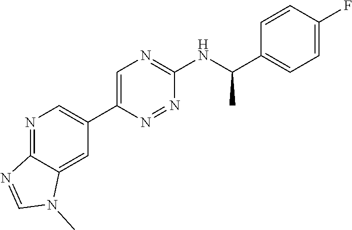 [SMILES: C[C@@H](Nc1ncc(-c2cnc3ncn(C)c3c2)nn1)c1ccc(F)cc1]

By following the procedure employed for Example 59 using (R)-6-chloro-N-(1-(4-fluorophenyl)ethyl)-1,2,4-triazin-3-amine (Intermediate 38) (60 mg, 0.237 mmol) and (1-methyl-1H-imidazo[4,5-b]pyridin-6-yl)boronic acid (Intermediate 50) (91 mg, 0.513 mmol), (R)-N-(1-(4-fluorophenyl)ethyl)-6-(1-methyl-1H-imidazo[4,5-b]pyridin-6-yl)-1,2,4-triazin-3-amine was obtained as an off-white solid (6.8 mg, 8%). ¹H NMR (400 MHz, DMSO) δ 9.02 (d, J=2.0 Hz, 1H), 8.96 (s, 1H), 8.62 (d, J=2.0 Hz, 1H), 8.49 (s, 2H), 7.49-7.45 (m, 2H), 7.14 (t, J=8.8 Hz, 2H), 5.20 (br s, 1H), 3.92 (s, 3H), 1.53 (d, J=5.4 Hz, 3H). MS m/z: 350.22 (M+H).

### Example 83: (R)-N-(1-(4-fluorophenyl)ethyl)-6-(3-methyl-3H-imidazo[4,5-b]pyridin-6-yl)-1,2,4-triazin-3-amine

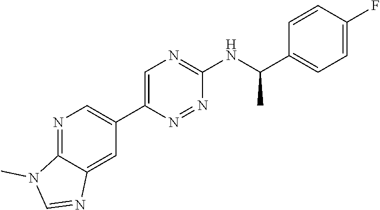 [SMILES: C[C@@H](Nc1ncc(-c2cnc3c(c2)ncn3C)nn1)c1ccc(F)cc1]

By following the procedure employed for Example 59 using (R)-6-chloro-N-(1-(4-fluorophenyl)ethyl)-1,2,4-triazin-3-amine (Intermediate 38) (60 mg, 0.237 mmol), (3-methyl-3H-imidazo[4,5-b]pyridin-6-yl)boronic acid (91 mg, 0.513 mmol) (Intermediate 51), (R)-N-(1-(4-fluorophenyl)ethyl)-6-(3-methyl-3H-imidazo[4,5-b]pyridin-6-yl)-1,2,4-triazin-3-amine was obtained as a brown solid (6.8 mg, 8%). ¹H NMR (400 MHz, DMSO) δ 8.99 (d, J=1.9 Hz, 1H), 8.94 (s, 1H), 8.59 (d, J=2.0 Hz, 1H), 8.49 (s, 2H), 7.49-7.45 (m, 2H), 7.14 (t, J=9.2 Hz, 2H), 5.20 (br s, 1H), 3.87 (s, 3H), 1.52 (d, J=7.2 Hz, 3H). MS m/z: 350.22 (M+H).

### 3. Biological Assays

### Ca²⁺ Mobilization Assay

This cell-based assay measures the ability of compounds to activate the GPR55 receptor by measuring changes in intracellular calcium levels. HEK293 (human embryonic kidney) cells co-expressing recombinant ProLink (PK) tagged human or rat GPR55 and Enzyme Acceptor (EA) tagged beta-arrestin were seeded into PDL-coated, black-walled clear-bottom 384 well plates in assay buffer (25 mM HEPES, HBSS, 1 mM Probenicid, 0.1% BSA, pH 7.4) containing Calcium5 dye (Molecular Devices). Cells were incubated in suspension with the Calcium5 dye for 1 hour at 37° C., 5% CO₂. Dilution series of the compounds in assay buffer were added to the cells in duplicate wells and compound-mediated real-time increases in intracellular calcium concentration were recorded as increased fluorescence using the Molecular Devices FLIPR instrument. Kinetic traces were reduced to a numerical value by subtracting the minimum response from the maximum response. EC₅₀ values were derived from this data using four-parameter logistic regression. The EC₅₀ values for the compounds of the examples are shown in Table 1.

TABLE 1

| | Human GPR55 | Rat GPR55 |
|---|---|---|
| Example | EC₅₀ (μM) | EC₅₀ (μM) |
| 1 | +++ | + |
| 2 | ++++ | + |
| 3 | ++++ | ++++ |
| 4 | ++++ | ++ |
| 5 | ++++ | ++ |
| 6 | +++ | ++++ |
| 7 | +++ | +++ |
| 8 | +++ | ++++ |
| 9 | +++ | ++++ |
| 10 | ++ | ++ |
| 11 | ++ | ++ |
| 12 | ++ | + |
| 13 | +++ | + |
| 14 | +++ | + |
| 15 | +++ | ++ |
| 16a | ++ | ++++ |
| 16b | ++++ | +++ |
| 17 | ++++ | ++ |
| 18 | ++++ | ++++ |
| 19 | ++ | ++ |
| 20 | ++++ | ++++ |
| 21 | +++ | + |
| 22 | +++ | + |
| 23 | ++ | + |
| 24 | +++ | + |
| 25 | ++++ | + |
| 26 | ++ | + |
| 27 | ++ | ++++ |
| 27a | ++ | +++ |
| 27b | +++ | ++ |
| 28 | ++++ | ++ |
| 29a | ++++ | ++ |
| 29b | ++ | + |
| 30 | ++++ | ++++ |
| 30a | + | ++++ |
| 30b | ++++ | ++ |
| 31 | ++++ | ++++ |
| 32 | +++ | ++++ |
| 32a | ++++ | ++++ |
| 32b | ++++ | ++ |
| 33 | ++++ | ++ |
| 34 | ++++ | ++ |
| 35 | ++++ | + |
| 36 | +++ | + |
| 37 | +++ | ++++ |
| 38 | ++ | ++++ |
| 39 | ++ | ++ |
| 40 | +++ | + |
| 41 | +++ | ++ |
| 42 | +++ | ++ |
| 43 | ++++ | + |
| 44 | ++ | + |
| 45 | +++ | ++ |
| 46a | ++ | +++ |
| 47 | ++ | + |
| 48 | ++++ | ++ |
| 49 | ++++ | ++ |
| 50 | ++ | + |
| 51 | ++++ | ++ |
| 52 | ++++ | +++ |
| 53 | +++ | + |
| 54a | ++ | +++ |
| 54b | ++ | ++ |
| 55 | ++ | + |
| 56 | ++ | + |
| 57 | ++++ | + |
| 58 | ++++ | + |
| 59 | ++ | + |
| 60 | ++ | ++ |
| 61 | ++ | + |
| 62 | ++ | ++ |
| 63 | ++ | ++ |
| 64 | +++ | +++ |
| 65 | ++ | + |
| 66 | +++ | ++ |
| 67 | +++ | ++ |
| 68 | +++ | ++ |
| 69 | ++ | ++ |
| 70 | +++ | +++ |
| 71 | ++ | + |
| 72 | ++ | + |
| 73 | ++ | + |
| 74 | ++ | + |
| 75 | ++ | ++ |
| 76 | ++ | ++ |
| 77a | ++ | ++ |
| 78a | ++ | ++ |
| 79 | + | + |
| 80 | +++ | ++ |
| 81 | ++ | + |
| 82 | ++ | + |
| 83 | + | + |

EC₅₀ (≤50 nM = ‘++++’; ≤200 nM = ‘+++’; ≤1000 nM = ‘++’; ≤5000 nM = ‘+’)

### Electrophysiology Assay

Selected compounds were tested in electrophysiology. These experiments show that modulating GPR55 alters firing rate of striatal medium spiny neurons.

Striatal cultures were prepared as follows. Wistar rat embryos (E18) were sacrificed by cervical dislocation and brains removed into an ice-cold hanks balanced salt solution (HBSS) containing 10 mM HEPES, 1 mM sodium pyruvate and 0.035% sodium bicarbonate. The brain was hemisected, striata removed and placed in a papain solution (Worthington LK003150) for 10 min at 37° C. Tissue was then vigorously triturated until devoid of clumps, passed through a 70 μm mesh filter and centrifuged at 1000 rcf for 5 min. Supernatant was discarded and cells resuspended and centrifuged once more through an ovomucoid gradient before final resuspension in plating medium (containing EMEM 75%, FBS 10%, glutamate 12.5 μM, GlutaMax 0.5 mM, sodium pyruvate 10 mM, glucose 33.3 mM, HEPES 10 nM). Cells were counted and spot plated onto 13 mm coverslips at a density of 200K. After 3 h, solution was topped up to 1 ml with growth medium (containing NBplus 98%, B27plus 2%, GlutaMax 0.5 mM). A half media change was done once per week, cells were left to mature and taken for recording from 10-20 days in vitro.

Electrophysiology experiments were performed at room temperature. Borosilicate glass recording electrodes had a resistance of 5-8 ME2 and were filled with an internal solution containing (in mM): 140 K-gluconate, 0.1 EGTA, 5 KCl, 5 Mg-ATP, 10 HEPES; pH 7.3 with KOH.

All pharmacological tools were diluted in a HEPES-buffered external solution (containing NaCl 145 mM, KCl 3 mM, HEPES 10 mM, glucose 10 mM, CaCl₂ 2 mM, MgCl₂ 1 mM, pH 7.3) applied to the cells via a perfusion pump at ˜2 ml/min. A period of 3 min was allowed prior to recording to allow adequate time for the substances to reach the bath.

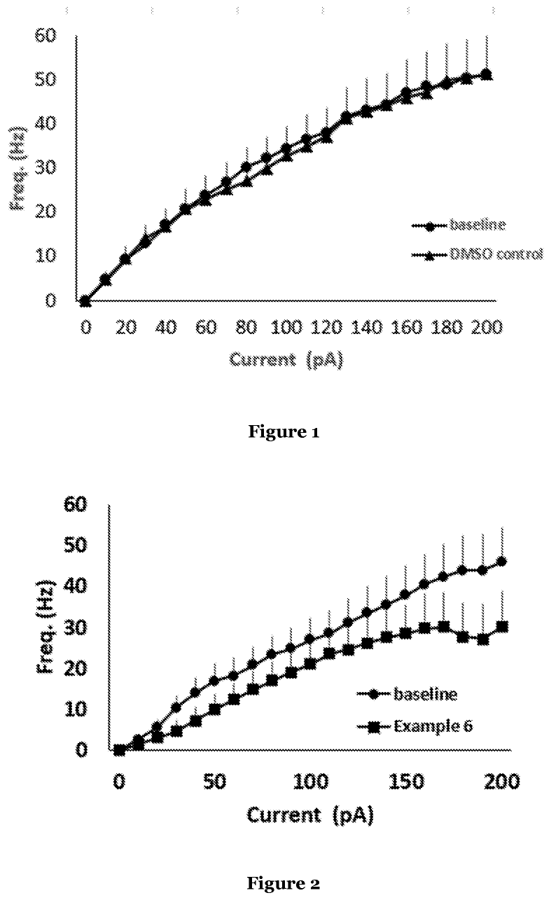

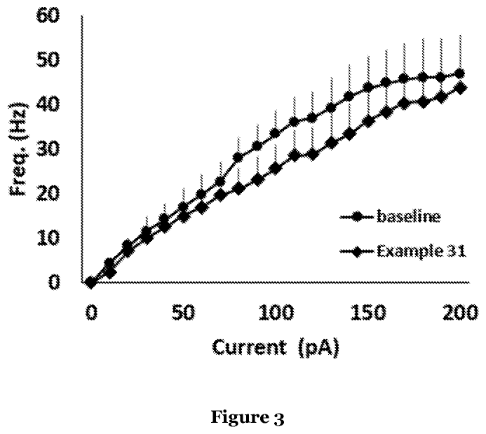

Whole-cell patch clamp recordings were performed from visually identified medium spiny neurons. A step protocol was applied to inject current into the cell (10-200 pA, 10 pA steps, 500 ms) and resulting action potentials recorded. After baseline recording, compound or vehicle control was applied to the bath and the step protocol repeated. Frequency of firing was analysed off-line using Clampfit software. FIG. 1 illustrates how DMSO had no effect on firing frequency of the medium spiny neurons while FIG. 2 and FIG. 3 illustrate that Example 6 and Example 31 significantly reduced the frequency.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 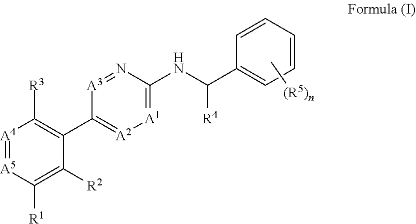
or a pharmaceutically acceptable salt, solvate or prodrug thereof, wherein:
A¹ is CH, CF or N;
A² is CH, CF or N;
A³ is CH, CMe, CCF₃ or N;
A⁴ is CH or N;
A⁵ is CH or N;
R¹ is halo, C₁-C₅ alkyl, C₂-C₅ alkenyl, C₂-C₅ alkynyl, C₃-C₅ cycloalkyl, —O(C₁-C₅ alkyl), —NH(C₁-C₃ alkyl), —N(C₁-C₃ alkyl)₂, —SO(C₁-C₃ alkyl), —SO₂(C₁-C₃ alkyl), —S(O)(NH)(C₁-C₃ alkyl) or —S(O)(N(C₁-C₃ alkyl))(C₁-C₃ alkyl), wherein any alkyl, alkenyl, alkynyl and cycloalkyl group is optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂;
or A⁵, R¹ and the carbon atom to which they are attached together form a 5-membered heterocyclic group containing 1 or 2 heteroatoms independently selected from N, O or S, wherein the heterocyclic group may contain an additional double bond, and wherein the heterocyclic group is optionally substituted with one or more substituents independently selected from halo, oxo (═O) or C₁-C₃ alkyl, wherein the C₁-C₃ alkyl is optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂;
R² is hydrogen or methyl;
R³ is hydrogen or methyl;
R⁴ is methyl;
R⁵ is independently halo, cyano, methyl or methoxy;
or, when a group R⁵ is in the ortho-position, R⁴ and R⁵ may together form a —CH₂CH₂— or —CH₂CH₂CH₂— group; and
n is 0, 1 or 2;
provided the compound is not:
5-(2-((1-phenylethyl)amino)pyrimidin-5-yl)-1,3-dihydro-2H-benzo[d]imidazol-2-one; or
5-(benzo[d][1,3]dioxol-5-yl)-N-(1-(4-fluorophenyl)ethyl)pyridin-2-amine.

2. The compound, salt, solvate or prodrug as claimed in claim 1, wherein the compound is of formula (Ia): 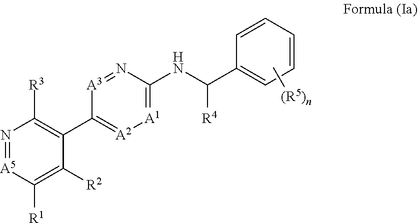
or a pharmaceutically acceptable salt, solvate or prodrug thereof, wherein:
A¹ is CH, CF or N;
A² is CH, CF or N;
A³ is CH, CMe, CCF₃ or N;
A⁵ is CH or N;
R¹ is halo, C₁-C₅ alkyl, C₂-C₅ alkenyl, C₂-C₅ alkynyl, C₃-C₅ cycloalkyl, —O(C₁-C₅ alkyl), —NH(C₁-C₃ alkyl), —N(C₁-C₃ alkyl)₂, —SO(C₁-C₃ alkyl), —SO₂(C₁-C₃ alkyl), —S(O)(NH)(C₁-C₃ alkyl) or —S(O)(N(C₁-C₃ alkyl))(C₁-C₃ alkyl), wherein any alkyl, alkenyl, alkynyl and cycloalkyl group is optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂;
R² is hydrogen or methyl;
R³ is hydrogen or methyl;
R⁴ is methyl;
R⁵ is independently halo, cyano, methyl or methoxy;
or, when a group R⁵ is in the ortho-position, R⁴ and R⁵ may together form a —CH₂CH₂— or —CH₂CH₂CH₂— group; and
n is 0, 1 or 2.

3. The compound, salt, solvate or prodrug as claimed in claim 1, wherein R¹ is fluoro, chloro, methyl, ethyl, vinyl, ethynyl, cyclopropyl, methoxy, ethoxy, —NHMe, —NMe₂, —SOMe, —SOEt, —SO₂Me, —SO₂Et, —S(O)(NH)Me or —S(O)(NMe)Me, each of which is optionally substituted with one, two, three or four substituents independently selected from fluoro, chloro, hydroxyl, —OMe, —SMe, —SOMe, —SO₂Me, —NHMe or —NMe₂.

4. The compound, salt, solvate or prodrug as claimed in claim 1, wherein the compound is of formula (Ib): 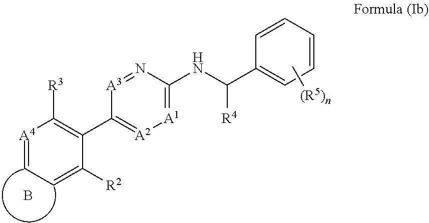
or a pharmaceutically acceptable salt, solvate or prodrug thereof, wherein:
A¹ is CH, CF or N;
A² is CH, CF or N;
A³ is CH, CMe, CCF₃ or N;
A⁴ is CH or N;
B is a 5-membered heterocyclic group containing 1 or 2 heteroatoms independently selected from N, O or S, wherein the heterocyclic group may contain an additional double bond, and wherein the heterocyclic group is optionally substituted with one or more substituents independently selected from halo, oxo (═O) or C₁-C₃ alkyl, wherein the C₁-C₃ alkyl is optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂;
R² is hydrogen or methyl;
R³ is hydrogen or methyl;
R⁴ is methyl;
R⁵ is independently halo, cyano, methyl or methoxy;
or, when a group R⁵ is in the ortho-position, R⁴ and R⁵ may together form a —CH₂CH₂— or —CH₂CH₂CH₂— group; and
n is 0, 1 or 2;
provided the compound is not:
5-(2-((1-phenylethyl)amino)pyrimidin-5-yl)-1,3-dihydro-2H-benzo[d]imidazol-2-one; or
5-(benzo[d][1,3]dioxol-5-yl)-N-(1-(4-fluorophenyl)ethyl)pyridin-2-amine.

5. The compound, salt, solvate or prodrug as claimed in claim 4, wherein B is a 5-membered heteroaryl group containing 1 or 2 heteroatoms independently selected from N, O or S, wherein the heteroaryl group is optionally substituted with one or two substituents independently selected from halo or C₁-C₃ alkyl, wherein the C₁-C₃ alkyl is optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂.

6. The compound, salt, solvate or prodrug as claimed in claim 5, wherein B is a pyrazolyl, imidazolyl, oxazolyl or isoxazolyl group, each of which is optionally substituted with fluoro, chloro or C₁-C₃ alkyl.

7. The compound, salt, solvate or prodrug as claimed in claim 4, wherein B is a ring represented by the group: 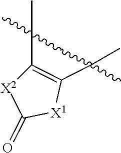
wherein:
X¹ is O, NH or NR⁷;
X² is O, NH, CF₂, CHF or CH₂; and
R⁷ is C₁-C₃ alkyl optionally substituted with one or more substituents independently selected from halo, hydroxyl, —O(C₁-C₃ alkyl), —S(C₁-C₃ alkyl), —SO(C₁-C₃ alkyl), —SO₂(C₁-C₃ alkyl), —NH(C₁-C₃ alkyl) or —N(C₁-C₃ alkyl)₂.

8. The compound, salt, solvate or prodrug as claimed in claim 7, wherein X¹ is NR⁷, and R⁷ is methyl or ethyl optionally substituted with one, two or three substituents independently selected from fluoro, chloro, hydroxyl, —OMe, —SMe, —SOMe, —SO₂Me, —NHMe or —NMe₂.

9. The compound, salt, solvate or prodrug as claimed in claim 7, wherein X² is O, NH or CF₂.

10. The compound, salt, solvate or prodrug as claimed in claim 1, wherein A¹ is CH or N, and/or wherein A³ is CH, CMe or N.

11. The compound, salt, solvate or prodrug as claimed in claim 1, wherein R⁵ is independently fluoro, chloro, cyano, methyl or methoxy, and/or wherein when a group R⁵ is in the ortho-position, R⁴ and R⁵ may together form a —CH₂CH₂— group.

12. The compound, salt, solvate or prodrug as claimed in claim 1, wherein the compound is selected from:
N-(1-(4-fluorophenyl)ethyl)-5′-methyl-[3,3′-bipyridin]-6-amine;
(R)-N-(1-(3,4-difluorophenyl)ethyl)-5′-methyl-[3,3′-bipyridin]-6-amine;
(R)-N-(1-(4-fluorophenyl)ethyl)-5′-methyl-[3,3′-bipyridin]-6-amine;
(R)-5′-ethyl-N-(1-(4-fluorophenyl)ethyl)-[3,3′-bipyridin]-6-amine;
(R)-5′-cyclopropyl-N-(1-(4-fluorophenyl)ethyl)-[3,3′-bipyridin]-6-amine;
(R)-N-(1-(4-fluorophenyl)ethyl)-5′-methoxy-[3,3′-bipyridin]-6-amine;
(R)-N-(1-(4-fluorophenyl)ethyl)-5′-(methylsulfonyl)-[3,3′-bipyridin]-6-amine;
(R)-N-(1-(4-fluorophenyl)ethyl)-5′-(trifluoromethyl)-[3,3′-bipyridin]-6-amine;
(R)-5′-chloro-N-(1-(4-fluorophenyl)ethyl)-[3,3′-bipyridin]-6-amine;
(R)-N-(1-(4-fluorophenyl)ethyl)-2′,5′-dimethyl-[3,3′-bipyridin]-6-amine;
(R)-N-(1-(4-fluorophenyl)ethyl)-4′,5′-dimethyl-[3,3′-bipyridin]-6-amine;
(R)-N6′-(1-(4-fluorophenyl)ethyl)-N5,N5-dimethyl-[3,3′-bipyridine]-5,6′-diamine;
5′-(ethylsulfinyl)-N-((R)-1-(4-fluorophenyl)ethyl)-[3,3′-bipyridin]-6-amine;
N-((R)-1-(4-fluorophenyl)ethyl)-5′-(methylsulfinyl)-[3,3′-bipyridin]-6-amine;
(R)-5′-(ethylsulfonyl)-N-(1-(4-fluorophenyl)ethyl)-[3,3′-bipyridin]-6-amine;
1-(6′-(((R)-1-(4-fluorophenyl)ethyl)amino)-[3,3′-bipyridin]-5-yl)ethan-1-ol;
(R)-5-(5-ethylpyridin-3-yl)-N-(1-(4-fluorophenyl)ethyl)pyrazin-2-amine;
(R)-N-(1-(4-fluorophenyl)ethyl)-5-(5-(methylsulfonyl)pyridin-3-yl)pyrazin-2-amine;
(R)-5′-ethyl-N-(1-(2-fluorophenyl)ethyl)-[3,3′-bipyridin]-6-amine;
(R)-N-(1-(4-fluorophenyl)ethyl)-5′-vinyl-[3,3′-bipyridin]-6-amine;
(R)-N-(2,3-dihydro-1H-inden-1-yl)-5′-ethyl-[3,3′-bipyridin]-6-amine;
(R)-N-(1-(4-fluorophenyl)ethyl)-2-methyl-5′-(methylsulfonyl)-[3,3′-bipyridin]-6-amine;
N-((R)-1-(4-fluorophenyl)ethyl)-5-(5-(methylsulfinyl)pyridin-3-yl)pyrazin-2-amine;
5-(5-(ethylsulfinyl)pyridin-3-yl)-N-((R)-1-(4-fluorophenyl)ethyl)pyrazin-2-amine;
(R)-N-(1-(4-fluorophenyl)ethyl)-5-(5-(methylsulfonyl)pyridin-3-yl)pyrimidin-2-amine;
(R)-6-(5-((1-(4-fluorophenyl)ethyl)amino)pyrazin-2-yl)-1-methyloxazolo[5,4-b]pyridin-2(1H)-one;
(6′-(((R)-1-(4-fluorophenyl)ethyl)amino)-[3,3′-bipyridin]-5-yl)(imino)(methyl)-λ⁶-sulfanone;
(R)-N-(1-(4-fluorophenyl)ethyl)-6-(5-(methylsulfonyl)pyridin-3-yl)-1,2,4-triazin-3-amine;
4-fluoro-N-(1-(4-fluorophenyl)ethyl)-5′-(methylsulfonyl)-[3,3′-bipyridin]-6-amine;
(6′-(((R)-1-(4-fluorophenyl)ethyl)amino)-[3,3′-bipyridin]-5-yl)(methyl)(methylimino)-λ6-sulfanone;
(R)-5-(5-(2,2-difluoroethyl)pyridin-3-yl)-N-(1-(4-fluorophenyl)ethyl)pyrazin-2-amine;
2,2,2-trifluoro-1-(5-(5-(((R)-1-(4-fluorophenyl)ethyl)amino)pyrazin-2-yl)pyridin-3-yl)ethan-1-ol;
(R)-N-(1-(4-fluorophenyl)ethyl)-5-(5-vinylpyridin-3-yl)pyrazin-2-amine;
(R)-5-(5-(2,2-difluorovinyl)pyridin-3-yl)-N-(1-(4-fluorophenyl)ethyl)pyrazin-2-amine;
(R)-2-(5-(5-((1-(4-fluorophenyl)ethyl)amino)pyrazin-2-yl)pyridin-3-yl)ethan-1-ol;
(R)-N-(2,3-dihydro-1H-inden-1-yl)-5-(5-(methylsulfonyl)pyridin-3-yl)pyrazin-2-amine;
(R)-N-(1-(4-fluorophenyl)ethyl)-5-(5-(methylamino)methylpyridin-3-yl)pyrazin-2-amine;
N-(5-fluoro-2,3-dihydro-1H-inden-1-yl)-5′-(methylsulfonyl)-[3,3′-bipyridin]-6-amine;
(R)-5-(5-(difluoromethoxy)pyridin-3-yl)-N-(1-(4-fluorophenyl)ethyl)pyrazin-2-amine;
(R)-6-(5-(2,2-difluorovinyl)pyridin-3-yl)-N-(1-(4-fluorophenyl)ethyl)-1,2,4-triazin-3-amine;
(R)-(5-(3-((1-(4-fluorophenyl)ethyl)amino)-1,2,4-triazin-6-yl)pyridin-3-yl)methanol;
(R)-N-(1-(4-fluorophenyl)ethyl)-5-(5-(2,2,2-trifluoroethoxy)pyridin-3-yl)pyrazin-2-amine;
(R)-N-(1-(4-fluorophenyl)ethyl)-5-(5-(trifluoromethoxy)pyridin-3-yl)pyrazin-2-amine;
(R)-N-(1-(4-fluorophenyl)ethyl)-5-(6-vinylpyridazin-4-yl)pyrazin-2-amine;
1-(5-(5-(((R)-2,3-dihydro-1H-inden-1-yl)amino)pyrazin-2-yl)pyridin-3-yl)-2,2-difluoroethan-1-ol;
N-(1-(4-chlorophenyl)ethyl)-5′-methyl-[3,3′-bipyridin]-6-amine;
(R)-5′-ethyl-N-(1-(4-methoxyphenyl)ethyl)-[3,3′-bipyridin]-6-amine;
(R)-5′-ethyl-N-(1-(3-fluorophenyl)ethyl)-[3,3′-bipyridin]-6-amine;
(R)-5′-ethyl-N-(1-phenylethyl)-[3,3′-bipyridin]-6-amine;
(R)-5′-ethyl-N-(1-(p-tolyl)ethyl)-[3,3′-bipyridin]-6-amine;
(R)-5′-ethynyl-N-(1-(4-fluorophenyl)ethyl)-[3,3′-bipyridin]-6-amine;
(R)-5-(5-(2-fluoroethyl)pyridin-3-yl)-N-(1-(4-fluorophenyl)ethyl)pyrazin-2-amine;
(R)-N-(1-(4-fluorophenyl)ethyl)-5-(5-(2,2,2-trifluoroethyl)pyridin-3-yl)pyrazin-2-amine;
2,2-difluoro-1-(5-(5-(((R)-1-(4-fluorophenyl)ethyl)amino)pyrazin-2-yl)pyridin-3-yl)ethan-1-ol;
(R)-N-(1-(4-fluorophenyl)ethyl)-6-(5-(2,2,2-trifluoroethyl)pyridin-3-yl)-1,2,4-triazin-3-amine;
(R)-N-(1-(4-fluorophenyl)ethyl)-3-(5-(2,2,2-trifluoroethyl)pyridin-3-yl)-1,2,4-triazin-6-amine;
(R)-N-(1-(4-fluorophenyl)ethyl)-5-(6-(2,2,2-trifluoroethyl)pyridazin-4-yl)pyrazin-2-amine;
2,2,2-trifluoro-1-(5-(5-(((R)-1-(4-fluorophenyl)ethyl)amino)pyrazin-2-yl)pyridazin-3-yl)ethan-1-ol;
(R)-5-(6-((1-(4-fluorophenyl)ethyl)amino)pyridin-3-yl)benzo[d]oxazol-2(3H)-one;
(R)-5-(6-((1-(4-fluorophenyl)ethyl)amino)pyridin-3-yl)-3-methylbenzo[d]oxazol-2(3H)-one;
(R)-6-(6-((1-(4-fluorophenyl)ethyl)amino)pyridin-3-yl)benzo[d]oxazol-2(3H)-one;
(R)-5-(5-((1-(4-fluorophenyl)ethyl)amino)pyrazin-2-yl)-3-methylbenzo[d]oxazol-2(3H)-one;
(R)-5-(2-((1-(4-fluorophenyl)ethyl)amino)pyrimidin-5-yl)-3-methylbenzo[d]oxazol-2(3H)-one;
(R)-5-(3-((1-(4-fluorophenyl)ethyl)amino)-1,2,4-triazin-6-yl)-3-methylbenzo[d]oxazol-2(3H)-one;
(R)-5-(3-((1-(4-fluorophenyl)ethyl)amino)-1,2,4-triazin-6-yl)-3-(2-hydroxyethyl)benzo[d]oxazol-2(3H)-one;
(R)-5-(3-((1-(4-fluorophenyl)ethyl)amino)-1,2,4-triazin-6-yl)-3-(methoxymethyl)benzo[d]oxazol-2(3H)-one;
(R)-3-ethyl-5-(3-((1-(4-fluorophenyl)ethyl)amino)-1,2,4-triazin-6-yl)benzo[d]oxazol-2(3H)-one;
(R)-3-(difluoromethyl)-5-(3-((1-(4-fluorophenyl)ethyl)amino)-1,2,4-triazin-6-yl)benzo[d]oxazol-2(3H)-one;
(R)-5-(3-((1-(4-fluorophenyl)ethyl)amino)-1,2,4-triazin-6-yl)benzo[d]oxazol-2(3H)-one;
(R)-5-(3-((1-(4-fluorophenyl)ethyl)amino)-1,2,4-triazin-6-yl)-3-((methylthio)methyl)benzo[d]oxazol-2(3H)-one;
(R)-3-(2-(dimethylamino)ethyl)-5-(3-((1-(4-fluorophenyl)ethyl)amino)-1,2,4-triazin-6-yl)benzo[d]oxazol-2(3H)-one;
(R)-5-(3-((1-(4-fluorophenyl)ethyl)amino)-1,2,4-triazin-6-yl)-3-((methylsulfonyl)methyl)benzo[d]oxazol-2(3H)-one;
(R)-5-(3-((1-(4-chlorophenyl)ethyl)amino)-1,2,4-triazin-6-yl)-3-methylbenzo[d]oxazol-2(3H)-one;
4-(1-((6-(3-methyl-2-oxo-2,3-dihydrobenzo[d]oxazol-5-yl)-1,2,4-triazin-3-yl)amino)ethyl)benzonitrile;
(R)-5-(3-((1-(2,4-difluorophenyl)ethyl)amino)-1,2,4-triazin-6-yl)-3-methylbenzo[d]oxazol-2(3H)-one;
(R)-5-(3-((1-(3,4-difluorophenyl)ethyl)amino)-1,2,4-triazin-6-yl)-3-methylbenzo[d]oxazol-2(3H)-one;
5-(3-((1-(4-fluoro-3-methylphenyl)ethyl)amino)-1,2,4-triazin-6-yl)-3-methylbenzo[d]oxazol-2(3H)-one;
5-(3-((1-(3,4-dichlorophenyl)ethyl)amino)-1,2,4-triazin-6-yl)-3-methylbenzo[d]oxazol-2(3H)-one;
(R)-3,3-difluoro-6-(6-((1-(4-fluorophenyl)ethyl)amino)pyridin-3-yl)-1-methylindolin-2-one;
(R)-6-(benzo[d]oxazol-5-yl)-N-(1-(4-fluorophenyl)ethyl)-1,2,4-triazin-3-amine;
(R)-N-(1-(4-fluorophenyl)ethyl)-6-(1-methyl-1H-benzo[d]imidazol-6-yl)-1,2,4-triazin-3-amine;
(R)-N-(1-(4-fluorophenyl)ethyl)-6-(1-methyl-1H-imidazo[4,5-b]pyridin-6-yl)-1,2,4-triazin-3-amine;
(R)-N-(1-(4-fluorophenyl)ethyl)-6-(3-methyl-3H-imidazo[4,5-b]pyridin-6-yl)-1,2,4-triazin-3-amine;
or an enantiomer of any of the foregoing;
or a pharmaceutically acceptable salt, solvate or prodrug of any of the foregoing.

13. A process for the preparation of the compound, salt, solvate or prodrug as claimed in claim 1, wherein the process comprises:
(a) reacting a compound of formula (II) or a salt thereof, with a compound of formula (III) or a salt thereof: 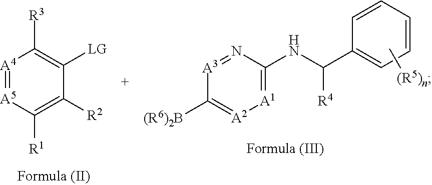
or
(b) reacting a compound of formula (IV) or a salt thereof, with an amine of formula (V) or a salt thereof: 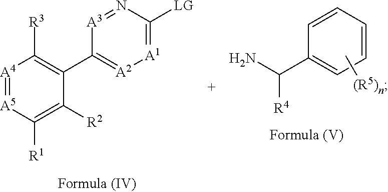
or
(c) reacting a compound of formula (VI) or a salt thereof, with a compound of formula (VII) or a salt thereof: 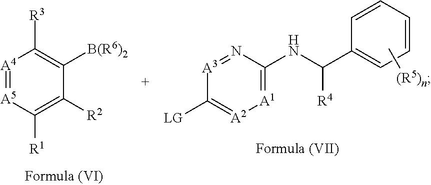
wherein:
R¹, R², R³, R⁴, R⁵, A¹, A², A³, A⁴ and A⁵ are as defined in claim 1 or can be converted into such a group;
n is as defined in claim 1;
R⁶ is independently selected from hydroxyl, C₁-C₅ alkoxy or C₁-C₅ alkyl, or two R⁶ together with the boron to which they are attached form an optionally substituted 5- to 6-membered heterocyclic group; and
LG is a leaving group;
and optionally thereafter carrying out one or more of the following procedures:
converting a compound of formula (I), (Ia) or (Ib) into another compound of formula (I), (Ia) or (Ib);
removing any protecting groups;
forming a pharmaceutically acceptable salt.

14. A pharmaceutical composition comprising the compound, salt, solvate or prodrug as claimed in claim 1, in association with a pharmaceutically acceptable adjuvant, diluent or carrier, and optionally one or more other therapeutic agents.

15. The compound, salt, solvate or prodrug as claimed in claim 1, for use in therapy.

16. A method of treating or preventing a disease, disorder or condition associated with GPR55 activity, the method comprising administering a therapeutically or prophylactically effective amount of the compound, salt, solvate or prodrug as claimed in claim 1 to a patient in need thereof.

17. A method of treating or preventing a neurodegenerative disease, Parkinson's Disease, Alzheimer's Disease, depression, anxiety, an anxiety related disorder, epilepsy, or pain including mechanical, inflammatory and neuropathic pain, the method comprising administering a therapeutically or prophylactically effective amount of the compound, salt, solvate or prodrug as claimed in claim 1 to a patient in need thereof.
